SEQUENCE LISTING (3 sequences)

SEQ ID NO: 1 - DNA, 21 bp, synthetic construct
  source 1..21: mol_type = other DNA
gagataagcattataattcct

SEQ ID NO: 2 - DNA, 20 bp, synthetic construct
  source 1..20: mol_type = other DNA
tcagggtctcacaattgggc

SEQ ID NO: 3 - DNA, 20 bp, synthetic construct
  source 1..20: mol_type = other DNA
gcaccagctcacgttgacgt

(19) United States
(12) Patent Application Publication
(10) Pub. No.: US 2026/0232677 A1
(43) Pub. Date: Aug. 13, 2026

(54) METHODS OF TREATING PROSTATE CANCER BY INHIBITING NUCLEAR RECEPTOR BINDING SET DOMAIN PROTEIN 2 (NSD2)

(71) Applicant: The Trustees of Columbia University in the City of New York, New York, NY (US)

(72) Inventors: Michael M. SHEN, New York, NY (US); Jia LI, New York, NY (US)

(21) Appl. No.: 19/074,269

(22) Filed: Mar. 7, 2025

Related U.S. Application Data
(63) Continuation-in-part of application No. PCT/US2023/073895, filed on Sep. 11, 2023.
(60) Provisional application No. 63/375,217, filed on Sep. 9, 2022.
(60) Provisional application No. 63/490,756, filed on Mar. 16, 2023.

(51) Int. Cl. A61K 31/52; A61K 31/498; A61K 31/506; A61K 31/538; A61K 31/7105; A61K 45/06; A61P 35/00; C12N 15/113

(52) U.S. Cl. CPC A61K 31/52; A61K 31/498; A61K 31/506; A61K 31/538; A61K 31/7105; A61K 45/06; A61P 35/00; C12N 15/113; C12N 2310/14

(57) ABSTRACT

The subject matter described herein relates to a method of treating or preventing prostate cancer in a subject with administration of a composition comprising a Nuclear Receptor Binding Set Domain Protein 2 (NSD2) inhibitor.

This application is a continuation-in-part of PCT/US2023/073895, filed Sep. 11, 2023, which claims the benefit of and priority to U.S. Provisional Patent Application No. 63/375,217, filed on Sep. 9, 2022, and U.S. Provisional Patent Application No. 63/490,756, filed on Mar. 16, 2023, the contents of each of which are hereby incorporated by reference in their entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### GOVERNMENT SUPPORT

This invention was made with government support under Grant No. CA251527 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created on Apr. 27, 2026, is named 0019240_01290US3_SL.xml and is 3,775 bytes in size.

### SUMMARY OF THE INVENTION

In certain aspects, the subject matter described herein provides a method of treating or preventing prostate cancer in a subject in need thereof, the method comprising administering to the subject a pharmaceutical composition comprising a therapeutically effective amount of a Nuclear Receptor Binding Set Domain Protein 2 (NSD2) inhibitor.

In some embodiments, the prostate cancer is an advanced stage or an end-stage cancer. In some embodiments, the prostate cancer is castration-resistant prostate cancer (CRPC). In some embodiments, the prostate cancer is a neuroendocrine prostate cancer (NEPC). In some embodiments, the NEPC corresponds to a treatment-related neuroendocrine castration-resistant prostate cancer (CRPC-NE). In some embodiments, the NEPC is a primary de novo neuroendocrine prostate tumor. In some embodiments, the CRPC lacks androgen receptor expression and/or sensitivity to one or more androgen receptor inhibitors. In some embodiments, the CRPC is CRPC with decreased or no AR expression. In some embodiments, the CRPC-NE lacks androgen receptor expression and/or sensitivity to one or more androgen receptor inhibitors. In some embodiments, the CRPC-NE is CRPC-NE with decreased or no AR expression. In some embodiments, the CRPC is CRPC with androgen receptor (AR) expression (CRPC-AR). In some embodiments, CRPC is CRPC with an activation of the Wnt signaling pathway (CRPC-WNT). In some embodiments the CRPC-WNT lacks androgen receptor expression and/or sensitivity to one or more androgen receptor inhibitors. In some embodiments, the CRPC-WNT is CRPC-WNT with decreased or no AR expression. In some embodiments, the CRPC is CRPC with stem cell-like characteristics (CRPC-SCL). In some embodiments the CRPC-SCL lacks androgen receptor expression and/or sensitivity to one or more androgen receptor inhibitors. In some embodiments, the CRPC-SCL is CRPC-SCL with decreased or no AR expression.

In some embodiments, the NSD2 inhibitor comprises UNC6934. In some embodiments, the inhibitor is administered in combination with another NSD2 inhibitor or one or more NSD2 activity modulator. In some embodiments, the inhibitor comprises a composition comprising a small interfering RNA specific for a messenger RNA sequence encoding the NSD2 protein. In some embodiments, the inhibitor comprises a CRISPR cassette specific for the nucleotide gene sequence encoding a NSD2 gene or controlling transcription of a NSD2 gene. In some embodiments, the NSD2 inhibitor comprises 3-hydrazinoquinoxaline-2-thiol (MCTP-39). In some embodiments, the NSD2 inhibitor comprises KTX-1001. In some embodiments, the NSD2 inhibitor comprises MS159.

In some embodiments, the pharmaceutical composition is administered before, after, or in combination with an anti-androgen treatment. In some embodiments, the anti-androgen treatment comprises administration of enzalutamide.

In some embodiments, the anti-androgen treatment comprises administration of abiraterone. In some embodiments, the anti-androgen treatment comprises administration of apalutamide. In some embodiments, the anti-androgen treatment comprises administration of bicalutamide. In some embodiments, the anti-androgen treatment comprises administration of darolutamide. In some embodiments, the anti-androgen treatment comprises administration of flutamide. In some embodiments, the anti-androgen treatment comprises administration of nilutamide. In some embodiments, the anti-androgen treatment comprises administration of one or more luteinizing hormone-releasing hormone (LHRH) agonists.

In some embodiments, the composition is administered before, after, or in combination with radiation therapy. In some embodiments, administration of the NSD2 inhibitor to the subject in combination with an anti-androgen treatment decreases tumor size. In some embodiments, administration of the NSD2 inhibitor to the subject in combination with an anti-androgen treatment decreases tumor number. In some embodiments, the administration of the NSD2 inhibitor to the subject in combination with an anti-androgen treatment prevents cancer metastasis.

In certain aspects, the subject matter described herein provides a method of inducing prostate cancer sensitivity to one or more androgen receptor (AR) inhibitors in a subject in need thereof, the method comprising administering to the subject a pharmaceutical composition comprising a therapeutically effective amount of a Nuclear Receptor Binding Set Domain Protein 2 (NSD2) inhibitor.

In some embodiments, the prostate cancer is an advanced stage or an end-stage cancer. In some embodiments, the prostate cancer is castration-resistant prostate cancer (CRPC). In some embodiments. In some embodiments, the prostate cancer is a neuroendocrine prostate cancer (NEPC). In some embodiments, the NEPC corresponds to a treatment-related neuroendocrine castration-resistant prostate cancer (CRPC-NE). In some embodiments, the NEPC is a primary de novo neuroendocrine prostate tumor. In some embodiments, the CRPC lacks androgen receptor expression and/or sensitivity to one or more androgen receptor inhibitors. In some embodiments, the CRPC is CRPC with decreased or no AR expression. In some embodiments, the CRPC-NE lacks androgen receptor expression and/or sensitivity to one or more androgen receptor inhibitors. In some embodiments, the CRPC-NE is CRPC-NE with decreased or no AR expression. In some embodiments, the CRPC is CRPC with AR expression (CRPC-AR). In some embodiments, CRPC is CRPC with an activation of the Wnt signaling pathway (CRPC-WNT). In some embodiments the CRPC-WNT has decreased or no androgen receptor expression and/or sensitivity to one or more androgen receptor inhibitors. In some embodiments, the NSD2 inhibitor comprises UNC6934. In some embodiments, the NSD2 inhibitor is administered in combination with another NSD2 inhibitor or one or more NSD2 activity modulator. In some embodiments, the inhibitor comprises a composition comprising a small interfering RNA specific for a messenger RNA sequence encoding the NSD2 protein. In some embodiments, the inhibitor comprises a CRISPR cassette specific for the nucleotide gene sequence encoding a NSD2 gene or controlling transcription of a NSD2 gene. In some embodiments, the NSD2 inhibitor comprises 3-hydrazinoquinoxaline-2-thiol (MCTP-39). In some embodiments, the NSD2 inhibitor comprises KTX-1001. In some embodiments, the NSD2 inhibitor comprises MS159. In some embodiments, the AR inhibitor comprises enzalutamide. In some embodiments, the composition is administered before, after, or in combination with radiation therapy. In some embodiments, the subject is a human.

### BRIEF DESCRIPTION OF FIGURES

The patent or application file contains at least one drawing executed in color. Copies of this patent or patent application publication with color drawing(s) will be provided by the Office upon request and payment of the necessary fee.

FIGS. 1A-D show that organoids from NPp53 mice recapitulate heterogeneity of CRPC-NE. A, Hematoxylin and eosin (H & E) and immunofluorescence staining of sections from parental tumors and matched NPPO organoid lines established from NPp53 mice at passage 2. AR, Androgen receptor; CHGA, Chromogranin A; SYP, Synaptophysin; VIM, Vimentin. B, Schematic depiction of co-culture assay for neuroendocrine transdifferentiation. C, D, Immunofluorescence analysis of RFP-expressing NPPO-1nonNE and NPPO-1NE organoids cultured separately (C) and together in organoid chimeras (D) for 4 passages. Arrows indicate cells co-expressing RFP and VIM together with the neuroendocrine markers CHGA and SYP. Scale bars in a,c,d indicate 50 microns.

FIGS. 2A-D show that NSD2 inhibition reverts neuroendocrine differentiation. A, B, D, Hematoxylin-and-eosin (H & E) staining and immunofluorescence staining of sections from NPPO-1NE (A), NPPO-2 (B), or MSKPCa10 (D) organoids following CRISPR-mediated knock-out of Nsd2 (sgNsd2) or control (sgCtrl), or NPPO-6 organoids (C) after transfection of the oncohistone H3.3K36M or control (EV, empty vector). AR, Androgen receptor; BSD, Blasticidin (drug selection marker); CHGA, Chromogranin A; CK8, Cytokeratin 8; HA, Hemagglutinin tag; SYP, Synaptophysin, VIM, Vimentin. Scale bars indicate 50 microns.

FIGS. 3A-H show that NSD2 inhibition restores enzalutamide response in neuroendocrine organoids and grafts. A, Dose-response curves for enzalutamide treatment of Nsd2 knock-out (sgNsd2) or control (sgCtrl) NPPO-1NE and NPPO-2 organoids and for control (EV, empty vector) or H3.3K36M transfected NPPO-4 and NPPO-6 organoids. IC50 values were calculated from dose-response curves by nonlinear regression (curve fit). Each data point corresponds to three biological replicates; error bars represent one standard deviation. Dose-response curves were compared by two-way ANOVA. B, Tumor growth curves for the same lines as in a, except treated with enzalutamide or DMSO control in vivo starting at day 14 after subcutaneous grafting in NOD/SCID mice at day 0. Each data point corresponds to five biological replicates; error bars represent one standard deviation. Unpaired t-tests (two-tailed P value) were used to compare the means between two groups. C, D, E, H & E and immunofluorescence analysis of sections from NPPO-1NE (C), NPPO-6 (D), and MSKPCa10 grafts (E). CHGA, Chromogranin A; HA, Hemagglutinin tag. F, Dose-response curve for enzalutamide treatment of NSD2 knock-out (sgNSD2) or control (sgCtrl) MSKPCa10 organoids. IC50 values and statistics were calculated as in panel A. G, Tumor growth curves for control and NSD2 knock-out MSKPCa10 subcutaneous xenografts treated with enzalutamide or DMSO control in vivo. Each data point corresponds to six biological replicates; error bars represent one standard deviation. Unpaired t-tests (two-tailed P value) were used to compare the means between two groups. H, Model for loss of neuroendocrine differentiation and castration-resistance after NSD2 inhibition. See text for description.

FIG. 4 shows histology of parental NPp53 tumors and corresponding NPPO organoid lines. Low- and medium-power views of hematoxylin-and-eosin (H & E) stained sections from the indicated organoid lines at passage 2 and corresponding parental tumors. Scale bars indicate 200 microns.

FIG. 5 show phenotypes of non-neuroendocrine NPPO organoid lines. Immunofluorescence staining for the indicated markers in non-neuroendocrine NPPO organoid lines. AR, Androgen receptor; CK8, Cytokeratin 8; VIM, Vimentin.

FIGS. 6A-C show flow sorting of NPPO organoids. A, Sorting strategy for isolation of NPPO-1NE and NPPO-1nonNE sublines from NPPO-1 organoids. B, Isolation of sgCtrl and sgNsd2 transfected cells from NPPO-1NE and NPPO-2 organoid lines. C, Flow cytometry analysis of H3.3K36M-expressing cells from NPPO-4 and NPPO-6 organoid lines.

FIGS. 7A-B show immunofluorescence screen for differential levels of epigenetic marks. A, Immunostaining of indicated epigenetic marks in NPPO-1 organoids. Images are shown in pairs, with and without co-staining for Vimentin (VIM). CHGA, Chromogranin A; SYP, Synaptophysin. Scale bars indicate 50 microns. B, Quantitation of epigenetic mark levels, comparing fluorescence intensity in neuroendocrine (NE) and non-neuroendocrine (non-NE) cells in three replicate experiments. Mean fluorescence intensities were compared by unpaired t-tests (two-tailed P value); comparisons lacking p-values were not significant. Unpaired t test (two tailed P value) was used for comparison between two groups.

FIG. 8 shows that NSD2 inhibition reverts neuroendocrine differentiation. Hematoxylin-and-eosin (H & E) staining and immunofluorescence staining of sections from NPPO-4 organoids after transfection of the oncohistone H3.3K36M or control (EV, empty vector). AR, Androgen receptor; CHGA, chromogranin A; HA, Hemagglutinin tag; VIM, Vimentin. Scale bars indicate 50 microns.

FIGS. 9A-I show organoid response to combined NSD2 inhibition and enzalutamide treatment. A, B, Growth of Nsd2 knock-out (sgNsd2) or control (sgCtrl) NPPO-1NE and NPPO-2 organoids (A), control (EV, empty vector) or H3.3K36M-transfected NPPO-4 and NPPO-6 organoids (B), Experimental values were normalized to DMSO controls and shown as percentage of viable cells (n=10 biological replicates). P-values were calculated using unpaired t-tests (two-tailed). C-F, Whole-mount images of organoids following Nsd2 knock-out or H3.3K36M expression treated with either DMSO control or 10 μM enzalutamide. All NPPO organoid lines express YFP (green) due to the Cre reporter in the NPp53 mouse model; the NPPO-1NE, NPPO-2 organoids additionally express RFP following sgCtrl or sgNSD2 transfection. G, H, Growth of sgNSD2 or sgCtrl MSKPCa10 organoids treated with 3.5 μM (G) or 10 μM enzalutamide (H) or DMSO control. I, Whole-mount images of MSKPCa10 organoids following NSD2 knock-out or H3.3K36M expression treated with either DMSO control or 10 μM enzalutamide. These organoids express RFP following sgCtrl or sgNSD2 transfection.

FIGS. 10A-B show graft response to combined NSD2 inhibition and enzalutamide treatment. A, Whole-mount images of grafts following subcutaneous implantation of NPPO-1NE, NPPO-2, and MSKPCa10 control or NSD2 knock-out organoids (left) or NPPO-4 and NPPO-6 control or H3.3K36M-transfected organoids (right) into NOD/SCID immunodeficient mice treated with DMSO control or enzalutamide. B, H & E and immunofluorescence analysis of sections from NPPO-2 (left) and NPPO-4 (right) grafts. CHGA, Chromogranin A; HA, Hemagglutinin tag. Scale bars in B indicate 50 microns.

FIGS. 11A-D show that a small molecule that targets NSD2 synergizes with enzalutamide treatment to inhibit growth of mouse NPPO organoids. Cell viability of mouse NPPO organoid lines treated 300 nM UNC6934 or 10 μM enzalutamide or both for 5 days. Cell viability was measured by CellTiter-Glo assays; p values were calculated by a two-tailed t test. A, shows the mouse NPPO-1NE organoid line. B, shows the mouse NPPO-2 organoid line. C, shows the mouse NPPO-4 organoid line. D, shows the mouse NPPO-6 organoid line.

FIGS. 12A-B show that a small molecule that targets NSD2 synergizes with enzalutamide treatment to inhibit growth of human MSKPCa10 (A) and MSKPCa14 (B) organoids. Cell viability of human MSKPCa10 and MSKPCa14 organoid lines treated with 300 nM UNC6934 or 10 μM enzalutamide or both for 5 days. Cell viability was measured by CellTiter-Glo assays; p values were calculated by a two-tailed t test.

FIG. 13 shows that KTX-1001 has a synergistic effect with enzalutamide in inhibiting growth of the mouse neuroendocrine prostate cancer organoid line NPPO-6.

FIG. 14 shows antibodies used herein.

FIGS. 15-26 show small molecule inhibition of NSD2 and AR suppresses growth of human CRPC organoids and grafts. FIG. 15 is the time line of drug treatments for inhibiting NSD2 and AR in organoids and xenografts. FIGS. 16-17, western blot analyses of histone marks in the indicated human CRPC organoids after treatment with 10 μM NSD2i, a catalytic NSD2 inhibitor that is similar to the active component of KTX-1001, for 21 days (FIG. 16) or in MSKPCa10 xenografts grown for 21 days followed by treatment with the indicated doses of NSD2iA for 5 days (FIG. 17). FIG. 18, Human CRPC organoid lines utilized in this study, showing CRPC subtypes and relevant mutational features, adapted from Tang et al. (2022). FIGS. 19-20, Hematoxylin-eosin (H & E) and immunofluorescence staining of sections from WCM1262 (FIG. 19) and MSKPCa2 (FIG. 20) organoids after 21 days treatment with 3 μM (FIG. 19) and 1 μM (FIG. 20) NSD2iA. AR, Androgen receptor; CC3, cleaved caspase 3; CK8/18, cytokeratin 8 or 18. Scale bars, 50 μm. FIGS. 21A-D, Dose-response curve for cell viability following NSD2iA and enzalutamide treatment of the indicated human CRPC organoid lines. Organoids were pre-treated with the indicated concentrations of NSD2iA for 21 days prior to combined enzalutamide and NSD2iA treatment for 5 days, followed by analysis. FIGS. 22A-D, Synergy analysis of the dose-response data in FIGS. 21A-D, with calculated Bliss synergy scores and 95% confidence intervals (CI) shown. FIGS. 23A-D, Dose-response curves for cellular apoptosis following NSD2iA and enzalutamide treatment of organoid lines. Organoids were pre-treated with the indicated concentrations of NSD2iA for 21 days prior to combined enzalutamide and NSD2iA treatment for 24-48 h, followed by analysis. FIGS. 24A-D, Tumor growth curves for the indicated organoid lines grafted subcutaneously into immunodeficient NOD/SCID mice. Daily treatment with NSD2iA (150 mg/kg) or vehicle control was initiated at day 14 and continued for 2 weeks, followed by splitting into four cohorts corresponding to: 1) control; 2) daily treatment with NSD2iA alone; 3) daily treatment with enzalutamide (10 mg/kg) alone; and 4) daily treatment with NSD2iA and enzalutamide. Treatment was continued for an additional 4 weeks, with analysis at day 56. Each data point corresponds to six biological replicates; error bars represent one standard deviation. Two-way ANOVA and Tukey's multiple comparison post-hoc testing were used to compare differences between means. FIGS. 25-26, H & E and immunofluorescence staining of sections from xenografts of MSKPCa10 (FIG. 25) and MSKPCa14 (FIG. 26). CC3, cleaved caspase 3; CHGA, Chromogranin A; SYP, Synaptophysin. Scale bars, 50 μm.

FIGS. 27-34 show the activities of a small molecule inhibitor of NSD2. FIGS. 27-28, NSD2iA IC50 values for the indicated lysine methyltransferases (KMTs). Data are presented as mean±SEM, n=3 independent replicates. FIGS. 29A-D, Dose-response curves for cell viability following NSD2iA and enzalutamide treatment of NPPO-1NE, NPPO-2, NPPO-4, and NPPO-6 mouse CRPC-NE organoids. Organoids were pre-treated with 1 μM NSD2iA for 12 days prior to enzalutamide and NSD2iA treatment for 5 days. IC50 values were calculated by nonlinear regression (curve fit). Each data point corresponds to three biological replicates; error bars represent one standard deviation. Dose-response curves were compared by two-way ANOVA. FIGS. 30A-B, Dose-response curve for cell viability following NSD2iA and enzalutamide treatment of the human CRPC-NE organoid line WCM154. Organoids were pre-treated with the indicated concentrations of NSD2iA for 21 days prior to enzalutamide and NSD2iA treatment for 5 days, followed by analysis. Synergy analysis is shown below, with calculated Bliss synergy score and 95% confidence intervals (CI) shown. FIGS. 31A-B, Dose-response curve for cell viability following combined treatment of the human CRPC-NE organoid line MSKPCa10 with the EZH2 inhibitor PF-06821497 and enzalutamide. Organoids were pre-treated with the indicated concentrations of PF-06821497 for 21 days prior to enzalutamide and PF-06821497 treatment for 5 days. No synergy was detected. FIG. 32, Dose-response curve for cellular apoptosis following NSD2iA and enzalutamide treatment of WCM154. Organoids were pre-treated with the indicated concentrations of NSD2iA for 21 days prior to enzalutamide and NSD2iA treatment for 24-48 h, followed by analysis. FIGS. 33A-E, Quantitation of cell viability in the four treatment conditions utilized in the indicated organoid lines, combining all enzalutamide concentrations. One-way ANOVA and Tukey's multiple comparison post-hoc testing was used to compare differences between means. FIGS. 34A-E, Quantitation of apoptosis in the four treatment conditions as in FIGS. 33A-E.

FIGS. 35-39 show the effects of the NSD2iA on in vivo xenografts of CRPC organoids. FIGS. 35A-D, Overall body weights of mice utilized in xenograft experiments. No significant differences were observed between treatment groups. FIGS. 36, Representative whole-mount images of grafts following subcutaneous implantation of the indicated organoid lines and growth for 56 days. Grafts were treated with 150 mg/kg NSD2iA and 10 mg/kg enzalutamide following the time line shown in FIG. 5a. Images are not shown for the MSKPCa2 grafts following NSD2iA treatment as they were too small to be recovered. FIGS. 37, Hematoxylin-eosin (H & E) stained sections of grafts for MSKPCa10, MSKPCa14, and WCM1262 following the indicated treatments. Note the extensive necrosis and/or fibrosis observed following combined NSD2iA and enzalutamide treatment. Scale bars, 50 μm. FIGS. 38, H & E and immunofluorescence staining of sections from WCM1262 grafts following the indicated treatments. CC3, cleaved caspase 3. Scale bars, 50 μm. FIGS. 39A-F, Quantitation of cell proliferation in MSKPCa10 and WCM1262 grafts as well as apoptosis in MSKPCa14 grafts. Each dot represents one xenograft (n=6 biological replicates). One-way ANOVA and Tukey's multiple comparison post-hoc testing was used to compare differences between means.

FIGS. 40-41 show the synthesis of a small molecule inhibitor of NSD2. FIG. 40, Chemical structure of NSD2iA. FIG. 41, Strategy for synthesis of NSD2iA, adapted with slight modifications from Deng et al. (2023). In brief, commercially available Boc protected chiral amino ketone (1) was reduced by sodium borohydride in methanol to provide the diastereomeric mixture of alcohols (2). The Boc group in 2 was then removed under mild PTSA conditions at 60° C. to provide, after basic work up, piperidine 3 as a free base. Compound 3 was then condensed with fluoropyridine (4) in N-methyl pyrrolidinone at 100° C. for 12 hours to give ester 5 in more than 65% yield for the 3 steps. Ester 5 was in turn reduced to the corresponding alcohol (6), before transformation to the respective chloride (7), which set the stage for reaction with N,N-Bis (Boc) adenine under basic conditions to get to advanced intermediate (8) in 40% yield over 3 steps. Removal of the Boc groups using 50% TFA in DCM (v:v), and hydrogenolysis of the benzyl group in (9) provided compound 10 in 30% yield over 2 steps. Preparative chiral resolution of amino alcohol (10) provided the desired R,R-compound in 28% yield.

### BACKGROUND

Prostate cancer is one of the most common types of cancer in men. Most prostate cancers grow slowly and are initially confined to the prostate gland. While confined to the prostate, prostate cancer often does not cause serious harm. There are types of prostate cancers that are aggressive and can quickly spread to other organs.

### DETAILED DESCRIPTION OF THE INVENTION

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As would be apparent to one of ordinary skill in the art, any method or composition described herein can be implemented with respect to any other method or composition described herein.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

An “effective amount”, “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration of an affliction or condition, or one or more symptoms thereof, prevent the advancement of conditions related to an affliction or condition, prevent the recurrence, development, or onset of one or more symptoms associated with an affliction or condition, or enhance or otherwise improve the prophylactic or therapeutic effect(s) of another therapy. An effective amount also includes the amount of the compound that avoids or substantially attenuates undesirable side effects.

The terms “animal,” “subject” and “patient” as used herein includes all members of the animal kingdom including, but not limited to, mammals, animals (e.g., cats, dogs, horses, swine, etc.) and humans.

### Methods of Treatment

In some embodiments, the subject matter described herein relates to prevention and/or treatment of neuroendocrine prostate cancer (NEPC). In some embodiments, the NEPC corresponds to a treatment-related neuroendocrine castration-resistant prostate cancer (CRPC-NE). In some embodiments, the NEPC is a primary de novo neuroendocrine prostate tumor. In some embodiments, the subject matter described herein relates to prevention and/or treatment of castration-resistant prostate cancer (CRPC), which can affect approximately 15,000 new patients/year in the U.S. The current standard of care for CRPC is treatment with next-generation androgen receptor inhibitors such as enzalutamide, which nearly always leads to the emergence of treatment resistant disease. Thus, there is a major unmet need for new therapeutic approaches for CRPC. In some embodiments, the subject matter described herein relates to prevention and/or treatment of neuroendocrine subtype of castration-resistant prostate cancer (CRPC-NE). In some embodiments, the CRPC-NE has missense mutation of TP53 and/or has loss of RB pathway at RNA and protein levels. In some embodiments, the CRPC-NE has deep deletion of RB1 and/or has loss of RB pathway at RNA and protein levels. In some embodiments, the CRPC-NE has a missense mutation of TP53 or deep deletion of TP53. In some embodiments, the subject matter described herein relates to prevention and/or treatment of CRPC with decreased or no AR expression. In some embodiments, the subject matter described herein relates to prevention and/or treatment of different subtypes of CRPC with decreased or no AR expression. In some embodiments, the subject matter described herein relates to prevention and/or treatment of CRPC lacking sensitivity to one or more androgen receptor inhibitors. In some embodiments, the subject matter described herein relates to prevention and/or treatment of different subtypes of CRPC lacking sensitivity to one or more androgen receptor inhibitors. In some embodiments, the subject matter described herein relates to prevention and/or treatment of CRPC that expresses elevated levels of NSD2. In some embodiments, the subject matter described herein relates to prevention and/or treatment of CRPC that is at high risk of progressing to CRPC-NE. In some embodiments, the subject matter described herein relates to prevention and/or treatment of CRPC-NE with decreased or no AR expression. In some embodiments, the subject matter described herein relates to prevention and/or treatment of CRPC-NE lacking sensitivity to one or more androgen receptor inhibitors. In some embodiments, the subject matter described herein relates to prevention and/or treatment of CRPC-NE that expresses elevated levels of NSD2. In some embodiments, the subject matter described herein relates to prevention and/or treatment of CRPC with an activation of the Wnt signaling pathway (CRPC-WNT). In some embodiments, the CRPC-WNT has deep deletion of PTEN. In some embodiments, the subject matter described herein relates to prevention and/or treatment of CRPC-WNT with decreased or no androgen receptor expression. In some embodiments, the subject matter described herein relates to prevention and/or treatment of CRPC-WNT lacking sensitivity to one or more androgen receptor inhibitors. In some embodiments, the subject matter described herein relates to prevention and/or treatment of CRPC-WNT that expresses elevated levels of NSD2. In some embodiments, the subject matter described herein relates to prevention and/or treatment of CRPC with stem cell-like characteristics (CRPC-SCL). In some embodiments the CRPC-SCL lacks androgen receptor expression and/or sensitivity to one or more androgen receptor inhibitors. In some embodiments, the CRPC-SCL is CRPC-SCL with decreased or no AR expression. In some embodiments, the subject matter described herein relates to prevention and/or treatment of CRPC with AR expression (CRPC-AR). In some embodiments, the subject matter described herein relates to prevention and/or treatment of CRPC with upregulation of downstream AR target genes. In some embodiments, the CRPC-AR has deep deletion of PTEN, missense mutation of TP53, or amplification of AR status. In some embodiments, the subject matter described herein relates to prevention and/or treatment of CRPC-AR lacking sensitivity to one or more androgen receptor inhibitors. In some embodiments, the subject matter described herein relates to prevention and/or treatment of CRPC-AR that expresses elevated levels of NSD2.

Lineage plasticity has emerged as a central mechanism that drives cancer progression and resistance to therapy, and is now considered a hallmark of cancer (see Ref 8 of Example 1). Tumor plasticity represents a formidable challenge for managing cancer care since it contributes to intra-tumor heterogeneity, promotes metastatic dissemination, and enables evasion of targeted therapy. In principle, tumor plasticity can be driven by a range of molecular mechanisms in response to cell-intrinsic changes, such as the acquisition of new mutations or epigenetic modifications, or extrinsic changes including alterations in the microenvironment or treatment regimens. Epigenetic mechanisms that drive lineage plasticity are particularly interesting since they may be more readily reversible and amenable to therapeutic interventions.

In advanced prostate cancer, progression to neuroendocrine castration-resistant prostate cancer (CRPC-NE) occurs through a lineage switch associated with transdifferentiation from luminal adenocarcinoma to neuroendocrine states (see Refs 2, 7, 9 of Example 1). Although potent androgen receptor (AR) inhibitors such as abiraterone and enzalutamide have been highly effective for treatment of hormone-sensitive prostate cancer (HSPC), most tumors inevitably develop resistance. Such castration-resistant prostate cancers (CRPC) often retain AR expression and adenocarcinoma histology (CRPC-adeno) (see Refs 1, 10 of Example 1). However, metastatic CRPC (mCRPC) can display a wide range of other tumor phenotypes due to lineage plasticity. Notably, CRPC-NE typically lacks AR expression and instead expresses neuroendocrine (NE) markers such as synaptophysin and chromogranin A4, (see Refs 5, 11 of Example 1); occasionally, it can also occur in an amphicrine form that expresses both AR and NE markers (see Ref 12 of Example 1). Yet another subtype that has been distinguished in mCRPC is double-negative prostate cancer (DNPC), which lacks both AR and NE marker expression (see Refs 13, 14 of Example 1). Importantly, the classical form of neuroendocrine prostate cancer that arises de novo in primary tumors (primary NEPC) in the absence of androgen-deprivation is relatively rare (less than 0.1% 15), whereas CRPC-NE occurs in at least 10-25% of mCRPC (see Refs 2, 3, 16-18 of Example 1).

There is considerable evidence that the lineage plasticity in CRPC-NE is mediated by epigenetic reprogramming (see Refs 2, 3, 5, 11, 19-21 of Example 1). CRPC-NE is driven by loss-of-function of the tumor suppressors TP53, RB1, and PTEN, which facilitate epigenetic reprogramming and lineage plasticity (see Refs 2, 4, 22, 23 of Example 1). In particular, mCRPC expresses high levels of EZH224, the enzymatic subunit of the Polycomb Repressive Complex 2 (PRC2), which mediates tri-methylation of histone H3 lysine 27 (H3K27me3) at the enhancers and promoters of downstream target genes. Several studies have reported that EZH2 promotes neuroendocrine differentiation in CRPC through repression of AR and luminal adenocarcinoma differentiation programs (see Refs 25-27 of Example 1).

Despite the significance of CRPC-NE, there has been a lack of useful model systems that accurately recapitulate the cell state transitions in neuroendocrine transdifferentiation and enable molecular analyses of the causal mechanisms. In some embodiments, the subject matter described herein relates to the development of new mouse organoid models to demonstrate that the histone methyltransferase NSD2 is required for maintenance of neuroendocrine differentiation as well as castration-resistance in CRPC-NE. NSD2 catalyzes the formation of H3K36me2 (see Refs 28-30 of Example 1), a histone post-translational modification associated with active chromatin. H3K36me2 has been reported to antagonize the activity of PRC2 (see Refs 31-34 of Example 1), and recruit the de novo DNA methyltransferase DNMT3A (see Refs 35-37 of Example 1), but its role in transcriptional regulation remains only partially understood. NSD2 is also known as MMSET, WHSC1, TRX5, WHS, KMT3F, KMT3G, RAUST, and REIIBP.

In some embodiments, the subject matter described herein relates to the discovery that lineage plasticity in CRPC-NE is modulated by H3K36me2 marks generated by NSD2. Genetic knock-out or oncohistone-mediated inhibition of NSD2 can revert neuroendocrine differentiation in CRPC-NE organoids and grafts, and remarkably can re-establish response to the AR inhibitor enzalutamide.

The clinical use of potent androgen receptor (AR) inhibitors has promoted the emergence of novel subtypes of metastatic castration-resistant prostate cancer (mCRPC), including neuroendocrine prostate cancer (CRPC-NE), which is highly aggressive and lethal (see Ref 1 of Example 1). These mCRPC subtypes display increased lineage plasticity and often lack AR expression (see Refs 2-5 of Example 1). In some embodiments, the subject matter described herein relates to neuroendocrine differentiation and castration-resistance in CRPC-NE. In some embodiments, neuroendocrine differentiation and castration-resistance in CRPC-NE are maintained by the activity of Nuclear Receptor Binding SET Domain Protein 2 (NSD2) (see Ref 6 of Example 1). In some embodiments, NSD2 catalyzes histone H3 lysine 36 dimethylation (H3K36me2). In some embodiments, the subject matter described herein relates to organoid lines. In some embodiments, the organoid lines are established from genetically-engineered mice (see Ref 7 of Example 1). In some embodiments, the organoid lines recapitulate key features of human CRPC-NE. In some embodiments, the organoid lines display transdifferentiation to neuroendocrine states in culture. In some embodiments, the CRPC-NE organoids express elevated levels of NSD2. In some embodiments, the CRPC-NE organoids express elevated levels of H3K36me2 marks. In some embodiments, the CRPC-NE organoids express relatively low levels of H3K27me3, consistent with antagonism of EZH2 activity by H3K36me2. In some embodiments, human CRPC-NE but not primary NEPC tumors expresses high levels of NSD2, consistent with a key role for NSD2 in lineage plasticity, and high NSD2 expression in mCRPC correlates with poor survival outcomes. In some embodiments, CRISPR/Cas9 targeting of NSD2 or expression of a dominant-negative oncohistone H3.3K36M mutant results in loss of neuroendocrine phenotypes and restores responsiveness to the AR inhibitor enzalutamide. In some embodiments, NSD2 inhibition reverses lineage plasticity and castration-resistance.

In some embodiments, the subject matter disclosed herein relates to cancer treatment. In some embodiments, the subject matter disclosed herein relates to cancer prevention. In some embodiments, the subject matter disclosed herein relates to treatment of prostate cancer. In some embodiments, the subject matter disclosed herein relates to treatment of neuroendocrine prostate cancer (NEPC). In some embodiments, the NEPC corresponds to a treatment-related neuroendocrine castration-resistant prostate cancer (CRPC-NE). In some embodiments, the NEPC is a primary de novo NEPC. In some embodiments, NEPC is a lethal end-point of advanced prostate cancer. In some embodiments, the subject matter disclosed herein relates to methods of prostate cancer prevention. In some embodiments, the subject matter disclosed herein relates to methods of NEPC prevention. In some embodiments, the NEPC corresponds to a treatment-related neuroendocrine castration-resistant prostate cancer (CRPC-NE). In some embodiments, the NEPC is a primary de novo NEPC. In some embodiments, the treatment comprises reversal of epigenetic reprogramming. In some embodiments, the epigenetic reprogramming is responsible for prostate cancer plasticity. In some embodiments, the prostate cancer plasticity occurs following anti-androgen treatment.

In certain aspects, described herein is a method of treating or preventing prostate cancer in a subject in need thereof, the method comprising administering to the subject a pharmaceutical composition comprising a therapeutically effective amount of a Nuclear Receptor Binding Set Domain Protein 2 (NSD2) inhibitor. In some embodiments, the cancer is an advanced stage or an end-stage cancer. In some embodiments, the cancer is castration-resistant prostate cancer (CRPC). In some embodiments, the CRPC-NE has missense mutation of TP53 and/or has loss of RB pathway at RNA and protein levels. In some embodiments, the CRPC-NE has deep deletion of RB1 and/or has loss of RB pathway at RNA and protein levels. In some embodiments, the CRPC-NE has missense mutation or deep deletion of TP53. In some embodiments, the prostate cancer is a neuroendocrine prostate cancer (NEPC). In some embodiments, the NEPC corresponds to a treatment-related neuroendocrine castration-resistant prostate cancer (CRPC-NE). In some embodiments, the NEPC is a primary de novo NEPC. In some embodiments, the prostate cancer is neuroendocrine CRPC (CRPC-NE). In some embodiments, the prostate cancer is CRPC with decreased or no androgen receptor expression. In some embodiments, the prostate cancer is a subtype of CRPC lacking androgen expression. In some embodiments, the prostate cancer is CRPC lacking sensitivity to one or more androgen receptor inhibitors (e.g., but not limited to, enzalutamide, abiraterone, apalutamide, bicalutamide, darolutamide, flutamide, nilutamide, luteinizing hormone-releasing hormone (LHRH) agonists). In some embodiments, the prostate cancer is a subtype of CRPC lacking sensitivity to one or more androgen receptor inhibitors (e.g., but not limited to, enzalutamide, abiraterone, apalutamide, bicalutamide, darolutamide, flutamide, nilutamide, luteinizing hormone-releasing hormone (LHRH) agonists). In some embodiments, the prostate cancer is CRPC that expresses elevated levels of NSD2 compared to non-cancerous prostate cells. In some embodiments, the prostate cancer is CRPC that is at high risk of progressing to CRPC-NE. In some embodiments, the prostate cancer is CRPC-NE with decreased or no androgen receptor expression. In some embodiments, the prostate cancer is CRPC-NE lacking sensitivity to one or more androgen receptor inhibitors. In some embodiments, the prostate cancer is CRPC-NE that expresses elevated levels of NSD2. In some embodiments, the prostate cancer is CRPC with an activation of the Wnt signaling pathway (CRPC-WNT). In some embodiments, the CRPC-WNT has deep deletion of PTEN. In some embodiments, the prostate cancer is CRPC-WNT with decreased or no androgen receptor expression. In some embodiments, the prostate cancer is CRPC-WNT lacking sensitivity to one or more androgen receptor inhibitors. In some embodiments, the prostate cancer is CRPC-WNT that expresses elevated levels of NSD2. In some embodiments, the CRPC is CRPC with AR expression (CRPC-AR). In some embodiments, the subject matter described herein relates to prevention and/or treatment of CRPC with upregulation of downstream AR target genes. In some embodiments, the CRPC-AR has deep deletion of PTEN, missense mutation of TP53, or amplification of AR status. In some embodiments, the prostate cancer is CRPC-AR lacking sensitivity to one or more androgen receptor inhibitors (e.g., but not limited to, enzalutamide, abiraterone, apalutamide, bicalutamide, darolutamide, flutamide, nilutamide, luteinizing hormone-releasing hormone (LHRH) agonists). In some embodiments, the prostate cancer is CRPC-AR that expresses elevated levels of NSD2 compared to non-cancerous prostate cells. In some embodiments, the prostate cancer is CRPC-AR that is at high risk of progressing to AR-low or AR-negative forms of CRPC, including CRPC-NE. In some embodiments, the method is a method of treating. In some embodiments, the method is a method of preventing. In some embodiments, the method is a method of preventing emergence of NEPC. In some embodiments, the method is a method of treating CRPC to prevent emergence of CRPC-NE or CRPC-AR.

In certain aspects, described herein is a method of reducing proliferation of prostate cancer cells in a subject in need thereof, the method comprising administering to the subject a pharmaceutical composition comprising a therapeutically effective amount of a Nuclear Receptor Binding Set Domain Protein 2 (NSD2) inhibitor.

In certain aspects, described herein is a method of inhibiting proliferation of prostate cancer cells in a subject in need thereof, the method comprising administering to the subject a pharmaceutical composition comprising a therapeutically effective amount of a Nuclear Receptor Binding Set Domain Protein 2 (NSD2) inhibitor.

In some embodiments, the NSD2 inhibitor comprises UNC6934. In some embodiments, the NSD2 inhibitor comprises 3-hydrazinoquinoxaline-2-thiol (MCTP-39). In some embodiments, the NSD2 inhibitor comprises KTX-1001. In some embodiments, the NSD2 inhibitor comprises MS159. In some embodiments, the composition comprises any NSD2 inhibitor known in the art or developed in the future. In some embodiments, the pharmaceutical composition further comprises one or more suitable excipients. In some embodiments, the one or more suitable excipients are known in the art.

In some embodiments, the inhibitor is administered in combination with another NSD2 inhibitor. In some embodiments, the inhibitor is administered in combination with another one or more NSD2 activity modulators.

In some embodiments, the inhibitor comprises a composition comprising a small interfering RNA specific for a messenger RNA sequence encoding the NSD2 protein. In some embodiments, the inhibitor comprises a CRISPR cassette specific for the nucleotide gene sequence encoding a NSD2 gene or controlling transcription of a NSD2 gene.

In some embodiments, the pharmaceutical composition is administered before, after, or in combination with an anti-androgen treatment. In some embodiments, the anti-androgen treatment comprises administration of enzalutamide. In some embodiments, the anti-androgen treatment comprises administration of abiraterone. In some embodiments, the anti-androgen treatment comprises administration of apalutamide. In some embodiments, the anti-androgen treatment comprises administration of bicalutamide. In some embodiments, the anti-androgen treatment comprises administration of darolutamide. In some embodiments, the anti-androgen treatment comprises administration of flutamide. In some embodiments, the anti-androgen treatment comprises administration of nilutamide. In some embodiments, the anti-androgen treatment comprises administration of one or more luteinizing hormone-releasing hormone (LHRH) agonists. In some embodiments, administration of the NSD2 inhibitor to the subject in combination with an anti-androgen treatment decreases tumor size. In some embodiments, administration of the NSD2 inhibitor to the subject in combination with an anti-androgen treatment decreases tumor number. In some embodiments, administration of the NSD2 inhibitor to the subject in combination with an anti-androgen treatment prevents cancer metastasis.

In some embodiments, the composition is administered before, after, or in combination with radiation therapy. Some non-limiting examples of conventional radiation therapy include: external beam radiation therapy, sealed source radiation therapy, unsealed source radiation therapy, particle therapy, and radioisotope therapy.

In some embodiments the subject is a mammal. In some embodiments, the subject is a mouse, a rat, a pig, a dog, a cat, or a primate. In some embodiments, the subject is a human. In some embodiments, the subject is a human patient. In some embodiments, the subject has one or more tumors. In some embodiments, the subject has cancer. In some embodiments, the subject has prostate cancer. In some embodiments, the subject has cancer which does not respond to androgen receptor inhibition treatments. In some embodiments, the subject has castration-resistant prostate cancer (CRPC). In some embodiments, the subject has neuroendocrine subtype of castration-resistant prostate cancer (CRPC-NE). In some embodiments, the subject has CRPC with AR expression (CRPC-AR).

### Methods of Inducing Prostate Cancer Sensitivity to One or More Androgen Receptor (AR) Inhibitors

In certain aspects, described herein is a method of inducing prostate cancer sensitivity to one or more androgen receptor (AR) inhibitors in a subject in need thereof, the method comprising administering to the subject a pharmaceutical composition comprising a therapeutically effective amount of a Nuclear Receptor Binding Set Domain Protein 2 (NSD2) inhibitor. In some embodiments, the cancer is an advanced stage or an end-stage cancer. In some embodiments, the cancer is castration-resistant prostate cancer (CRPC). In some embodiments, the CRPC-NE has missense mutation of TP53 and/or has loss of RB pathway at RNA and protein levels. In some embodiments, the CRPC-NE has deep deletion of RB1 and/or has loss of RB pathway at RNA and protein levels. In some embodiments, the CRPC-NE has deep deletion of TP53. In some embodiments, the prostate cancer is a neuroendocrine prostate cancer (NEPC). In some embodiments, the NEPC corresponds to a treatment-related neuroendocrine castration-resistant prostate cancer (CRPC-NE). In some embodiments, the NEPC is a primary de novo neuroendocrine prostate tumor. In some embodiments, the prostate cancer is neuroendocrine CRPC (CRPC-NE). In some embodiments, the prostate cancer is CRPC with decreased or no androgen receptor expression. In some embodiments, the prostate cancer is a subtype of CRPC with decreased or no androgen expression. In some embodiments, the prostate cancer is CRPC lacking sensitivity to one or more androgen receptor inhibitors (e.g., but not limited to, enzalutamide, abiraterone, apalutamide, bicalutamide, darolutamide, flutamide, nilutamide, luteinizing hormone-releasing hormone (LHRH) agonists). In some embodiments, the prostate cancer is a subtype of CRPC lacking sensitivity to one or more androgen receptor inhibitors (e.g., but not limited to, enzalutamide, abiraterone, apalutamide, bicalutamide, darolutamide, flutamide, nilutamide, luteinizing hormone-releasing hormone (LHRH) agonists). In some embodiments, the prostate cancer is CRPC that is at high risk of progressing to CRPC-NE. In some embodiments, the prostate cancer is CRPC that expresses elevated levels of NSD2 compared to non-cancerous prostate cells. In some embodiments, the prostate cancer is CRPC-NE with decreased or no androgen receptor expression. In some embodiments, the prostate cancer is CRPC-NE lacking sensitivity to one or more androgen receptor inhibitors. In some embodiments, the prostate cancer is CRPC-NE that expresses elevated levels of NSD2. In some embodiments, the prostate cancer is CRPC with an activation of the Wnt signaling pathway (CRPC-WNT). In some embodiments, the CRPC-WNT has deep deletion of PTEN. In some embodiments, the prostate cancer is CRPC-WNT with decreased or no androgen receptor expression. In some embodiments, the prostate cancer is CRPC-WNT lacking sensitivity to one or more androgen receptor inhibitors. In some embodiments, the prostate cancer is CRPC-WNT that expresses elevated levels of NSD2. In some embodiments, the CRPC is CRPC with AR expression (CRPC-AR). In some embodiments, the subject matter described herein relates to prevention and/or treatment of CRPC with upregulation of downstream AR target genes. In some embodiments, the CRPC-AR has deep deletion of PTEN, missense mutation of TP53, or amplification of AR status. In some embodiments, the prostate cancer is CRPC-AR lacking sensitivity to one or more androgen receptor inhibitors (e.g., but not limited to, enzalutamide, abiraterone, apalutamide, bicalutamide, darolutamide, flutamide, nilutamide, luteinizing hormone-releasing hormone (LHRH) agonists). In some embodiments, the prostate cancer is CRPC-AR that expresses elevated levels of NSD2 compared to non-cancerous prostate cells. In some embodiments, the prostate cancer is CRPC-AR that is at high risk of progressing to AR-low or AR-negative forms of CRPC, including CRPC-NE.

In some embodiments, the NSD2 inhibitor comprises UNC6934. In some embodiments, the NSD2 inhibitor comprises 3-hydrazinoquinoxaline-2-thiol (MCTP-39). In some embodiments, the NSD2 inhibitor comprises KTX-1001. In some embodiments, the NSD2 inhibitor comprises MS159. In some embodiments, the composition comprises any NSD2 inhibitor known in the art or developed in the future. In some embodiments, the pharmaceutical composition further comprises one or more suitable excipients. In some embodiments, the one or more suitable excipients are known in the art.

In some embodiments, the inhibitor is administered in combination with another NSD2 inhibitor. In some embodiments, the inhibitor is administered in combination with another one or more NSD2 activity modulators.

In some embodiments, the inhibitor comprises a composition comprising a small interfering RNA specific for a messenger RNA sequence encoding the NSD2 protein. In some embodiments, the inhibitor comprises a CRISPR cassette specific for the nucleotide gene sequence encoding a NSD2 gene or controlling transcription of a NSD2 gene.

In some embodiments, the pharmaceutical composition is administered before, after, or in combination with an AR inhibitor. In some embodiments, the AR inhibitor comprises enzalutamide. In some embodiments, the AR inhibitor comprises abiraterone. In some embodiments, the AR inhibitor comprises apalutamide. In some embodiments, the AR inhibitor comprises bicalutamide. In some embodiments, the AR inhibitor comprises darolutamide. In some embodiments, the AR inhibitor comprises flutamide. In some embodiments, the AR inhibitor comprises nilutamide. In some embodiments, the AR inhibitor comprises one or more luteinizing hormone-releasing hormone (LHRH) agonists.

In some embodiments, the composition is administered before, after, or in combination with radiation therapy. Some non-limiting examples of conventional radiation therapy include: external beam radiation therapy, sealed source radiation therapy, unsealed source radiation therapy, particle therapy, and radioisotope therapy.

In some embodiments the subject is a mammal. In some embodiments, the subject is a mouse, a rat, a pig, a dog, a cat, or a primate. In some embodiments, the subject is a human. In some embodiments, the subject is a human patient. In some embodiments, the subject has one or more tumors. In some embodiments, the subject has cancer. In some embodiments, the subject has prostate cancer. In some embodiments, the subject has cancer which does not respond to androgen receptor inhibition treatments before the administering of the pharmaceutical composition. In some embodiments, the subject has castration-resistant prostate cancer (CRPC). In some embodiments, the subject has neuroendocrine subtype of castration-resistant prostate cancer (CRPC-NE). In some embodiments, the subject has CRPC with AR expression (CRPC-AR). In some embodiments, the subject has CRPC with an activation of the Wnt signaling pathway (CRPC-WNT).

### NSD2

NSD2 is also known as MMSET, WHSC1, TRX5, WHS, KMT3F, KMT3G, RAUST, and REIIBP. Without being bound by theory, histone lysine methylation plays a role in the development of human solid tumors due primarily to its epigenetic stability. NSD2 can change methylation states leading to epigenomic changes. Specifically, NSD2 can catalyze dimethylation of histone H3 lysine 36 (H3K36me2) in mammals. There are three main isoforms of NSD2-MMSET-I (containing 647 amino acids), MMSET-II (containing 1365 amino acids) and RE-IIBP (interleukin-5 response element II-binding protein) (containing 584 amino acids). NSD2 is often overexpressed in aggressive solid tumors, including but not limited to breast cancer, renal cancer, prostate cancer, cervical cancer, and osteosarcoma. NSD2 overexpression is associated with poor cancer prognosis and recurrence. NSD2 can promote cell proliferation, cell migration, invasion, and epithelial-mesenchymal transformation (EMT), all of which are important processes in cancer metastasis. At the sub-cellular level, NSD2 plays a role in transcription activation, repression, and DNA damage repair. Signaling pathways involving NSD2 include, but are not limited to, the Wnt pathway, NF-κB signaling, and TNF& signaling.

Without being bound by theory, an epigenetic pathway regulated by NSD2 is required for neuroendocrine differentiation in mouse and human prostate cancer models. In some embodiments, inhibition of this NSD2-regulated pathway in combination with treatment with anti-androgen signaling pathway drugs such as enzalutamide can revert neuroendocrine differentiation. Therefore, the subject matter disclosed herein addresses a major unmet clinical need through treatment of castration-resistant prostate cancer (CRPC) patients to prevent the emergence of NEPC, or by treatment of NEPC patients to reverse disease progression. (Beltran et al. (2019) Clin. Cancer Res. 25:6916-6924. PMID: 31363002). The subject matter disclosed herein also addresses treatment of castration-resistant prostate cancer (CRPC) patients to prevent the emergence of AR-low or AR-negative forms of CRPC, or by treatment of CRPC-AR patients to reverse disease progression.

### Methods of Modulating NSD2 Activity

In some embodiments, the subject matter described herein relates to reversing or preventing neuroendocrine differentiation by inhibiting NSD2. In some embodiments, the subject matter described herein relates to treatment of CRPC-AR by inhibiting NSD2. In some embodiments, NSD2 inhibition also restores or induces sensitivity to enzalutamide. In some embodiments, a commercially available small molecule UNC6934 that targets NSD2 can be combined with androgen receptor inhibition (e.g., enzalutamide treatment).

Thus, NSD2 inhibitors are useful in the methods described herein. Examples of NSD2 inhibitors includes but is not limited to UNC6934 with the structure:

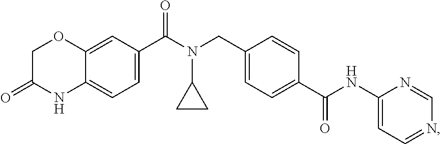 [SMILES: O=C1COc2cc(C(=O)N(Cc3ccc(C(=O)Nc4ccncn4)cc3)C3CC3)ccc2N1]

or analogs or pharmaceutically acceptable salts thereof. UNC6934 targets the N-terminal PWWP (PWWP1) domain of NSD2. UNC6934 occupies the canonical H3K36me2-binding pocket of PWWP1, antagonizes the PWWP1 interaction with nucleosomal H3K36me2 and selectively engages endogenous NSD2 in cells. UNC6934 can potently and selectively bind full-length NSD2 (fl-NSD2) in cells and induces partial disengagement from chromatin, consistent with a cooperative chromatin-binding mechanism relying on multiple protein interfaces. In some embodiments, UNC6934 lacks the ability to inhibit the catalytic histone methyltransferase activity of NSD2. In some embodiments, UNC6934 does not affect the phenotypes of t(4,14) NSD2 translocation-positive multiple myeloma cells (more information on UNC6934 can be found in Dilworth et al. (2022) Nat. Chem. Biol. 18:55-63 PMID: 34782742, the contents of which is incorporated herein in its entirety). Without being bound by theory, NSD2 may have more complex roles in advanced prostate cancer than in multiple myeloma, and therefore approaches for therapeutic targeting of NSD2 may also differ between these cancer types.

In some embodiments, the subject matter described herein relates to methods of inhibiting NSD2 activity. In some embodiments NSD2 is inhibited by small molecule inhibitors, now known or discovered in the future.

In some embodiments, the NSD2 inhibitor is a KTX-1001 compound that is an orally bioavailable inhibitor of NSD2. KTX-1001 can be referred to by its systemic name(S)-1—((R)-3-AMINO-1-(4-((6-AMINO-9H-PURIN-9-YL) METHYL)-6-(2,5-DIFLUORO-4-METHOXYPHENYL) PYRIDIN-3-YL) PIPERIDIN-3-YL)-2,2-DIFLUOROETHAN-1-OL D-TARTRATE SALT (1:1). KTX-1001 may also be referred to as EX-A5782. KTX-1001 is sometimes referred to as KTX-1001 D-tartrate. See pubchem.ncbi.nlm.nih.gov/compound/KTX-1001-D-tartrate. In some embodiments, the NSD2 inhibitor has the structure:

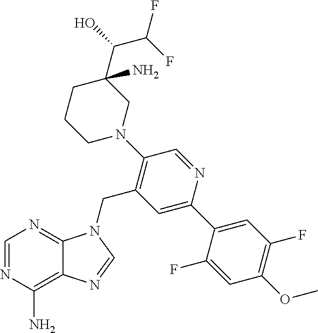 [SMILES: COc1cc(F)c(-c2cc(Cn3cnc4c(N)ncnc43)c(N3CCC[C@](N)([C@H](O)C(F)F)C3)cn2)cc1F]

or analogs or pharmaceutically acceptable salts thereof. See Deng, Haibing; Liu, Jinbiao; Oyang, Counde; Wang, Ce; Xiao, Qitao; Xun, Guoliang; Zeng, Haiqiang. Piperidinyl-methyl-purinamines as NSD2 inhibitors and anti-cancer agents and their preparation, WO2021028854 A1, and ndclist.com/unii/8t4lu8m9tg the contents of each of which are hereby incorporated by reference in their entireties.

In some embodiments, the NSD2 inhibitor is 3-hydrazinoquinoxaline-2-thiol (MCTP-39). MCT-P39 is a SAM competitor for NSD2. The SAM binding pocket is partially buried in the crystal structure of the SET domain in NSD1 and the key residues stabilizing SAM are highly conserved across the NSDs.

In some embodiments, the NSD2 inhibitor is a first-in-class NSD2 proteolysis targeting chimera (PROTAC) degrader, MS159 (more information about MS159 can be found in Meng et al. (2022) J. Med. Chem. 65:10611-10625; PMID: 35895319, which is incorporated herein in its entirety). In some embodiments. MS159 has the structure:

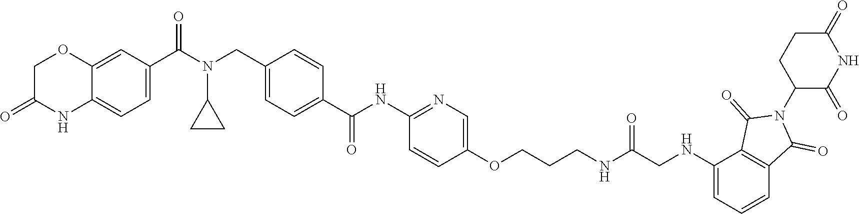 [SMILES: O=C(CNc1cccc2c1C(=O)N(C1CCC(=O)NC1=O)C2=O)NCCCOc1ccc(NC(=O)c2ccc(CN(C(=O)c3ccc4c(c3)OCC(=O)N4)C3CC3)cc2)nc1]

or analogs or pharmaceutically acceptable salts thereof.

In some embodiments, inhibitors that can be used with the subject matter described herein target the related methyltransferases NSD1 and NSD3, but have relatively little effect on NSD2. There have been several clinical trials to test the effects of Ezh2 inhibitors, which would block H3K27me3, in NEPC. Other trials are underway to examine the effects of blocking the Notch pathway in NEPC.

In some embodiments, NSD2 is inhibited through genetic alterations. In certain aspects, described herein are nucleic acids encoding RNAs of interest, which includes, but is not limited to an interfering RNA (iRNA), and variants thereof, that can silence a target gene, such as NSD2. An iRNA can down-regulate the expression of a target gene, e.g., NSD2. An iRNA may act by one or more of a number of mechanisms, including post-transcriptional cleavage of a target mRNA sometimes referred to in the art as RNAi, or pre-transcriptional or pre-translational mechanisms. An iRNA can be a double stranded (ds) iRNA. A ds iRNA includes more than one, and in certain embodiments two, strands in which interchain hybridization can form a region of duplex structure. A strand refers to a contiguous sequence of nucleotides (including non-naturally occurring or modified nucleotides). At least one strand can include a region which is sufficiently complementary to a target RNA. Such strand is termed the antisense strand. A second strand comprised in the dsRNA which comprises a region complementary to the antisense strand is termed the sense strand. However, a ds iRNA can also be formed from a single RNA molecule which is, at least partly; self-complementary, forming, e.g., a hairpin or panhandle structure, including a duplex region. In such case, the term strand refers to one of the regions of the RNA molecule that is complementary to another region of the same RNA molecule. Nonlimiting examples of inhibitory RNA include miRNA, siRNA, shRNA, and piRNA. iRNA as described herein, including ds iRNA and siRNA, can mediate silencing of a gene, e.g., by RNA degradation. In certain embodiments, the gene to be silenced is NSD2. In some embodiments, NSD2 translation is downregulated or inhibited via small interfering RNA targeting the NSD2 mRNA.

In certain embodiments, the oligonucleotide of interest is a guide RNA (gRNA) or single guide RNA (sgRNA). The CRISPR/Cas9 gene editing technique promotes a new human gene therapy strategy by correcting a defect gene at pre-chosen sites without altering the endogenous regulation of the target gene. This system consists of two key components: Cas9 protein and a guide RNA, e.g., a single guide RNA (sgRNA), as well as a correction template when needed. sgRNA contains two components: a 17-20 nucleotide sequence termed crispr RNA that is complementary to the target DNA region, and a tracr RNA that serves as the binding scaffold for a Cas nuclease. The sgRNA recognizes the target DNA and guides the Cas9 nuclease to the region for editing.

In some embodiments, NSD2 expression is downregulated or inhibited via a CRISPR/CAS9 system. Clustered regularly interspaced short palindromic repeats (CRISPR) systems are versatile gene-editing toolkits that have been used in clinical practice (more information on the clinical applications of the CRISPR/CAS9 system can be found in Stefanoudakis, D. et al. Cancers (Basel). 2023 March; 15(6): 1813, which is incorporated herein by reference).

In some embodiments, a therapeutically effective amount of a NSD2 inhibitor can be determined experimentally. In some embodiments, a therapeutically effective amount of a NSD2 inhibitor can be determined by methods known in the field. In some embodiments, the therapeutically effective amount of a NSD2 inhibitor depends on the subject of the treatment, time of administration, route of administration, duration of treatment, potency, rate of clearance and/or whether or not another drug is co-administered. These amounts can be readily determined by one of skill in the art. Any of the therapeutic applications described herein can be applied to any subject in need of such therapy, including, for example, a mammal such as a human.

In certain embodiments, a compound to be administered according to the methods described herein can be administered alone, or in combination with other drug therapies, small molecules, biologically active or inert compounds, or other additive intended to enhance the delivery, efficacy, tolerability, or function of the compound.

Pharmaceutical compositions for use in accordance with the invention can be formulated in conventional manner using one or more physiologically acceptable carriers or excipients. The therapeutic compositions of the invention can be formulated for a variety of routes of administration. Techniques and formulations generally can be found in Remington's Pharmaceutical Sciences, Meade Publishing Co., Easton, Pa (23^{rd} ed., 2020), the entire disclosure of which is herein incorporated by reference.

In some embodiments, the NSD2 inhibitors of the present disclosure can be administered through any suitable route known in the art. In some embodiments, the NSD2 inhibitors of the present disclosure can be administered orally. In some embodiments, the NSD2 inhibitors of the present disclosure can be administered parenterally. In some embodiments, the NSD2 inhibitors of the present disclosure can be administered intravenously. In some embodiments, the NSD2 inhibitors of the present disclosure can be administered nasally. In some embodiments, the NSD2 inhibitors of the present disclosure can be administered through a suppository composition.

In some embodiments, the NSD2 inhibitors of the present disclosure can be administered in any suitable administration regimen or schedule known in the art. In some embodiments, the NSD2 inhibitors of the present disclosure can be administered once daily. In some embodiments, the NSD2 inhibitors of the present disclosure can be administered twice daily. In some embodiments, the NSD2 inhibitors of the present disclosure can be administered three or more times daily. In some embodiments, the NSD2 inhibitors of the present disclosure can be administered once per week. In some embodiments, the NSD2 inhibitors of the present disclosure can be administered twice per week. In some embodiments, the NSD2 inhibitors of the present disclosure can be administered three or more times a week. In some embodiments, the NSD2 inhibitors of the present disclosure can be administered as one-time treatment procedure.

### Anti-Androgen Therapies

Anti-androgen therapy, also called androgen suppression therapy, reduces levels of the male androgen hormones in the body. The goal of this therapy is to prevent these androgens from promoting prostate cancer cell growth. The main androgens in the body are testosterone and dihydrotestosterone (DHT). Such hormone therapy is often appropriate when the cancer has spread too far throughout the body to be controlled by surgery or radiation, or if the cancer returns after treatment with surgery and/or radiation therapy. Anti-androgen therapy can also be administered before radiation to shrink the tumor size and increase treatment efficacy. Several types of anti-androgen therapy can be used to treat prostate cancer. Luteinizing hormone-releasing hormone (LHRH) agonists (also called LHRH analogs or GnRH agonists) lower the amount of testosterone produced by the testicles. Treatment with these drugs is referred to as medical castration because they lower androgen levels just as effectively as orchiectomy (surgical castration). LHRH agonists can be injected or implanted under the skin. They can be administered anywhere from once a month up to once every 6 months. The LHRH agonists include: Leuprolide (Lupron, Eligard), Goserelin (Zoladex), Triptorelin (Trelstar), Leuprolide mesylate (Camcevi). LHRH antagonists can be used to treat advanced prostate cancer. Treatment with these drugs is also a form of medical castration. Degarelix (Firmagon) can be administered as a monthly injection under the skin. Relugolix (Orgovyx) can be administered as pills, once a day. Additional therapies include Abiraterone (Zytiga), which blocks the CYP17 enzyme and stops cells from making androgens. Abiraterone can be used in men with advanced prostate cancer who are at high-risk and/or castration-resistant. Androgen receptor antagonists include, but are not limited to, Flutamide (Eulexin), Bicalutamide (Casodex), Nilutamide (Nilandron). Enzalutamide (Xtandi), apalutamide (Erleada) and darolutamide (Nubeqa) are next-generation anti-androgen therapies that can sometimes be effective where other anti-androgens have failed. For example, they can be effective in men with cancer that has not spread but is no longer responding to other forms of hormone therapy (e.g., castration-resistant prostate cancer (CRPC)). Enzalutamide can also be used for prostate cancer, whether it is castration-resistant or castration-sensitive. Apalutamide and darolutamide can also be used for castration-sensitive prostate cancer (CSPC), also known as hormone-sensitive prostate cancer (HSPC).

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1-Organoid Lines from Mouse Models of Neuroendocrine Prostate Cancer that Recapitulate Heterogeneity of Human CRPC

To study lineage plasticity in castration-resistant prostate cancer (CRPC), we have established tumor organoid lines from Nkx3.1^{CreERT2/+}; Pten^{flox/flox}, TrpP53^{flox/flox}, Rosa26-EYFP (NPp53) mice. In these mice, tamoxifen administration to adult mice results in combined deletion of the Pten and Trp53 tumor suppressor genes under the control of the Nkx3.1 promoter specifically in distal luminal epithelial cells of the prostate. In some embodiments, the subject matter described herein relates to neuroendocrine prostate cancer (NEPC), developed in NPp53 mice, that arises by trans-differentiation of luminal adenocarcinoma cells, as demonstrated by lineage-tracing of the Rosa26-EYFP reporter allele [1]. In some embodiments, the subject matter described herein relates to organoids derived from late-stage tumors that model highly aggressive advanced prostate cancer that is insensitive to anti-androgen treatments such as abiraterone or enzalutamide.

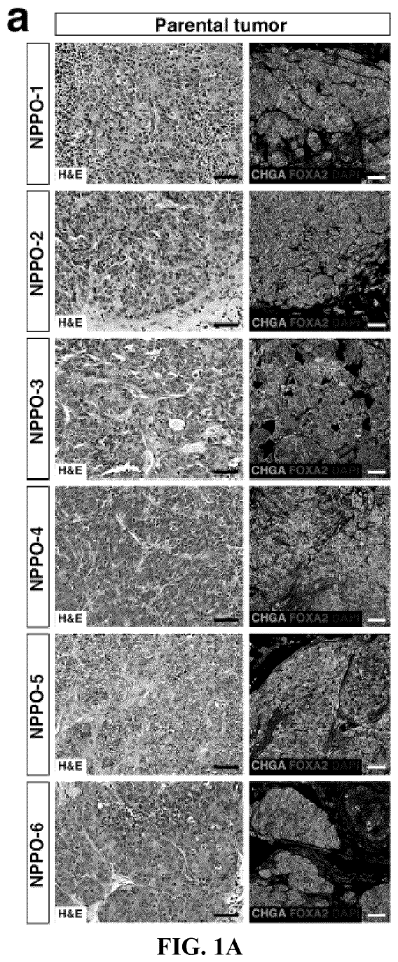

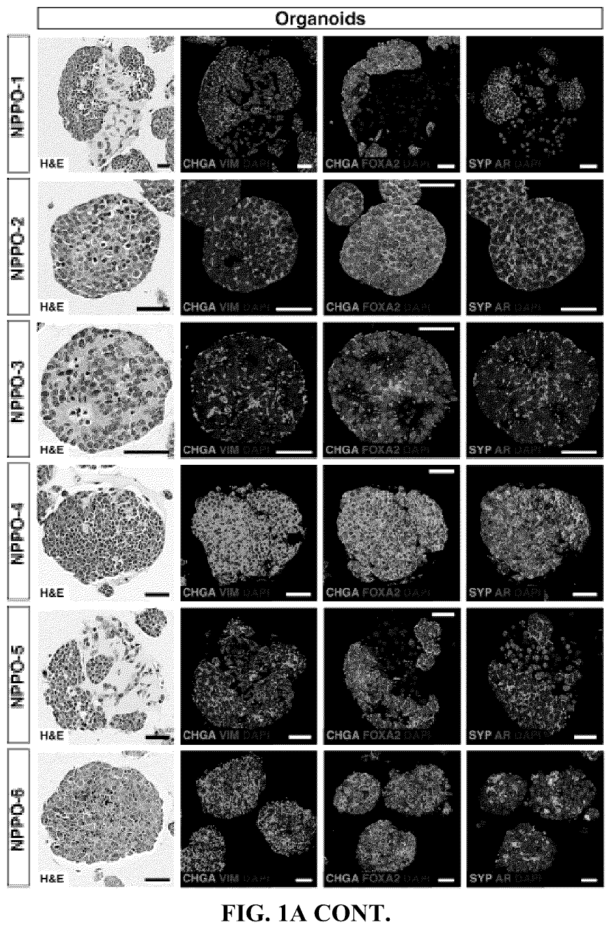

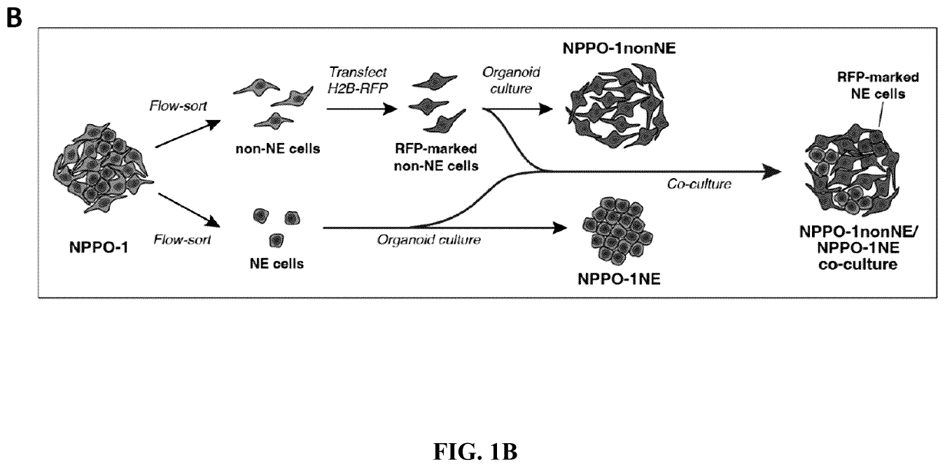

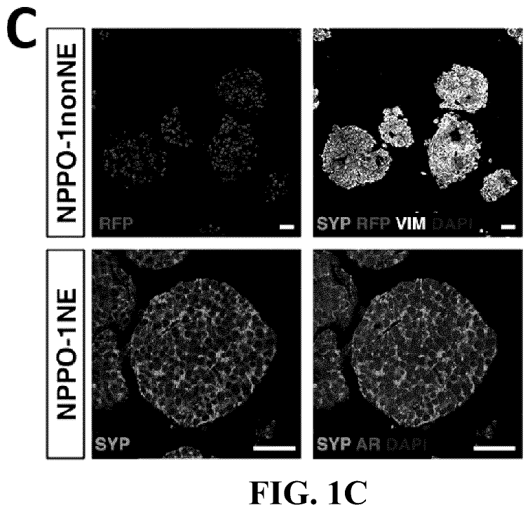

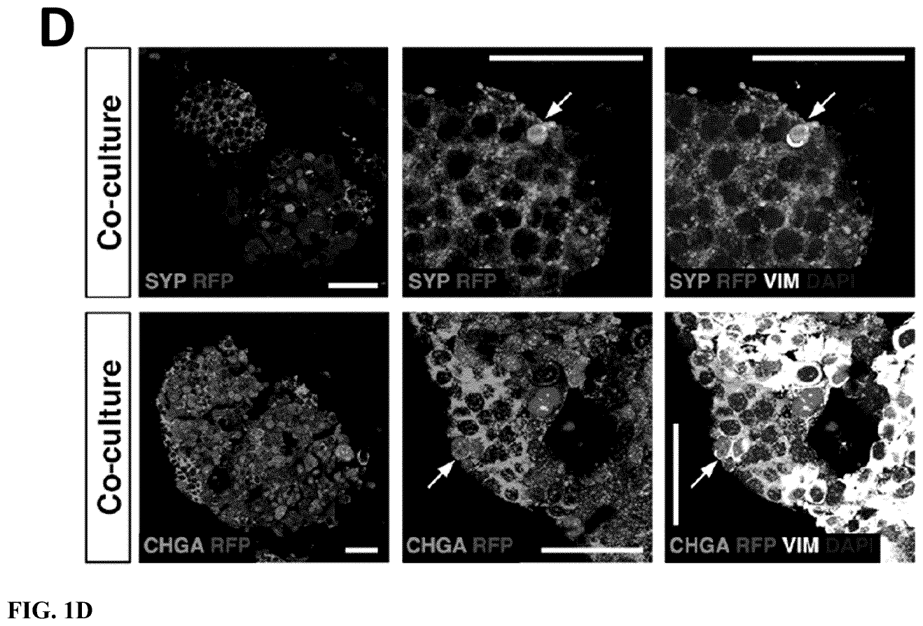

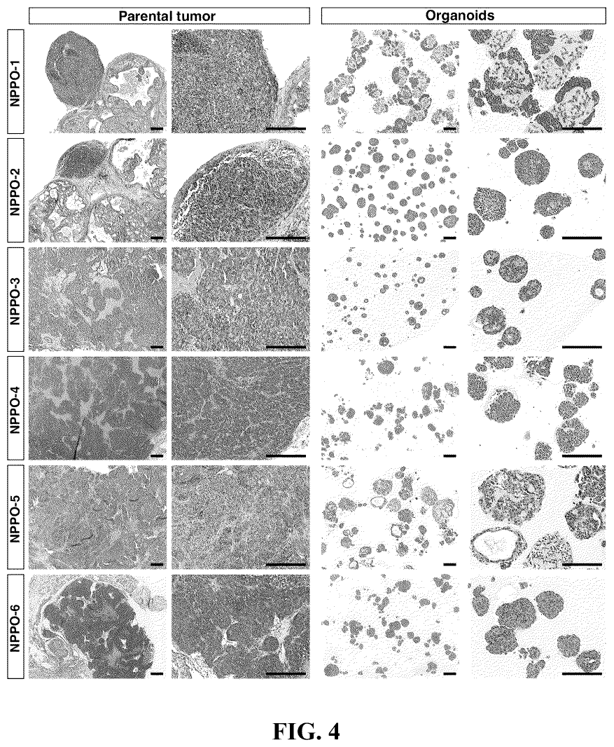

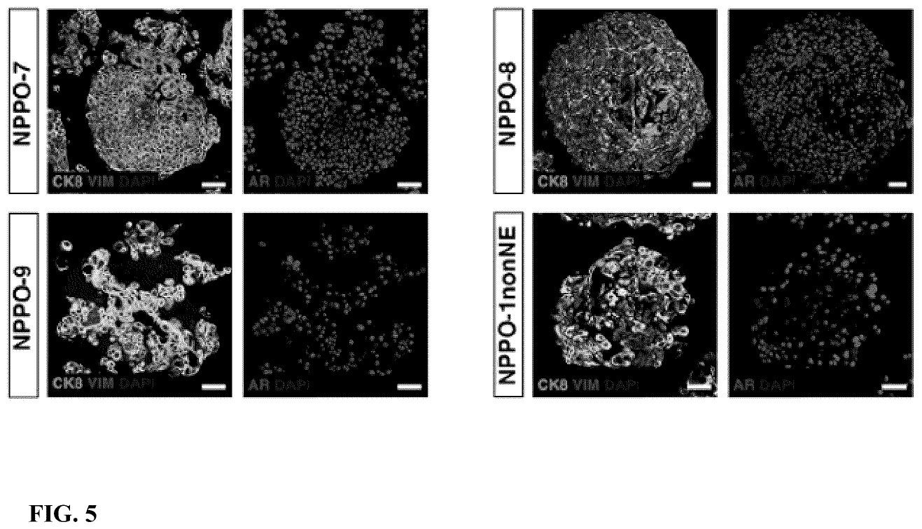

As described in Example 2, organoid lines were established from 21 independent NPp53 mice that had been induced by tamoxifen treatment at 10-12 weeks of age and harvested between 7-15 months of age using a previously described methodology [2] and analyzed their histology and marker expression by immunofluorescence staining. At these stages, the NPp53 tumors resemble highly aggressive CRPC that is insensitive to AR inhibitors such as abiraterone or enzalutamide⁷. Of these 21 lines, six lines contained cells with neuroendocrine (NE) features as determined by histopathology and marker expression and were denoted NPPO-1 (Nkx3.1, Pten, P53 Organoid line-1) through NPPO-6 (FIGS. 1A, 4). An additional 3 CRPC lines that lacked NE features were denoted NPPO-7 through NPPO-9 (FIG. 5). Five of these six lines have been passaged for greater than 21 passages and have been cryopreserved and recovered without phenotypic alterations; the NPPO-3 line could not be maintained after the first passage.

Notably, these six mouse organoid lines displayed distinctive and unique phenotypes that recapitulate many of the spectrum of human CRPC and CRPC-NE (FIG. 1A). For example, the NPPO-1 line was extremely heterogeneous, displaying a mixture of cells with a small cell histology that were positive for the neuroendocrine markers chromogranin A (Chga) and Synaptophysin (Syp) and negative for androgen receptor (AR), together with mesenchymal-like cells that were positive for AR and vimentin (Vim). The NPPO-2 line was relatively homogeneous, with most cells positive for Chga and Syp and little or no AR expression; the NPPO-3 line was similar except that Chga and Syp expression was less uniform and the organoids usually formed lumens. In contrast, the NPPO-4 line co-expressed neuroendocrine markers together with AR, corresponding to a double-positive or amphicrine state. The NPPO-5 line was also heterogeneous, but the non-neuroendocrine cells lacked mesenchymal features. Finally, the NPPO-6 line also contained amphicrine AR-positive cells that expressed Chga but displayed patchy expression of Syp. Importantly, these phenotypes could be stably maintained in culture over many passages.

### Transdifferentiation to Neuroendocrine States in Organoid Culture

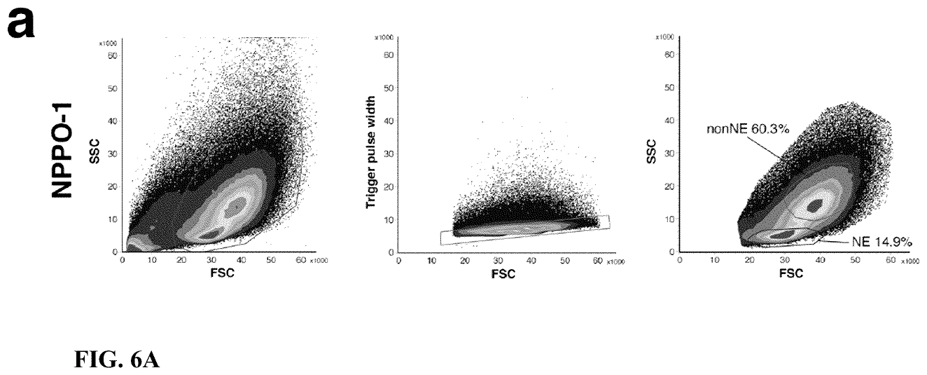

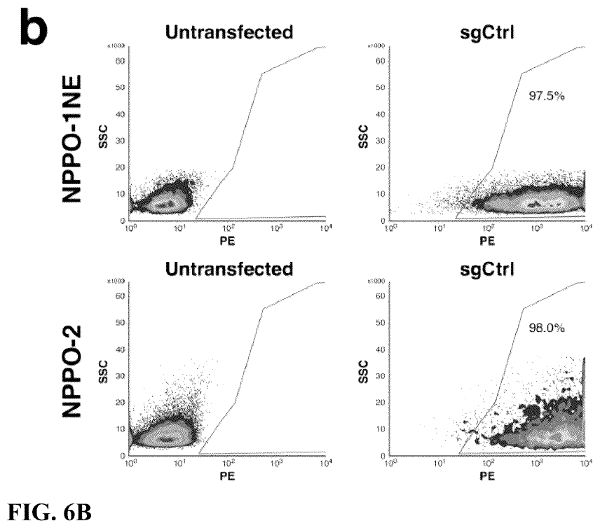

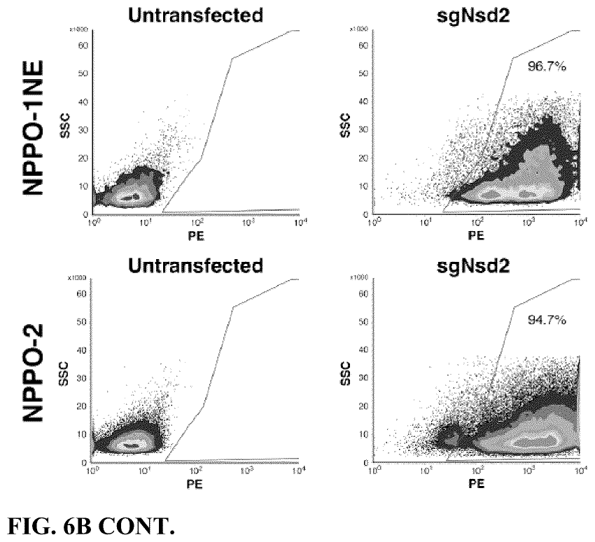

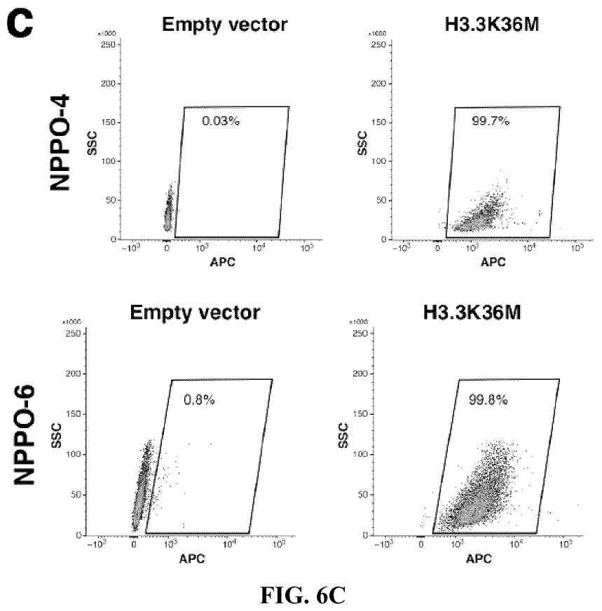

The phenotypic stability of the heterogeneous NPPO-1 organoid line suggested that its neuroendocrine (NE) and non-neuroendocrine (non-NE) populations might be maintained by paracrine interactions and/or cell state interconversions. Therefore, lineage-tracing was used to investigate whether non-NE cells could transition to NE states in culture, paralleling the transdifferentiation observed in NPp53 tumors in vivo⁷. Using flow cytometry, separate NE and non-NE populations were purified from NPPO-1 organoids, resulting in isogenic NPPO-1NE and NPPO-1nonNE sublines (FIGS. 1C and 6A; Methods). A H2BRFP expression cassette was introduced by lentiviral infection to mark the NPPO-1nonNE cells with 70% labeling efficiency (FIG. 1B). After 4 passages, NPPO-1NE and NPPO-1nonNE cells cultured separately as organoids were homogeneously neuroendocrine and non-neuroendocrine, respectively (FIG. 1C). In contrast, organoids derived from co-culture of NPPO-1NE and RFP-marked NPPO-1nonNE cells contained rare RFP-expressing cells that gained SYP or CHGA expression, indicating a shift from mesenchymal to NE states (FIG. 1D). These lineage-tracing data indicate that paracrine interactions can promote a cell state transition from an AR-positive mesenchymal state to a NE state in organoid culture.

### NSD2 and H3K36Me2 are Up-Regulated in Neuroendocrine Tumor Cells

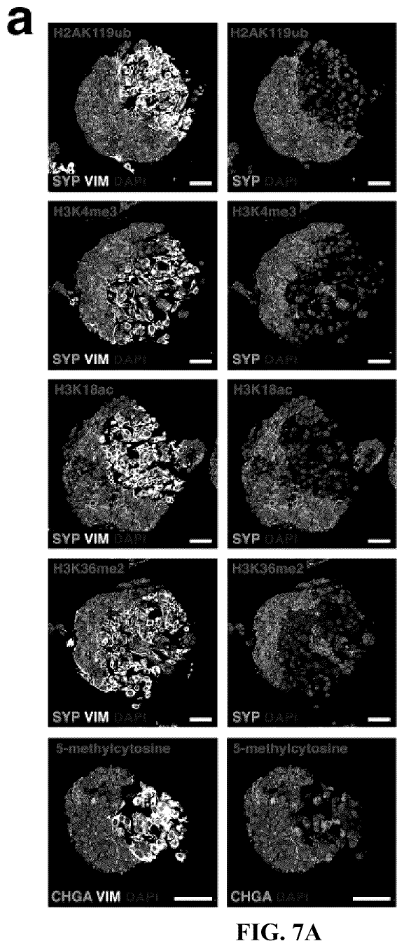

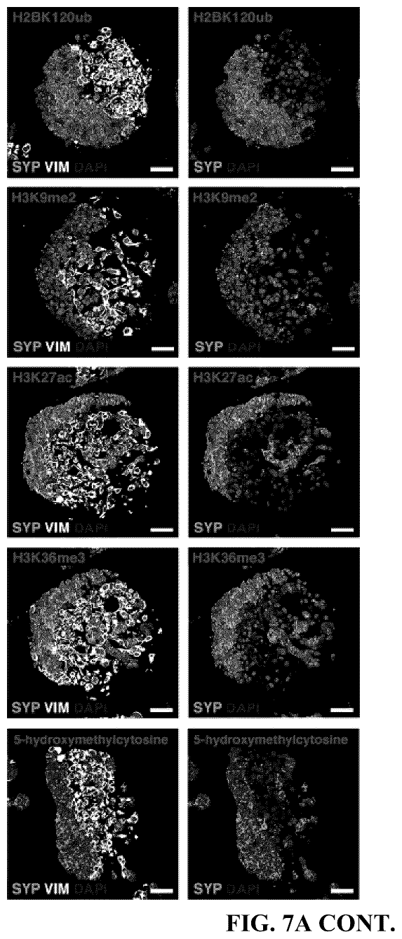

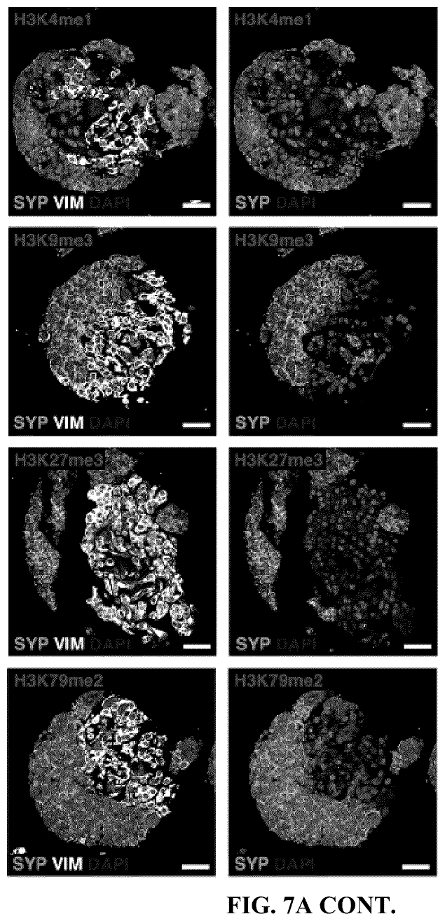

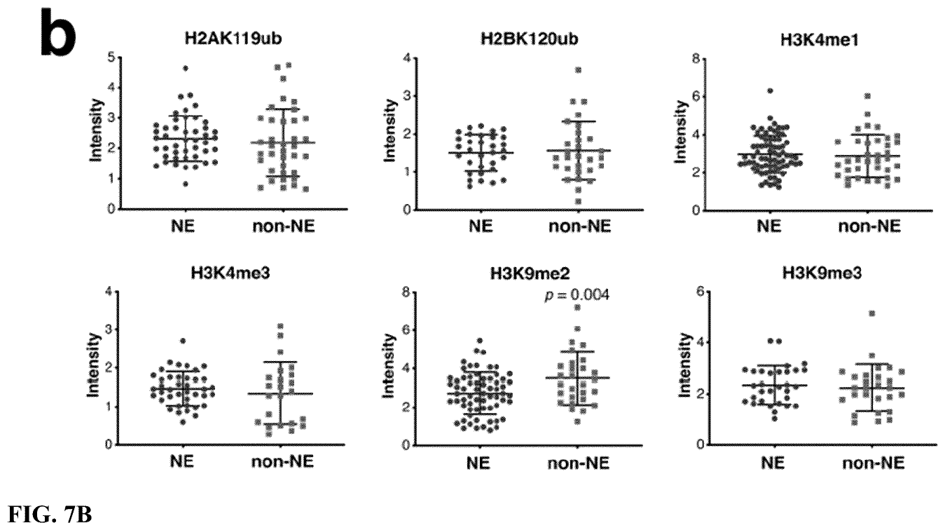

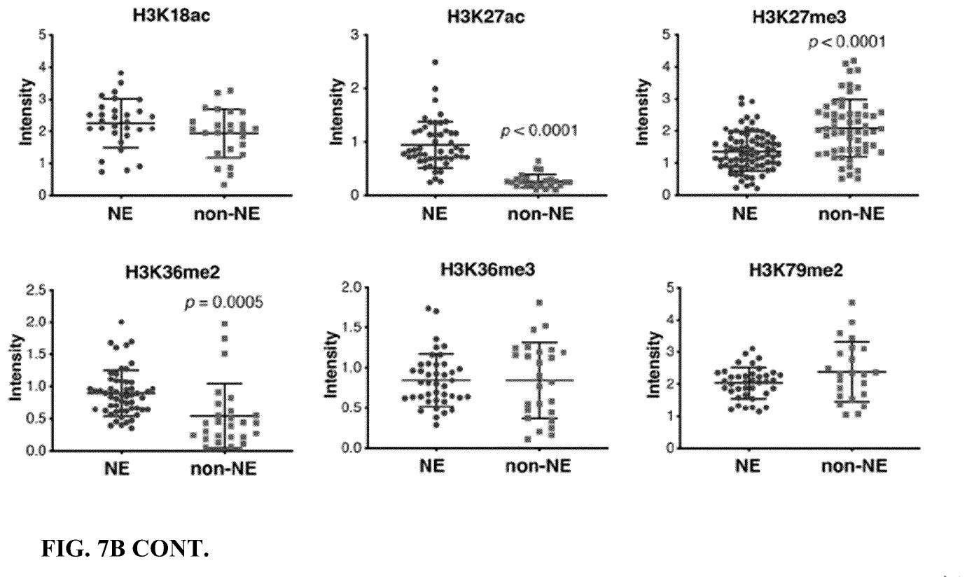

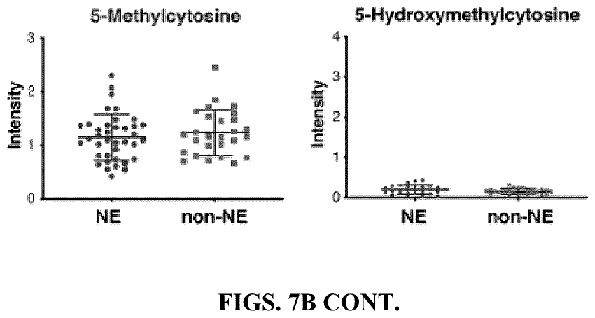

Post-translational histone modifications regulate chromatin configurations and transcriptional states. To investigate which histone modifications might be important for neuroendocrine differentiation, the levels of histone modifications were compared in NE cells versus non-NE cells of heterogeneous NPPO-1 organoids by immunofluorescence staining of histone marks and quantitated expression levels in NPPO-1 neuroendocrine cells versus non-neuroendocrine cells that expressed the mesenchymal marker vimentin (FIGS. 7A-B). Although most histone marks examined displayed similar abundance, we observed differential levels for histone H3 lysine 36 dimethylation (H3K36me2), histone H3 lysine 27 acetylation (H3K27ac), and histone H3 lysine 27 trimethylation (H3K27me3); notably, no differences were found in the levels of H3K36me3 (FIGS. 7A-B).

### NSD2 Inhibition in CRPC-NE Reverts Neuroendocrine Phenotypes

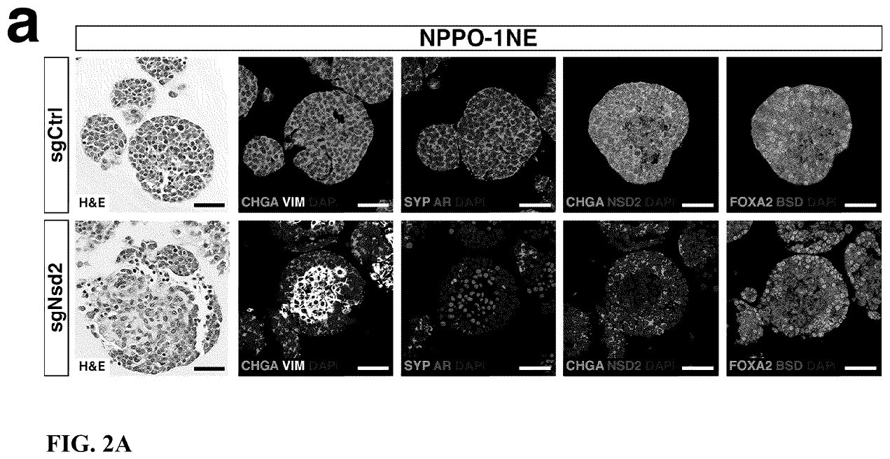

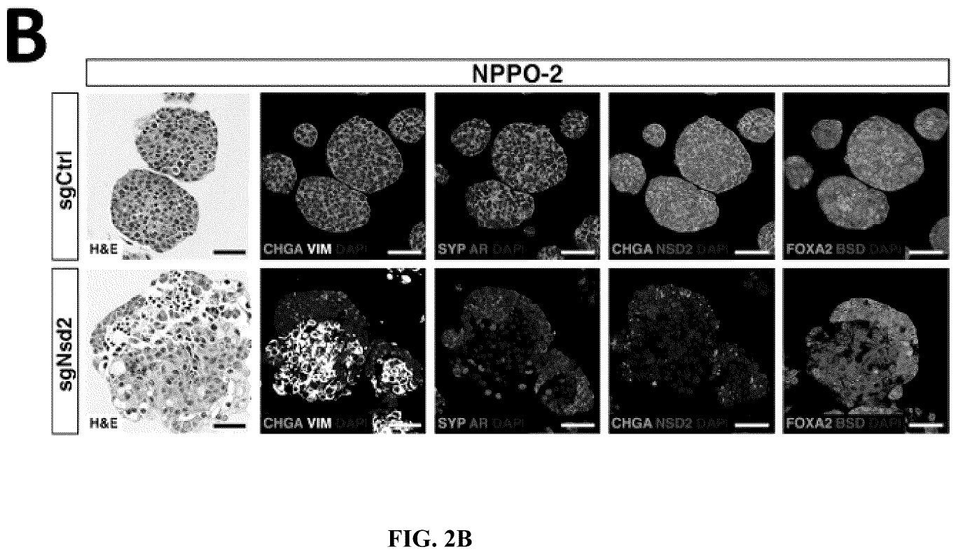

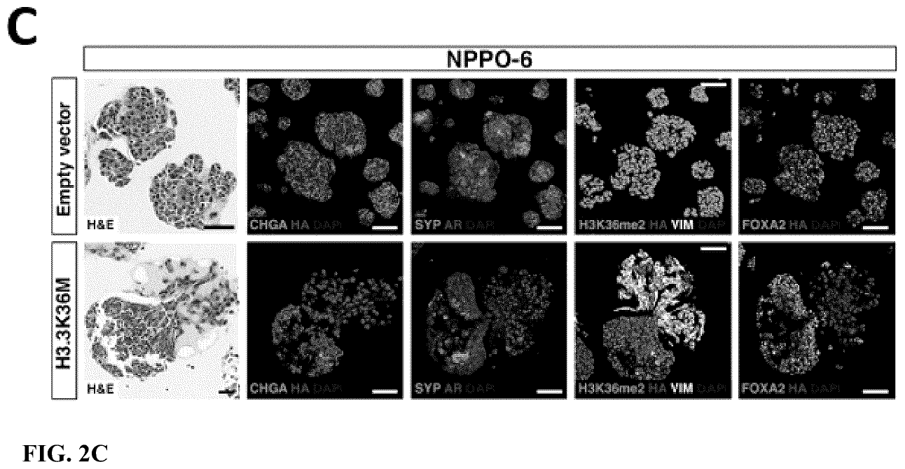

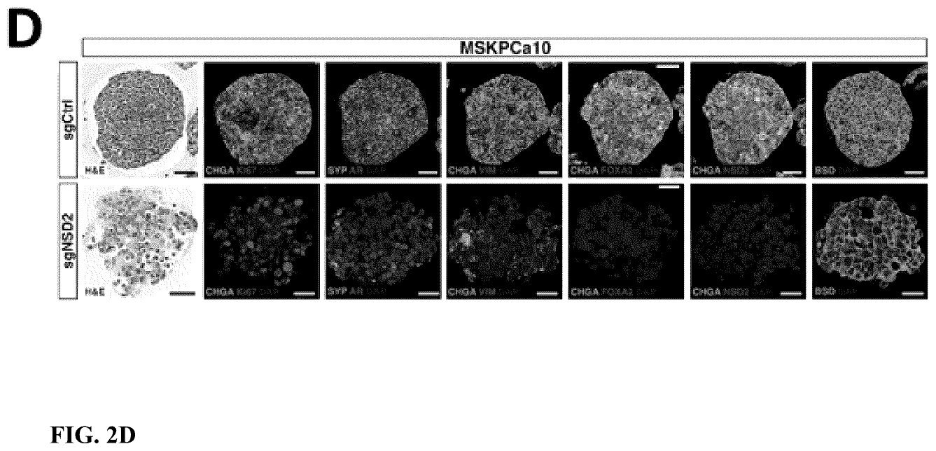

To determine the role of H3K36me2 in neuroendocrine differentiation, we used CRISPR/Cas9 targeting of NSD2, which encodes a histone methyltransferase that generates H3K36me1 and H3K36me2 marks. Following CRISPR-mediated knock-out of NSD2, histological alterations and numerous AR-positive cells were observed that were negative for NE markers in the in NPPO-2 and NPPO-1NE organoid lines, consistent with conversion of neuroendocrine cells to AR-positive adenocarcinoma cells following NSD2 loss (FIGS. 2A-B), however, no effects were observed in the NPPO-4 and NPPO-6 lines; the NPPO-1NE line is a subline of NPPO-1 in which the neuroendocrine component of NPPO-1 was isolated by flow cytometry, resulting in a pure neuroendocrine organoid line. At 14 days, alterations were observed in organoid morphology as well as marker expression (FIGS. 2A-B). The control NPPO-1NE organoids formed compact organoids with smooth edges, whereas the knock-out NPPO-1NE organoids formed cystic structures with palm tree-like protrusions. Immunofluorescence staining showed that the knock-out organoids contained many AR-positive and Vimentin-positive mesenchymal cells, in contrast with the nearly homogeneous AR-negative and Chga/Syp-positive neuroendocrine cells in the control organoids (~1% of Vim-positive cells). These findings suggest that many of the small cell-like neuroendocrine cells in NPPO-1NE organoids converted to AR-positive adenocarcinoma cells following NSD2 knock-out. Similar findings were observed with NPPO-2 organoids, which also developed mesenchymal AR-positive cells after NSD2 knock-out (FIGS. 2A-B).

### Depletion of H3K36Me2 Results in Loss of Neuroendocrine Differentiation

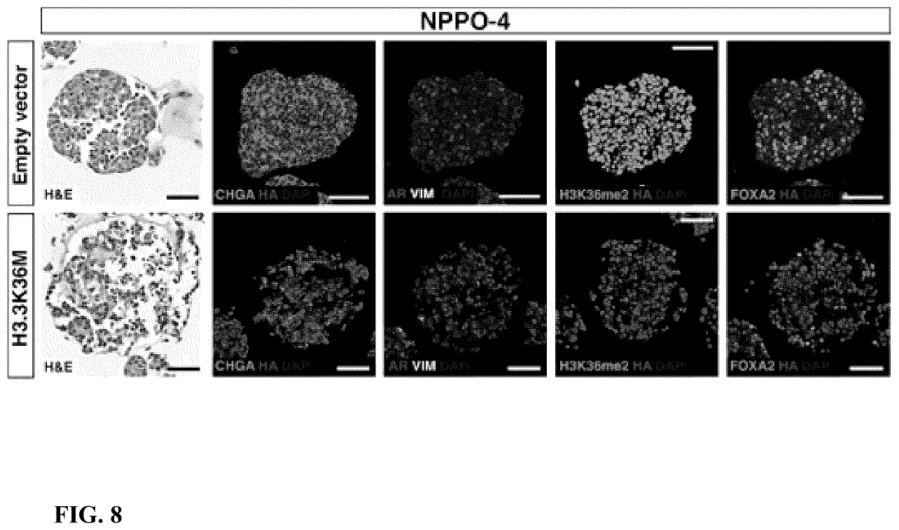

Since NSD2 is known to have functions independent of its role as a histone methyltransferase, we also tested whether depletion of H3K36me2 would also affect neuroendocrine differentiation. For this purpose, lentiviral infection was used to express the H3.3K36M mutation in organoids. The H3.3K36M mutant has a dominant-negative effect on NSD family and SETD2 methyltransferases and results in depletion of H3K36me2 and H3K36me3⁴⁹⁻⁵¹. We found that lentiviral H3.3K36M expression led to lethality of NPPO-1NE and NPPO-2 organoids, and consequently examined its effects on the NPPO-4 and NPPO-6 lines (FIGS. 2C, FIGS. 6C, and 8). H3.3K36M expression in NPPO-4 organoids led to dramatic reduction of neuroendocrine marker (Chga and Foxa2) expression, but did not result in the appearance of Vim-positive mesenchymal cells; no changes were observed in control organoids. However, H3.3K36M expression in NPPO-6 organoids led to loss of neuroendocrine marker expression as well as abundant AR-positive and Vim-positive mesenchymal cells, consistent with a transition from amphicrine neuroendocrine to a mesenchymal adenocarcinoma state (FIG. 2C and FIG. 8A).

To confirm these findings in human CRPC-NE, the MSKPCa10 organoid line was utilized, which is mutant for TP53 and lacks Rb1 expression⁵². CRISPR-mediated targeting of NSD2 resulted in loss of CHGA- and SYP-positive neuroendocrine cells and gain of AR expression, as well as histopathological changes with a decreased nucleus to cytoplasm (N:C) ratio (FIG. 2D).

### Inhibition of H3K36Me2 Increases Response to Anti-Androgen Treatment

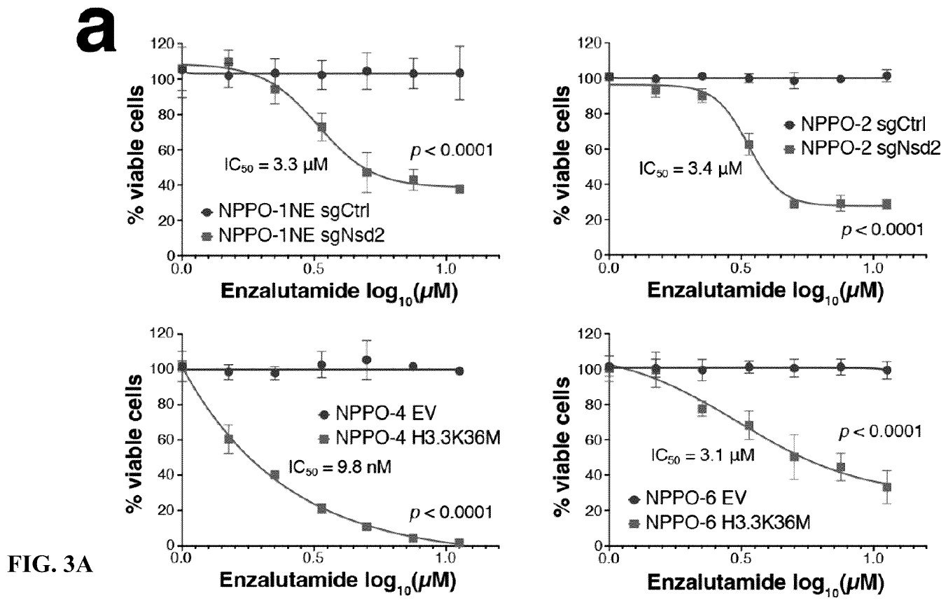

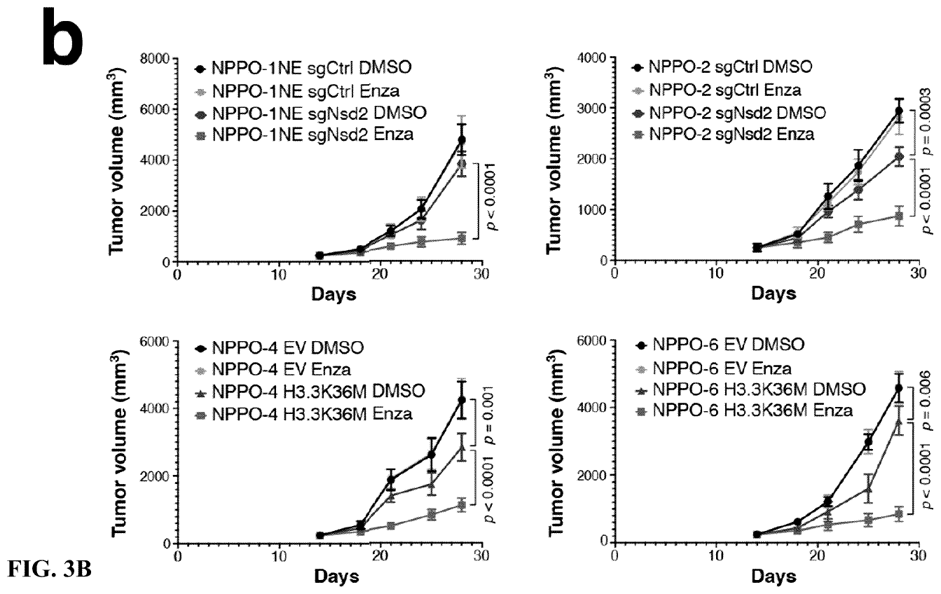

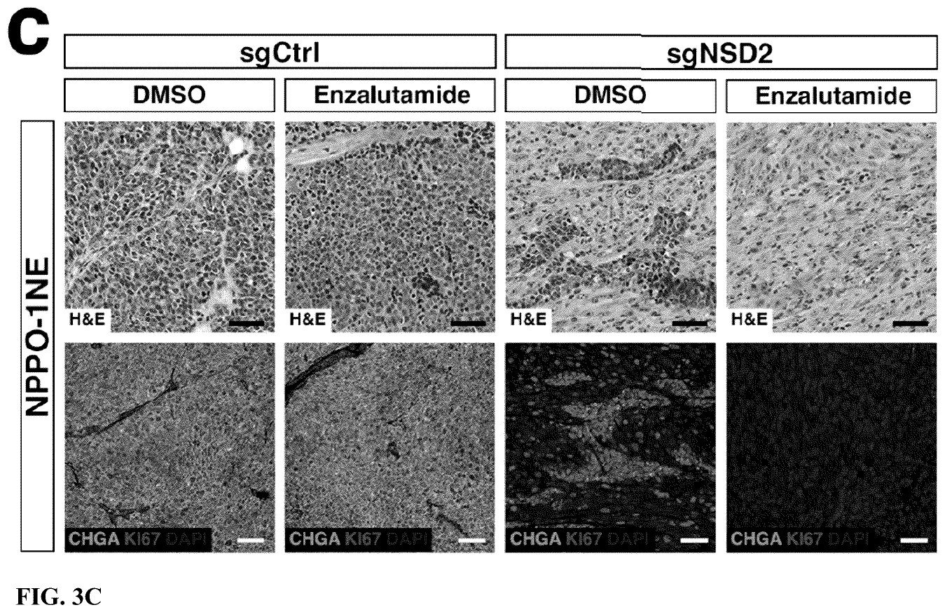

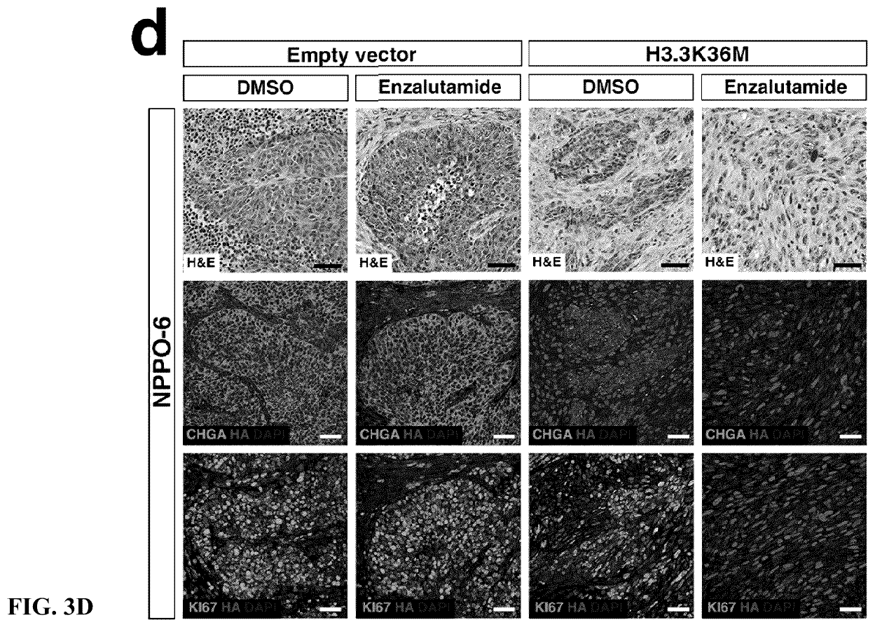

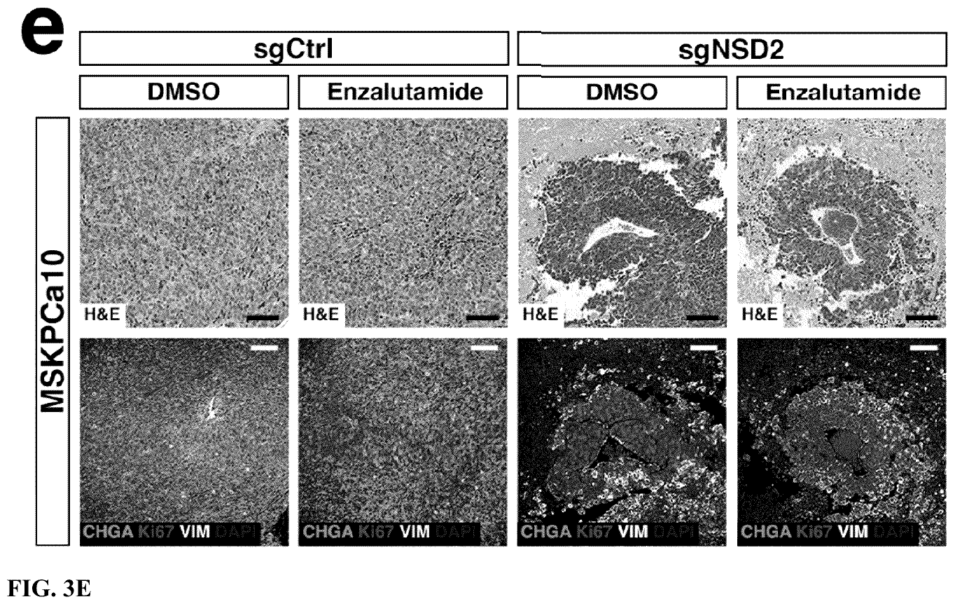

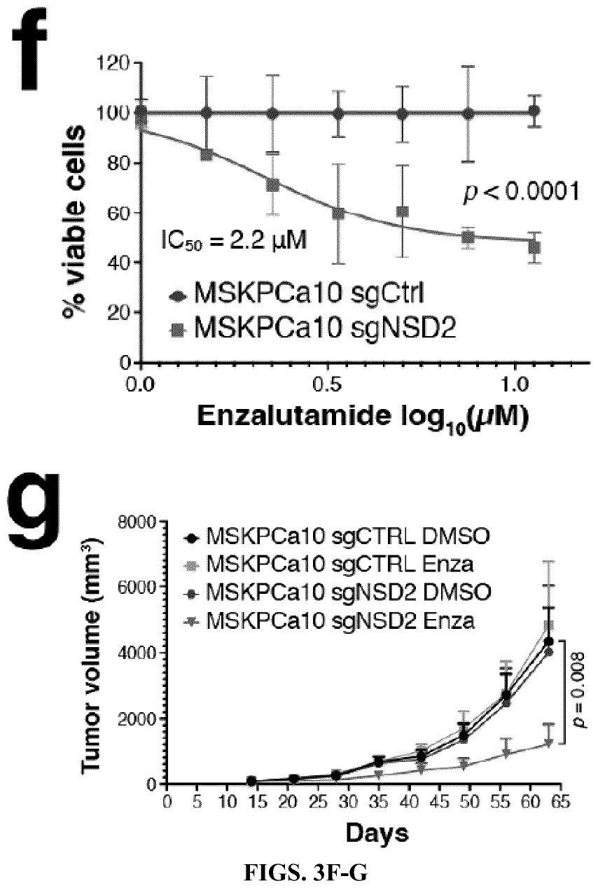

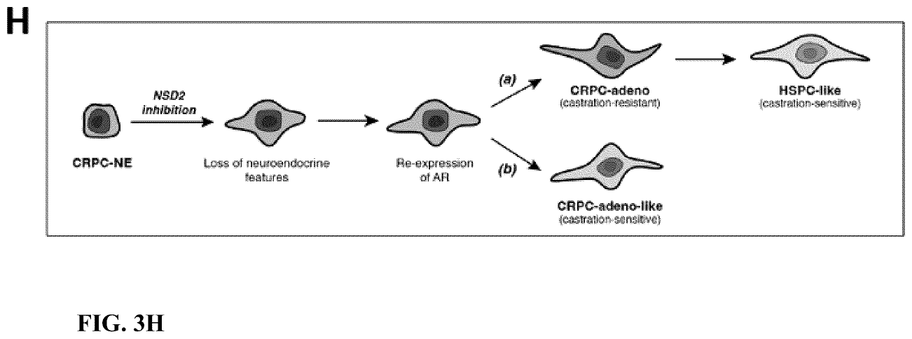

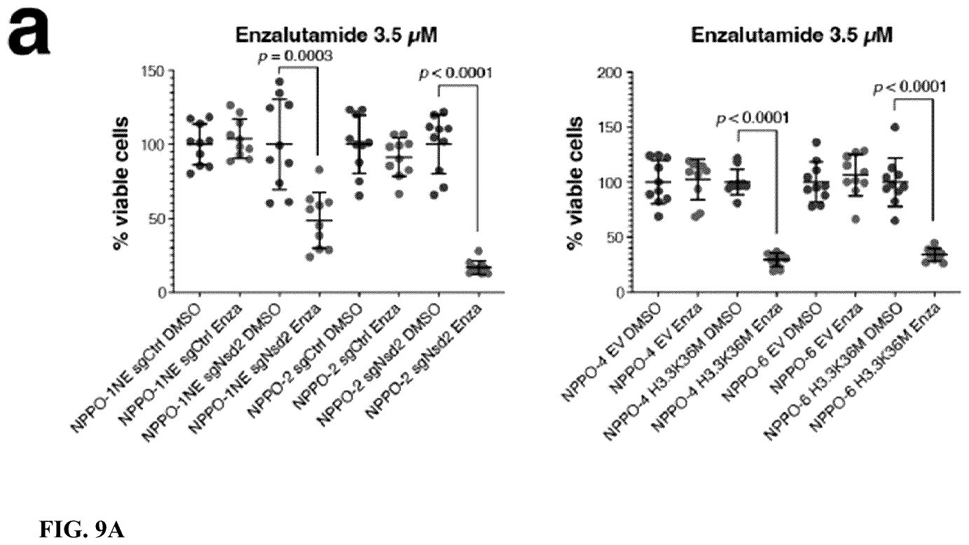

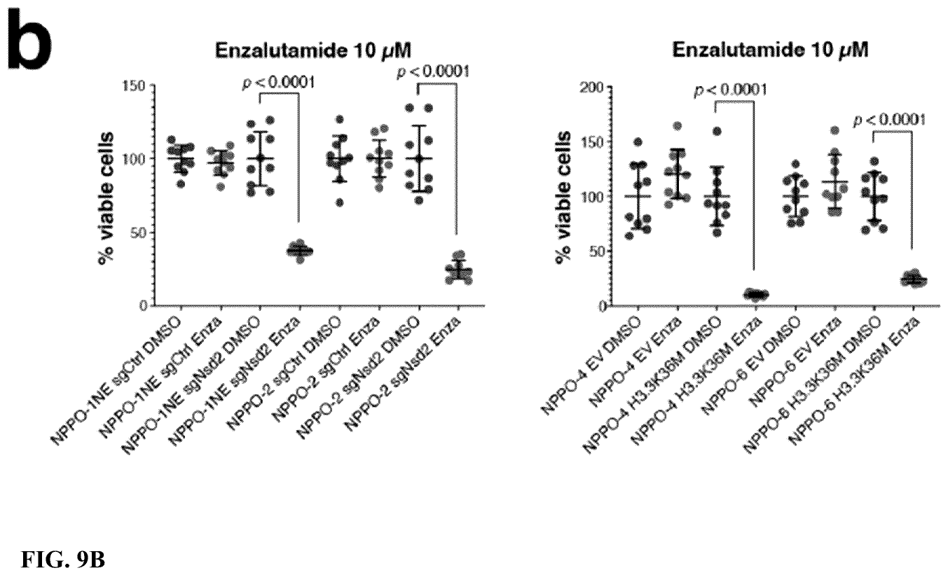

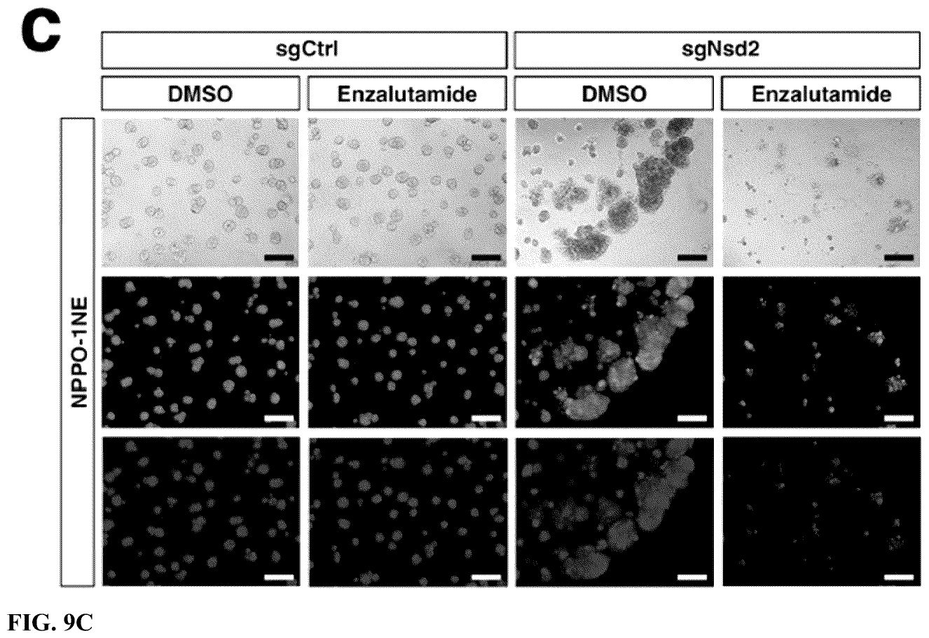

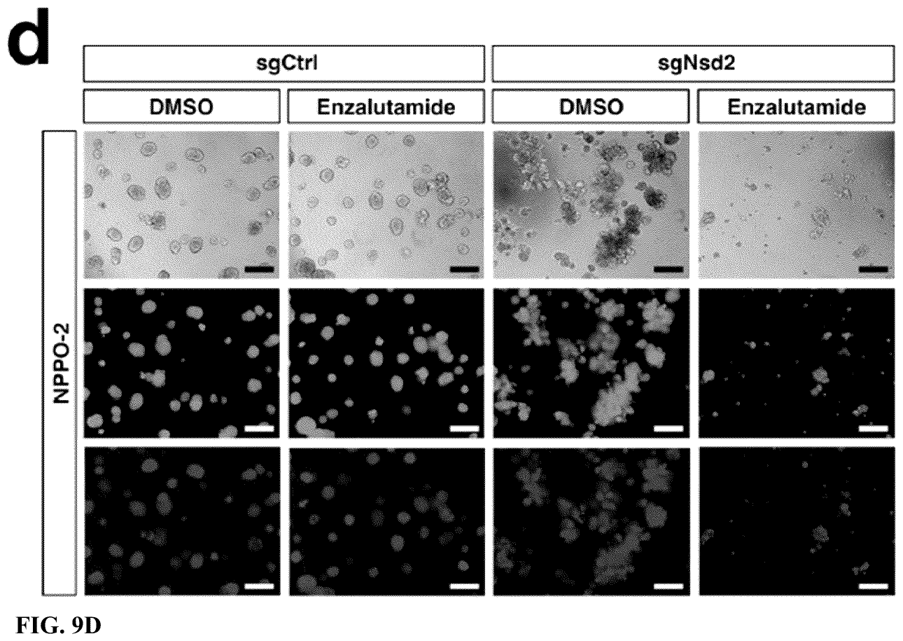

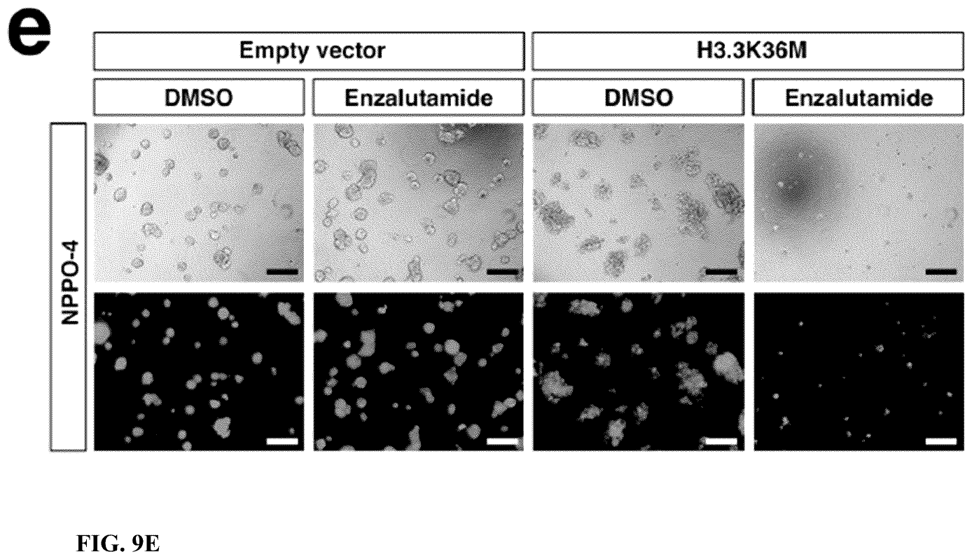

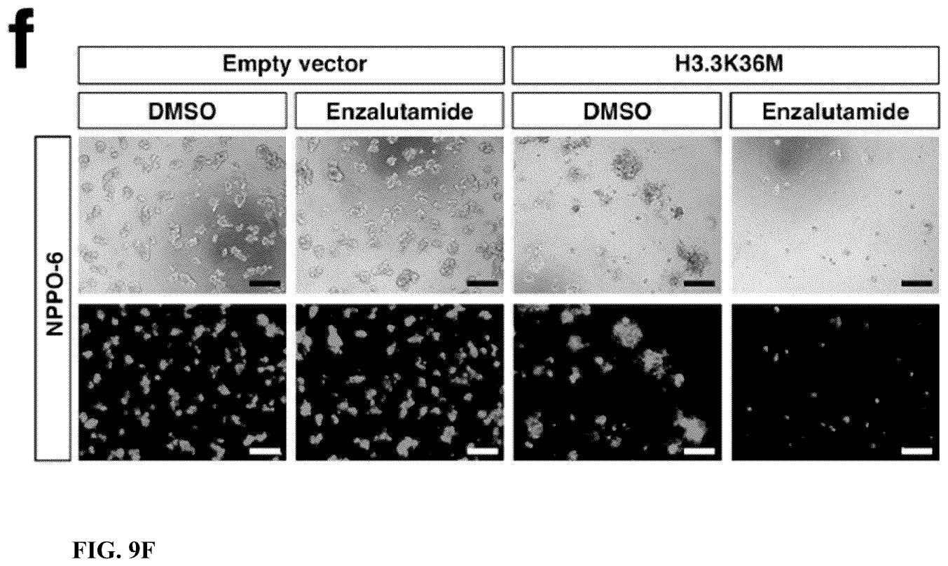

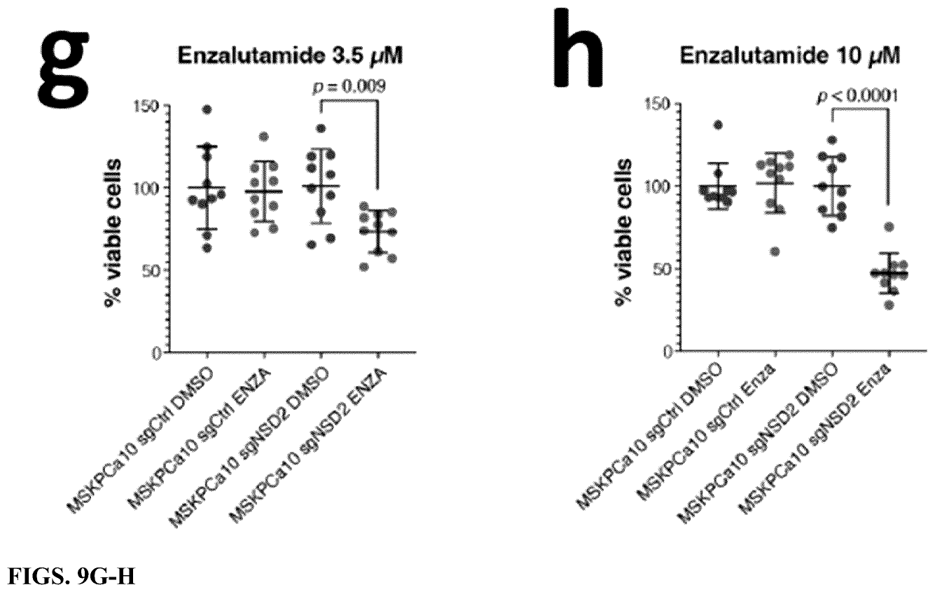

Since NEPC is resistant to treatment with second-generation anti-androgens such as enzalutamide and abiraterone, it was next investigated whether NSD2 knock-out or H3.3K36M expression would result in increased response to enzalutamide. We first generated dose-response curves for enzalutamide treatment of organoids, and found that control organoids were highly resistant to enzalutamide, whereas NSD2 knock-out or H3.3K36M expressing organoids were much more responsive, resulting in typical IC50 values of approximately 3 μM (FIG. 3A, FIG. 9A-F). Next, organoids in culture were treated with 10 μM or 3.5 μM enzalutamide (FIG. 9A-B). As expected, enzalutamide did not affect growth of control organoids, even for the NPPO-4 and NPPO-6 lines that had low levels of AR expression. However, both 10 μM and 3.5 μM enzalutamide could significantly reduce organoid growth of all four lines tested after NSD2 knock-out or H3.3K36M expression.

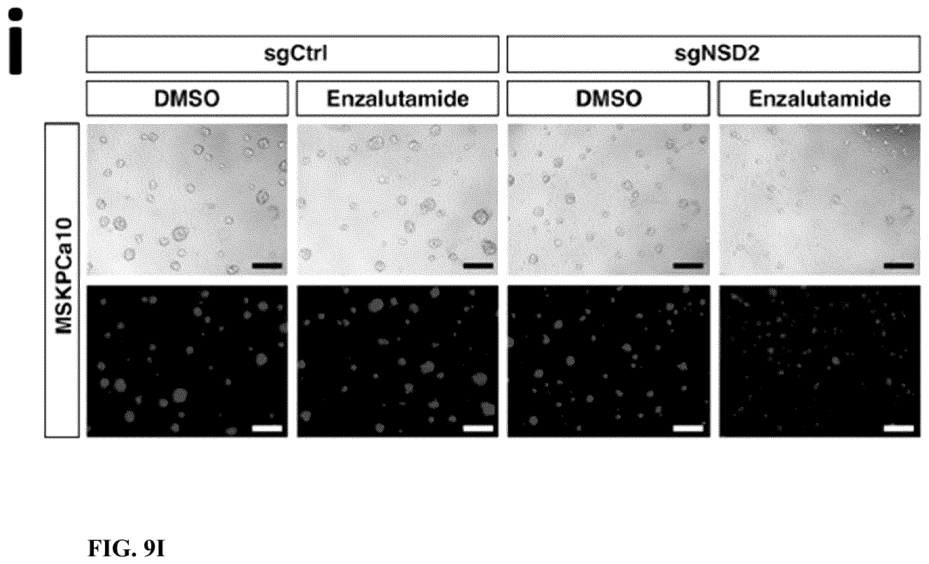

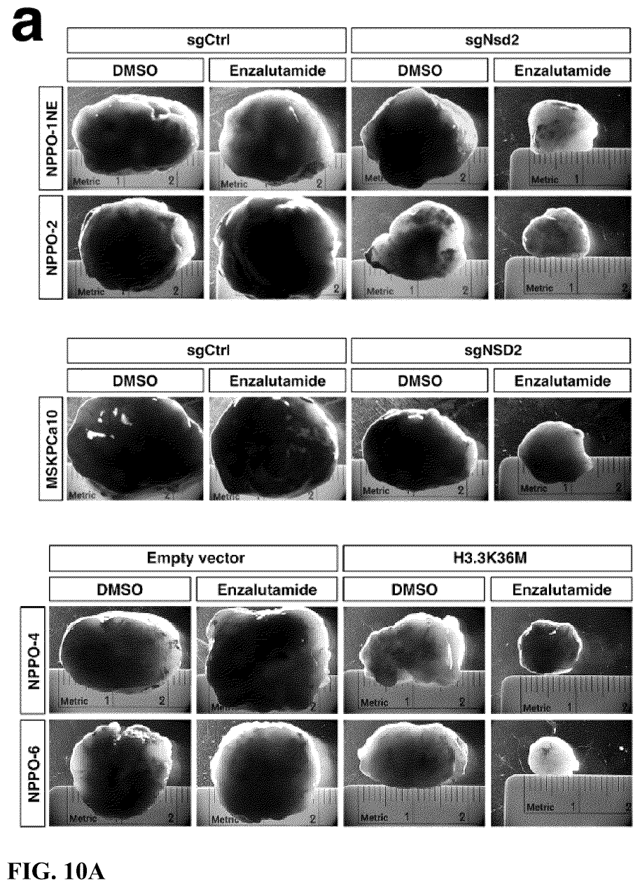

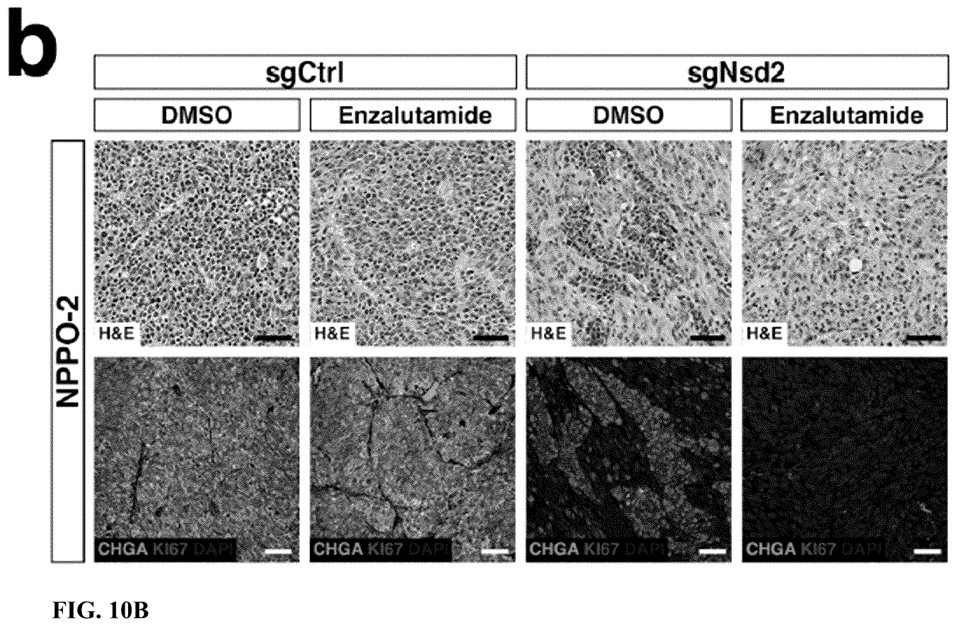

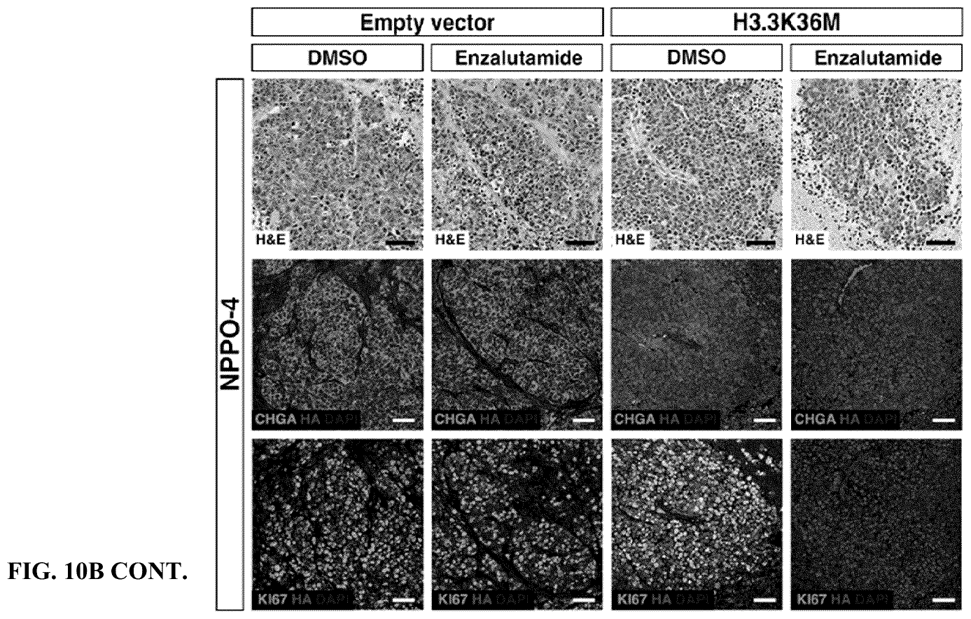

To confirm these findings in vivo, subcutaneous allografting of transfected organoids in immunodeficient NOD/SCID mice Followed by treatment of host mice with enzalutamide or DMSO as a control after tumors reached approximately 200-250 mm³ in size at two weeks after grafting. Compared to controls, enzalutamide treatment significantly reduced the growth of NSD2 knock-out NPPO-1NE and NPPO-2 grafts as well as H3.3K36M-expressing NPPO-4 and NPPO-4 grafts (FIG. 3B and FIG. 10A). Analysis of graft sections by H & E staining and immunofluorescence showed that a substantial number of neuroendocrine marker-expressing cells remained in NSD2 knock-out or H3.3K36M expressing organoids after DMSO treatment, but no neuroendocrine cells could be detected after enzalutamide treatment (FIGS. 3C, 3D, and 10B). Moreover, there was no Ki67 staining observed in the organoids with NSD2 knock-out or H3.3K36M expression together with enzalutamide treatment, indicating that this combination treatment could abrogate tumor growth. Analysis of graft sections by H & E staining and immunofluorescence showed that NSD2 inhibition resulted in loss of NE marker expression, decreased Ki67 expression, and gain of adenocarcinoma phenotypes (FIGS. 3C-D, FIG. 10B). Similar results were observed with NSD2 targeting in human MSKPCa10 organoids, which displayed decreased growth after enzalutamide treatment of organoids and xenografts (FIGS. 3E-G, FIGS. 9H, I, and FIG. 10A). These results suggest that the reversal of neuroendocrine differentiation by NSD2 inactivation or H3.3K36M expression renders the resulting CRPC-NE tumor cells more responsive to anti-androgen treatment. These results also indicate that NSD2 inhibition renders CRPC-NE more responsive to enzalutamide.

### DISCUSSION

The findings described herein demonstrate that NSD2 plays a critical role in maintenance of neuroendocrine states and castration-resistance in CRPC-NE. In particular, loss of NSD2 results in reversal of neuroendocrine differentiation and alteration of the lineage plasticity of CRPC-NE. These activities of NSD2 in lineage plasticity are correlated with epigenetic reprogramming and transcriptional activation of key CRPC-NE regulators including FOXA2 and ONECUT2^{58,59}.

Importantly, inhibition of NSD2 in CRPC-NE also results in re-expression of AR, but this NSD2-deficient state is sensitive to the AR inhibitor enzalutamide, unlike CRPC-adeno which is castration-resistant. Thus, we can envision two conceptual models for how NSD2 loss might result in restoration of enzalutamide sensitivity (FIG. 3H). One possibility is NSD2 has a unitary role in modulating lineage plasticity in CRPC, such that NSD2 loss would fully revert CRPC-NE back to an AR-positive cellular state that resembles hormone-sensitive prostate cancer (HSPC). Alternatively, loss of NSD2 might revert CRPC-NE to an adenocarcinoma state that has acquired sensitivity to AR inhibitors, due to an activity of NSD2 in maintaining castration-resistance that is independent of its role in lineage plasticity.

With respect to this second model, NSD2 might facilitate castration-resistance through a protein-protein interaction between NSD2 and AR that is mediated by the HMG domain of NSD2 and thereby alters AR transcriptional activity⁶⁰. Thus, NSD2 loss could affect AR binding properties, consistent with the observed alterations in the AR cistrome following NSD2 inhibition. If this model is correct, NSD2 may have a broader requirement in castration-resistance for at least a subset of non-neuroendocrine mCRPC with higher NSD2 levels, which remains to be evaluated. Furthermore, NSD2 has been previously implicated in promoting metastasis of prostate cancer^{46,61,62}, and may thereby represent a functional link between lineage plasticity, castration resistance, and metastasis in mCRPC.

The functions of NSD2 in prostate cancer are consistent with a general role of H3K36me2 marks and NSD histone methyltransferases in promoting lineage plasticity and metastasis in a range of tumor types⁶³. Notably, NSD2 is activated by a t(4,14) translocation in a major subtype of multiple myeloma⁶⁴, and its gain-of-function mutations are frequently observed in pediatric acute lymphoblastic leukemia⁶⁵. The related genes NSD1 and NSD3 have also been shown to be key drivers of head and neck cancer and squamous cell lung cancer, respectively⁶⁶⁻⁶⁸. Furthermore, NSD2 may be therapeutically targetable, as a small molecule inhibitor of NSD2 is being tested in an early-phase clinical trial (NCT05651932) for relapsed t(4,14) translocation-positive multiple myeloma. In combination with the findings disclosed herein, these observations suggest that NSD2 and its relatives play much broader roles in tumor plasticity than previously suspected and represent attractive therapeutic targets.

### Methods

### Mouse Procedures

The Nkx3.1CreERT2/+; Ptenflox/flox; TrpP53flox/flox; Rosa26-EYFP (NPp53) mice were maintained on a mixed C57BL/6-129Sv background and have been previously described⁷. Tamoxifen induction was performed in mice at 3-5 months of age by oral delivery of tamoxifen (Sigma; 100 mg/kg/day in corn oil) for 4 consecutive days as described previously⁶⁹. The survival time of tumor-bearing mice in this study ranged from 228 to 435 days after tamoxifen induction. All procedures followed protocols approved by the Institutional Animal Care and Use Committee (IACUC) at Columbia University Medical Center.

### Establishment and Maintenance of Mouse Prostate Organoids

Tumor tissues from NPp53 mice were cut into two pieces, with half fixed in 10% formalin for paraffin embedding, and the other half used for organoid establishment. Tissues were minced with scissors in 0.2% collagenase IV (Thermo Fisher Scientific 17104019) and incubated at 37° C. for 30 min, followed by neutralization with 1:10 Hank's buffer (STEMCELL Technologies 37150) supplemented with 10 μM Y-27632 (STEMCELL Technologies) and 5% charcoal-stripped fetal bovine serum (CS-FBS; Gemini 100-119). After centrifugation at 1000 rpm for 10 min, pellets were incubated with prewarmed TrypLE (Thermo Fisher Scientific 12605010) at 37° C. for 10 min. The cell suspension was then neutralized 1:10 with PBS, passed through a 100 μM cell strainer (Corning 352360), and spun down at 1000 rpm for 10 min. Prior to plating, cell numbers were counted in a TC20 automated cell counter (Bio-Rad).

Cells were resuspended in organoid culture media supplemented with 10 μM Y-27632, 10 μM A83-01 (Tocris 2939), and 5% Matrigel (Corning 354234) and plated at a seeding density of approximately 50,000 cells/well in 96-well ultra-low attachment microplates (Corning 3474). Organoid culture medium consisted of hepatocyte culture medium (Corning 355056), 5% CS-FBS, 1× GlutaMAX™ supplement (Thermo Fisher Scientific 35050061), 5 ng/ml EGF, 100 μg/ml primocin (Invivogen ant-pm-1) and 100 nM dihydrotestosterone (DHT), as previously described⁷⁰. For heterogeneous NE organoids, such as NPPO-1 and NPPO-5, organoid culture medium was replaced every 4 days. For homogeneous NE organoids, such as NPPO-1NE, NPPO-2, NPPO-4 and NPPO-6, we used NE organoid culture medium, which was identical to the organoid culture medium except that no EGF was added; the medium was replaced every 4 days.

For passaging, organoids were collected by centrifugation at 1000 rpm for 1 min, followed by addition of 1 ml pre-warmed TrypLE for 10 min at 37° C. for cell dissociation. After neutralization with 10 ml PBS, cells were spun down and counted, with approximately 50,000 cells plated per well in 96-well ultra-low attachment microplates (Corning 3474). To generate cryopreserved stocks, organoids were frozen in 90% CS-FBS and 10% DMSO and stored in liquid nitrogen. We considered NE organoid lines to be successfully established when they could be stably passaged, cryopreserved, and recovered without loss of NE phenotypes.

### Human Prostate Tumor Organoids

MSKPCa10 organoids have been previously described⁵². Human prostate tumor organoids were maintained in 60% Matrigel and 40% human NE culture medium, which was replaced every other day. Human NE culture medium consisted of hepatocyte culture medium, 5% CS-FBS, 1× GlutaMAX™, 5 ng/ml EGF, 100 μg/ml primocin and 10 nM DHT.

### Hematoxylin-Eosin Staining

For tissue processing and embedding, organoids were fixed in 10% formalin (Thermo Fisher Scientific, SF100-4) for 1 h, washed once with PBS, placed in rat tail collagen I (Corning 354249) and incubated at 37° C. for 30 min. The collagen button was then put into a biopsy cassette (Thermo Fisher Scientific 15182705E) and fixed in 10% formalin for 24 h. After replacing the formalin with 70% ethanol, the cassettes were put into an automated tissue processor for tissue processing and embedding.

Paraffin-embedded blocks were sectioned into 5 μm sections using microtome and dried by onto microscope slides at RT. Paraffin sections were baked at 65° C. for 15 minutes prior to deparaffinization with three changes of xylene, 5 min each. The slides were hydrated through 100%, 95%, and 95% ethanol, 5 min each, rinsed in tap water for 2 min, and incubated in Gill™ Hematoxylin 3 (Epredia 72611) for 3-30 min. Slides were rinsed in tap water and dipped 3-5 times in 0.5% Acid Alcohol (Leica Biosystems 3803651), followed by rinsing in tap water and bluing with Scott's Tap Water (Electron Microscopy Sciences 2607007) for 5 min. After rinsing in tap water, slides were incubated in 95% ethanol for 5 min and counterstained with Eosin (Poly Scientific S1761GL) for 1-3 min. Slides were passed through 70%, 95%, and 100% (3×) ethanol, rinsed 3 times in xylene, and coverslipped with mounting medium (StatLab, MMC0126). Images were captured using an Olympus BX 61 VS Slide Scanner.

### Immunofluorescence Staining

Paraffin sections (5 μm) were dried onto microscope slides at RT, incubated at 65° C. for 15 min prior to deparaffinization through three changes of xylene (5 min each), hydrated in 100%, 95%, 95%, 75% ethanol (5 min each), and washed in tap water for 2 min. Antigen retrieval was performed by immersion in boiling citrate buffer (pH 6) for 10 min, cooling to RT for 30 min, and incubation in Milli-Q water at RT for 10 min. Sections were permeabilized with 0.5% Triton X-100 in PBS (MilliporeSigma 11332481001) for 10 min and blocked in 10% goat serum for 1 hr. Diluted primary antibodies (FIG. 26) were added to sections and incubated overnight at 4° C. The next day, sections were washed with PBS three times, 15 min each, and incubated with secondary antibodies at RT for 1 hr. After washing with PBS three times, 15 min each, nuclei were stained with DAPI (Thermo Fisher Scientific D1306) for 5 min. Slides were washed with PBS and mounted with VECTASHIELD® Antifade Mounting Medium (Vector Laboratories H-1200-10). Images were captured using a Leica TCS SP5 Confocal Laser Scanning Microscope (Leica Microsystems) using Leica Application Suite Advanced Fluorescence (LAS AF).

Isolation of NE and Non-NE Cells from NPPO-1 Organoids

To sort NE and non-NE populations, NPPO-1 organoids at passage 2 were incubated with prewarmed TrypLE at 37° C. for 10 min, neutralized with 1:10 PBS and 5% CS-FBS, spun at 1000 rpm for 1 min, resuspended with PBS, and dissociated into single cells by gentle pipetting. The cells were filtered three times through a 40 μm cell strainer (Corning 431750), spun at 1000 rpm for 5 min, and resuspended with PBS and 2% CS-FBS. After filtering through a Falcon tube with 35 μm strainer cap (Corning 352235), cell numbers were counted in a TC20 automated cell counter, and the volume adjusted to a final cell concentration of 5,000 cells/μl.

Flow sorting was performed on an BD Influx™ cell sorter (BD Biosciences) in the Flow Cytometry Core of the Columbia Center for Translational Immunology (CCTI). Gating by forward scatter (FSC) and side scatter (SSC) was used to exclude debris, and doublets were excluded by gating on trigger pulse width against FSC height. Individual NE and non-NE tumor cells were sorted based on scatter parameters. As NE tumor cells have less internal complexity (granularity) than non-NE tumor cells and exhibit lower intensity SSC. Flow sorting data were collected and analyzed using BD FACS™ Software (BD Biosciences, version 1.2.0.142). Cell purity was assessed following flow sorting by single-cell RNA sequencing.

### Lineage-Tracing in Organoids

Flow-sorted NE cells from NPPO-1 organoids were maintained in NE organoid culture medium with 5% Matrigel. Half of the flow-sorted non-NE cells were used for scRNA-seq (Columbia University Single Cell Analysis Core), immediately after sorting. The other non-NE cells were transfected with H2B-RFP (Addgene 26001) lentivirus. Approximately 70% of non-NE cells were labeled with H2B-RFP at 3 days after transfection, in the absence of antibiotic selection. On day 7, the cells were digested with TrypLE and passed three times through a 40 μm cell strainer to ensure a single cell suspension, following by cell counting. For co-culture, H2B-RFP labeled non-NE cells were seeded together with NE cells at a ratio of 2:3 in 96-well ultra-low attachment microplates; as a control, H2B-RFP labeled non-NE cells were seeded alone. The resulting organoids were cultured in NE organoid culture medium with 5% Matrigel, and analyzed at passage 4 by immunostaining and scRNA-seq.

### Imaging of Histone and DNA Modifications

To screen for differential expression of histone modifications between NE and non-NE tumor cells in NPPO-1 organoids, we performed immunofluorescence staining with antibodies detecting the NE markers Synaptophysin (SYP) or Chromogranin A (CHGA), the non-NE marker Vimentin (VIM), and various histone/DNA modifications (FIG. 26). Images were captured using a Leica TCS SP5 confocal laser scanning microscope and images acquired with Leica Application Suite Advanced Fluorescence (LAS AF). Fluorescence intensity was measured using ImageJ (NIH; version 1.52K) using three parameters: area, integrated density (IntDen), and mean gray value. Background was measured from a region that had no fluorescence on the same image. Intensity was calculated using the formula: Intensity=IntDen−(Area×Mean fluorescence of background readings) using measurements collected from three independent organoids, and graphed using Prism 9 (GraphPad software version 9.3.1). An unpaired t-test was used to compare means, and P values calculated from a two-tailed t test.

### Lentivirus Production and Transfection

Lentiviruses were generated by transfection of 293T cells with the indicated expression plasmid and the psPAX2 (Addgene 12260) and pVSVG (Addgene 14888) packaging vectors at a ratio of 4:2:3, respectively. Viral supernatants were collected at 48, 72, and 96 h after transfection, filtered, and concentrated using Lenti-X Concentrator (Takara Bio 631232). For CRISPR/Cas9 gene knockout, we used the lentiCas9-blast plasmid (Addgene 52962) and a custom vector for sgRNA (U6-sgRNA-EFS-Puro-P2A-TurboRFP in a pLL3-based lentiviral backbone; gift from Scott Lowe). For sgRNA design, the CRISPick platform (BROAD institute) was used. HA-tagged H3.3K36M was overexpressed in the pCDH vector (gift from David Allis). The sgRNas used in the experiment are: sgControl, 5′ GAG ATA AGC ATT ATA ATT CCT 3′ (SEQ ID NO: 1); sgNsd2 (mouse): 5′ TCA GGG TCT CAC AAT TGG GC 3′ (SEQ ID NO: 2); sgNSD2 (human): 5′ GCA CCA GCT CAC GTT GAC GT 3′ (SEQ ID NO: 3).

For transfection, organoids were incubated with high-titer lentivirus in culture medium supplemented with 8 μg/ml polybrene (MilliporeSigma TR-1003). Medium containing virus was removed on the next day and switched to normal organoid medium with Matrigel. Selection with appropriate antibiotics was performed at 3 days after transfection for 7-14 days.

### Flow Sorting of Transfected Cells

For CRISPR/Cas9-mediated gene knockout experiments, organoids were transfected with the lentiCas9-blast plasmid and blasticidin selection to establish stable lines. A custom vector for the sgRNA lentivirus carrying a TurboRFP reporter and puromycin antibiotic was then transfected into the Cas9-expressing organoids. After 14 days of antibiotic selection, we performed fluorescence-activated cell sorting (FACS) on the PE channel to sort RFP-positive (RFP+) cells on an BD Influx™ cell sorter, as described above. Approximately 1×10⁶ RFP+ cells were collected in 0.04% BSA in PBS (Miltenyi Biotec 130-091-376) for single-nuclei isolation and multi-ome ATAC/sequencing (10× Genomics). Additional collected RFP+ cells were used for organoid culture.

For experiments in which mouse NE tumor organoids were transfected with a HA-tagged H3.3K36M lentiviral vector, we performed flow cytometry at 14 days after antibiotic selection to determine the purity of the culture. Organoids were dissociated into single cells, and resuspended in 100 μl 4% paraformaldehyde/1×10⁶ cells to fix for 15 min at room temperature (RT). The fixed cells were neutralized with 1 ml PBS, washed once with PBS, and resuspended in 0.5 ml PBS. The cells were permeabilized for 10 min by addition of 0.5 ml 1% Triton-X100 with gentle vortexing to a final concentration of 0.5% Triton-X100. Cells were washed in 10 ml PBS, and resuspended with 100 μl Cy5.5® Conjugated mouse anti-HA-Tag antibody (Clone 6E2, Cell Signaling Technology 62145) diluted 1:50 in 0.5% BSA in PBS. Cells were incubated with antibody for 1 hour in the dark at RT, washed twice in 0.5% BSA, resuspended in 300 μl 0.5% BSA, and filtered through a Falcon™ Tube with 35 μm cell strainer cap. Flow cytometry was performed on the APC channel using a FACSCanto II Flow Cytometer (BD Bioscience) as described above, using Cy5.5 positive events to determine the percentage of HA-Tag+ cells. Flow data were analyzed using FlowJo (BD, version 10.8.2). The same batch of cells was collected in 0.04% BSA in PBS for single nuclei isolation and multiome snATAC/snRNA-sequencing.

### Drug Treatment of Organoids

To generate drug response curves, organoids were digested with TrypLE for 10 min at 37° C., neutralized with PBS, gently dissociated into single cells, and passed through a 100 μm cell strainer. Cells were resuspended in 5% Matrigel in NE organoid culture medium lacking DHT and plated in triplicate at a seeding density of 5,000 cells/well in 96-well ultra-low attachment microplates. The next day, 7 doses of enzalutamide in 0.1% DMSO were dispensed at 1.5-fold dilution from 1 μM to 11.25 μM. Cell viability was assayed after five days using CellTiter-Glo 3D (Promega G9683), with luminescence was measured by a GloMax® Explorer multimode plate reader (Promega). Background luminescence was measured in medium without cells. The percentage of viable cells was calculated by the formula:

$$viable cell \% = \frac{Experimental value - background reading}{Vehicle value - background reading} \times 100$$

Drug response curves were generated by nonlinear regression using the percentage of viable cells against logarithm of drug concentrations using Graphpad Prism 9. IC₅₀ values were calculated by the equation log (inhibitor) versus response (variable slope, four parameters). Two-way ANOVA was used to compare dose-response curves.

Similar methods were used to determine response of mouse or human organoids to defined doses of enzalutamide, using 5,000 cells/well (mouse) or 10,000 cells/well (human). The percentage of viable cells from different treatment groups were graphed using Prism 9. Unpaired t tests were used to compare means between two groups. All experiments were repeated independently at least three times with consistent results observed.

### Grafting Assays

To generate tumors in vivo, mouse NPp53 or human prostate organoids were grafted into 6-8 week old NOD/SCID male mice (NOD.CB17-Prkdcscid/J, Jackson Laboratory 001303). NOD/SCID mice underwent surgical castration at 7 days prior to grafting. For mouse grafts, 1×106 dissociated organoid cells in 100 μl hepatocyte culture medium and 5% Matrigel were injected subcutaneously into the flank using a 1 ml syringe with 25G needle (BD 305122). For human grafts, 3×106 dissociated cells in 100 μl 60% Matrigel and 40% hepatocyte culture medium were injected. Tumor sizes were measured with a digital caliper. In each cohort, 20-24 mice whose tumor volume had reached ~250 mm³ (mouse grafts) or ~80 mm³ (human grafts) at week two after grafting received either 10 mg/kg enzalutamide (TargetMol T6002) or 0.5% DMSO (MilliporeSigma D2650) by daily gavage via 20G needle (Roboz FN-7910) for 14 days (mouse grafts) or 56 days (human grafts). Enzalutamide or DMSO was suspended in 1% carboxymethylcellulose (MilliporeSigma 419281) and 0.1% Tween 80 (MilliporeSigma P4780) in distilled water. At the end of drug treatment, tumors were harvested and imaged under a stereo microscope (Olympus SZX16 with DP71 digital camera) using Olympus DP Controller 3.3.1.292 (Olympus). Tumor tissues were fixed in 10% formalin for 24-48 h and processed in the Columbia Molecular Pathology Core Facility. Tumor volumes were calculated using the formula:

$$V = {w}^{2} \cdot \frac{l}{2}$$

Tumor growth curves were plotted using Prism 9. Unpaired t tests were used to compare means between two groups, and P values were from two-tailed t tests.

### REFERENCES FOR EXAMPLE 1

1. Zou, M., Toivanen, R., Mitrofanova, A., Floch, N., Hayati, S., Sun, Y., Le Magnen, C., Chester, D., Mostaghel, E. A., Califano, A., Rubin, M. A., Shen, M. M. and Abate-Shen, C. (2017). Transdifferentiation as a mechanism of treatment resistance in a mouse model of castration-resistant prostate cancer. Cancer Discov. 7:736-749. PMID: 28411207. PMCID: 5501744.
2. Chua, C. W., Shibata, M., Lei, M., Toivanen, R., Barlow, L. J., Bergren, S. K., Badani, K. K., McKiernan, J. M., Benson, M. C., Hibshoosh, H. and Shen, M. M. (2014). Single luminal epithelial progenitors can generate prostate organoids in culture. Nat. Cell Biol. 16:951-961. PMID: 25241035. PMCID: 4183706.
3. Gao, D., Vela, I., Sboner, A., Iaquinta, P. J., Karthaus, W. R., Gopalan, A., Dowling, C., Wanjala, J. N., Undvall, E. A., Arora, V. K., Wongvipat, J., Kossai, M., Ramazanoglu, S., Barboza, L. P., Di, W., Cao, Z., Zhang, Q. F., Sirota, I., Ran, L., MacDonald, T. Y., Beltran, H., Mosquera, J. M., Touijer, K. A., Scardino, P. T., Laudone, V. P., Curtis, K. R., Rathkopf, D. E., Morris, M. J., Danila, D. C., Slovin, S. F., Solomon, S. B., Eastham, J. A., Chi, P., Carver, B., Rubin, M. A., Scher, H. I., Clevers, H., Sawyers, C. L. and Chen, Y. (2014). Organoid cultures derived from patients with advanced prostate cancer. Cell 159:176-187. PMID: 25201530. PMCID: 4237931.

Additional References for Example 1 labeled numerically.
1. Watson, P. A., Arora, V. K. & Sawyers, C. L. Emerging mechanisms of resistance to androgen receptor inhibitors in prostate cancer. Nat Rev Cancer 15, 701-711 (2015).
2. Beltran, H., Hruszkewycz, A., Scher, H. I., Hildesheim, J., Isaacs, J., Yu, E. Y., Kelly, K., Lin, D., Dicker, A., Arnold, J., Hecht, T., Wicha, M., Sears, R., Rowley, D., White, R., Gulley, J. L., Lee, J., Diaz Meco, M., Small, E. J., Shen, M., Knudsen, K., Goodrich, D. W., Lotan, T., Zoubeidi, A., Sawyers, C. L., Rudin, C. M., Loda, M., Thompson, T., Rubin, M. A., Tawab-Amiri, A., Dahut, W. & Nelson, P. S. The role of lineage plasticity in prostate cancer therapy resistance. Clin Cancer Res 25, 6916-6924 (2019).
3. Davies, A. H., Beltran, H. & Zoubeidi, A. Cellular plasticity and the neuroendocrine phenotype in prostate cancer. Nat Rev Urol 15, 271-286 (2018).
4. Rickman, D. S., Beltran, H., Demichelis, F. & Rubin, M. A. Biology and evolution of poorly differentiated neuroendocrine tumors. Nat Med 23, 1-10 (2017).
5. Rubin, M. A., Bristow, R. G., Thienger, P. D., Dive, C. & Imielinski, M. Impact of lineage plasticity to and from a neuroendocrine phenotype on progression and response in prostate and lung cancers. Mol Cell 80, 562-577 (2020).
6. Bennett, R. L., Swaroop, A., Troche, C. & Licht, J. D. The role of nuclear receptor-binding SET domain family histone lysine methyltransferases in cancer. Cold Spring Harb Perspect Med 7, a026708 (2017).
7. Zou, M., Toivanen, R., Mitrofanova, A., Floch, N., Hayati, S., Sun, Y., Le Magnen, C., Chester, D., Mostaghel, E. A., Califano, A., Rubin, M. A., Shen, M. M. & Abate-Shen, C. Transdifferentiation as a mechanism of treatment resistance in a mouse model of castration-resistant prostate cancer. Cancer Discov 7, 736-749 (2017).
8. Hanahan, D. Hallmarks of cancer: new dimensions. Cancer Discov 12, 31-46 (2022).
9. Beltran, H., Rickman, D. S., Park, K., Chae, S. S., Sboner, A., MacDonald, T. Y., Wang, Y., Sheikh, K. L., Terry, S., Tagawa, S. T., Dhir, R., Nelson, J. B., de la Taille, A., Allory, Y., Gerstein, M. B., Perner, S., Pienta, K. J., Chinnaiyan, A. M., Wang, Y., Collins, C. C., Gleave, M. E., Demichelis, F., Nanus, D. M. & Rubin, M. A. Molecular characterization of neuroendocrine prostate cancer and identification of new drug targets. Cancer Discov 1, 487-495 (2011).
10. Quigley, D. A., Dang, H. X., Zhao, S. G., Lloyd, P., Aggarwal, R., Alumkal, J. J., Foye, A., Kothari, V., Perry, M. D., Bailey, A. M., Playdle, D., Barnard, T. J., Zhang, L., Zhang, J., Youngren, J. F., Cieslik, M. P., Parolia, A., Beer, T. M., Thomas, G., Chi, K. N., Gleave, M., Lack, N. A., Zoubeidi, A., Reiter, R. E., Rettig, M. B., Witte, O., Ryan, C. J., Fong, L., Kim, W., Friedlander, T., Chou, J., Li, H., Das, R., Li, H., Moussavi-Baygi, R., Goodarzi, H., Gilbert, L. A., Lara, P. N., Jr., Evans, C. P., Goldstein, T. C., Stuart, J. M., Tomlins, S. A., Spratt, D. E., Cheetham, R. K., Cheng, D. T., Farh, K., Gehring, J. S., Hakenberg, J., Liao, A., Febbo, P. G., Shon, J., Sickler, B., Batzoglou, S., Knudsen, K. E., He, H. H., Huang, J., Wyatt, A. W., Dehm, S. M., Ashworth, A., Chinnaiyan, A. M., Maher, C. A., Small, E. J. & Feng, F. Y. Genomic hallmarks and structural variation in metastatic prostate cancer. Cell 174, 758-769 (2018).
11. Le Magnen, C., Shen, M. M. & Abate-Shen, C. Lineage plasticity in cancer progression and treatment. Annu Rev Cancer Biol 2, 271-289 (2018).
12. Labrecque, M. P., Coleman, I. M., Brown, L. G., True, L. D., Kollath, L., Lakely, B., Nguyen, H. M., Yang, Y. C., da Costa, R. M. G., Kaipainen, A., Coleman, R., Higano, C. S., Yu, E. Y., Cheng, H. H., Mostaghel, E. A., Montgomery, B., Schweizer, M. T., Hsieh, A. C., Lin, D. W., Corey, E., Nelson, P. S. & Morrissey, C. Molecular profiling stratifies diverse phenotypes of treatment-refractory metastatic castration-resistant prostate cancer. J Clin Invest 130, 4492-4505 (2019).
13. Bluemn, E. G., Coleman, I. M., Lucas, J. M., Coleman, R. T., Hernandez-Lopez, S., Tharakan, R., Bianchi-Frias, D., Dumpit, R. F., Kaipainen, A., Corella, A. N., Yang, Y. C., Nyquist, M. D., Mostaghel, E., Hsieh, A. C., Zhang, X., Corey, E., Brown, L. G., Nguyen, H. M., Pienta, K., Ittmann, M., Schweizer, M., True, L. D., Wise, D., Rennie, P. S., Vessella, R. L., Morrissey, C. & Nelson, P. S. Androgen receptor pathway-independent prostate cancer is sustained through FGF signaling. Cancer Cell 32, 474-489 (2017).
14. Su, W., Han, H. H., Wang, Y., Zhang, B., Zhou, B., Cheng, Y. K., Rumandia, A., Gurrapu, S., Chakraborty, G., Su, J., Yang, G., Liang, X., Wang, G., Rosen, N., Scher, H. I., Ouerfelli, O. & Giancotti, F. The Polycomb Repressor Complex 1 drives double negative prostate cancer metastasis by coordinating stemness and immune suppression. Cancer Cell 36, 139-155 (2019).
15. Zaffuto, E., Pompe, R., Zanaty, M., Bondarenko, H. D., Leyh-Bannurah, S. R., Moschini, M., Dell′Oglio, P., Gandaglia, G., Fossati, N., Stabile, A., Zorn, K. C., Montorsi, F., Briganti, A. & Karakiewicz, P. I. Contemporary incidence and cancer control outcomes of primary neuroendocrine prostate cancer: A SEER database analysis. Clin Genitourin Cancer 15, e793-e800 (2017).
16. Aggarwal, R., Huang, J., Alumkal, J. J., Zhang, L., Feng, F. Y., Thomas, G. V., Weinstein, A. S., Friedl, V., Zhang, C., Witte, O. N., Lloyd, P., Gleave, M., Evans, C. P., Youngren, J., Beer, T. M., Rettig, M., Wong, C. K., True, L., Foye, A., Playdle, D., Ryan, C. J., Lara, P., Chi, K. N., Uzunangelov, V., Sokolov, A., Newton, Y., Beltran, H., Demichelis, F., Rubin, M. A., Stuart, J. M. & Small, E. J. Clinical and genomic characterization of treatment-emergent small-cell neuroendocrine prostate cancer: a multi-institutional prospective study. J Clin Oncol 36, 2492-2503 (2018).
17. Abida, W., Cyrta, J., Heller, G., Prandi, D., Armenia, J., Coleman, I., Cieslik, M., Benelli, M., Robinson, D., Van Allen, E. M., Sboner, A., Fedrizzi, T., Mosquera, J. M., Robinson, B. D., De Sarkar, N., Kunju, L. P., Tomlins, S., Wu, Y. M., Nava Rodrigues, D., Loda, M., Gopalan, A., Reuter, V. E., Pritchard, C. C., Mateo, J., Bianchini, D., Miranda, S., Carreira, S., Rescigno, P., Filipenko, J., Vinson, J., Montgomery, R. B., Beltran, H., Heath, E. I., Scher, H. I., Kantoff, P. W., Taplin, M. E., Schultz, N., deBono, J. S., Demichelis, F., Nelson, P. S., Rubin, M. A., Chinnaiyan, A. M. & Sawyers, C. L. Genomic correlates of clinical outcome in advanced prostate cancer. Proc Natl Acad Sci USA 116, 11428-11436 (2019).
18. Laudato, S., Aparicio, A. & Giancotti, F. G. Clonal evolution and epithelial plasticity in the emergence of AR-independent prostate carcinoma. Trends Cancer 5, 440-455 (2019).
19. Tang, D. G. Understanding and targeting prostate cancer cell heterogeneity and plasticity. Semin Cancer Biol 82, 68-93 (2022).
20. Li, W. & Shen, M. M. Prostate cancer cell heterogeneity and plasticity: Insights from studies of genetically-engineered mouse models. Semin Cancer Biol 82, 60-67 (2022).
21. Quintanal-Villalonga, A., Chan, J. M., Yu, H. A., Pe′er, D., Sawyers, C. L., Sen, T. & Rudin, C. M. Lineage plasticity in cancer: a shared pathway of therapeutic resistance. Nat Rev Clin Oncol 17, 360-371 (2020).
22. Chan, J. M., Zaidi, S., Love, J. R., Zhao, J. L., Setty, M., Wadosky, K. M., Gopalan, A., Choo, Z. N., Persad, S., Choi, J., LaClair, J., Lawrence, K. E., Chaudhary, O., Xu, T., Masilionis, I., Linkov, I., Wang, S., Lee, C., Barlas, A., Morris, M. J., Mazutis, L., Chaligne, R., Chen, Y., Goodrich, D. W., Karthaus, W. R., Pe′er, D. & Sawyers, C. L. Lineage plasticity in prostate cancer depends on JAK/STAT inflammatory signaling. Science 377, 1180-1191 (2022).
23. Beltran, H., Prandi, D., Mosquera, J. M., Benelli, M., Puca, L., Cyrta, J., Marotz, C., Giannopoulou, E., Chakravarthi, B. V., Varambally, S., Tomlins, S. A., Nanus, D. M., Tagawa, S. T., Van Allen, E. M., Elemento, O., Sboner, A., Garraway, L. A., Rubin, M. A. & Demichelis, F. Divergent clonal evolution of castration-resistant neuroendocrine prostate cancer. Nat Med 22, 298-305 (2016).
24. Varambally, S., Dhanasekaran, S. M., Zhou, M., Barrette, T. R., Kumar-Sinha, C., Sanda, M. G., Ghosh, D., Pienta, K. J., Sewalt, R. G., Otte, A. P., Rubin, M. A. & Chinnaiyan, A. M. The polycomb group protein EZH2 is involved in progression of prostate cancer. Nature 419, 624-629 (2002).
25. Dardenne, E., Beltran, H., Benelli, M., Gayvert, K., Berger, A., Puca, L., Cyrta, J., Sboner, A., Noorzad, Z., MacDonald, T., Cheung, C., Yuen, K. S., Gao, D., Chen, Y., Eilers, M., Mosquera, J. M., Robinson, B. D., Elemento, O., Rubin, M. A., Demichelis, F. & Rickman, D. S. N-Myc induces an EZH2-mediated transcriptional program driving neuroendocrine prostate cancer. Cancer Cell 30, 563-577 (2016).
26. Ku, S. Y., Rosario, S., Wang, Y., Mu, P., Seshadri, M., Goodrich, Z. W., Goodrich, M. M., Labbe, D. P., Gomez, E. C., Wang, J., Long, H. W., Xu, B., Brown, M., Loda, M., Sawyers, C. L., Ellis, L. & Goodrich, D. W. Rb1 and Trp53 cooperate to suppress prostate cancer lineage plasticity, metastasis, and antiandrogen resistance. Science 355, 78-83 (2017).
27. Puca, L., Bareja, R., Prandi, D., Shaw, R., Benelli, M., Karthaus, W. R., Hess, J., Sigouros, M., Donoghue, A., Kossai, M., Gao, D., Cyrta, J., Sailer, V., Vosoughi, A., Pauli, C., Churakova, Y., Cheung, C., Deonarine, L. D., McNary, T. J., Rosati, R., Tagawa, S. T., Nanus, D. M., Mosquera, J. M., Sawyers, C. L., Chen, Y., Inghirami, G., Rao, R. A., Grandori, C., Elemento, O., Sboner, A., Demichelis, F., Rubin, M. A. & Beltran, H. Patient derived organoids to model rare prostate cancer phenotypes. Nat Commun 9, 2404 (2018).
28. Li, Y., Trojer, P., Xu, C. F., Cheung, P., Kuo, A., Drury, W. J., 3^{rd}, Qiao, Q., Neubert, T. A., Xu, R. M., Gozani, O. & Reinberg, D. The target of the NSD family of histone lysine methyltransferases depends on the nature of the substrate. J Biol Chem 284, 34283-34295 (2009).
29. Kuo, A. J., Cheung, P., Chen, K., Zee, B. M., Kioi, M., Lauring, J., Xi, Y., Park, B. H., Shi, X., Garcia, B. A., Li, W. & Gozani, O. NSD2 links 47emethylation of histone H3 at lysine 36 to oncogenic programming. Mol Cell 44, 609-620 (2011).
30. Martinez-Garcia, E., Popovic, R., Min, D. J., Sweet, S. M., Thomas, P. M., Zamdborg, L., Heffner, A., Will, C., Lamy, L., Staudt, L. M., Levens, D. L., Kelleher, N. L. & Licht, J. D. The MMSET histone methyl transferase switches global histone methylation and alters gene expression in t(4; 14) multiple myeloma cells. Blood 117, 211-220 (2011).
31. Popovic, R., Martinez-Garcia, E., Giannopoulou, E. G., Zhang, Q., Zhang, Q., Ezponda, T., Shah, M. Y., Zheng, Y., Will, C. M., Small, E. C., Hua, Y., Bulic, M., Jiang, Y., Carrara, M., Calogero, R. A., Kath, W. L., Kelleher, N. L., Wang, J. P., Elemento, O. & Licht, J. D. Histone methyltransferase MMSET/NSD2 alters EZH2 binding and reprograms the myeloma epigenome through global and focal changes in H3K36 and H3K27 methylation. PloS Genet 10, e1004566 (2014).
32. Streubel, G., Watson, A., Jammula, S. G., Scelfo, A., Fitzpatrick, D. J., Oliviero, G., McCole, R., Conway, E., Glancy, E., Negri, G. L., Dillon, E., Wynne, K., Pasini, D., Krogan, N. J., Bracken, A. P. & Cagney, G. The H3K36me2 methyltransferase Nsd1 demarcates PRC2-mediated H3K27me2 and H3K27me3 domains in embryonic stem cells. Mol Cell 70, 371-379 e375 (2018).
33. Yuan, W., Xu, M., Huang, C., Liu, N., Chen, S. & Zhu, B. H3K36 methylation antagonizes PRC2-mediated H3K27 methylation. J Biol Chem 286, 7983-7989 (2011).
34. Schmitges, F. W., Prusty, A. B., Faty, M., Stutzer, A., Lingaraju, G. M., Aiwazian, J., Sack, R., Hess, D., Li, L., Zhou, S., Bunker, R. D., Wirth, U., Bouwmeester, T., Bauer, A., Ly-Hartig, N., Zhao, K., Chan, H., Gu, J., Gut, H., Fischle, W., Muller, J. & Thoma, N. H. Histone methylation by PRC2 is inhibited by active chromatin marks. Mol Cell 42, 330-341 (2011).
35. Weinberg, D. N., Papillon-Cavanagh, S., Chen, H., Yue, Y., Chen, X., Rajagopalan, K. N., Horth, C., McGuire, J. T., Xu, X., Nikbakht, H., Lemiesz, A. E., Marchione, D. M., Marunde, M. R., Meiners, M. J., Cheek, M. A., Keogh, M. C., Bareke, E., Djedid, A., Harutyunyan, A. S., Jabado, N., Garcia, B. A., Li, H., Allis, C. D., Majewski, J. & Lu, C. The histone mark H3K36me2 recruits DNMT3A and shapes the intergenic DNA methylation landscape. Nature 573, 281-286 (2019).
36. Shirane, K., Miura, F., Ito, T. & Lorincz, M. C. NSD1-deposited H3K36me2 directs de novo methylation in the mouse male germline and counteracts Polycomb-associated silencing. Nat Genet 52, 1088-1098 (2020).
37. Hamagami, N., Wu, D. Y., Clemens, A. W., Nettles, S. A., Li, A. & Gabel, H. W. NSD1 deposits histone H3 lysine 36 dimethylation to pattern non-CG DNA methylation in neurons. Mol Cell 83, 1412-1428 (2023).
38. Wang, X., Kruithof-de Julio, M., Economides, K. D., Walker, D., Yu, H. L., Halili, M. V., Hu, Y. P., Price, S. M., Abate-Shen, C. & Shen, M. M. A luminal epithelial stem cell that is a cell of origin for prostate cancer. Nature 461, 495-500 (2009).
39. Alvarez, M. J., Shen, Y., Giorgi, F. M., Lachmann, A., Ding, B. B., Ye, B. H. & Califano, A. Functional characterization of somatic mutations in cancer using network-based inference of protein activity. Nat Genet 48, 838-847 (2016).
40. Margolin, A. A., Nemenman, I., Basso, K., Wiggins, C., Stolovitzky, G., Dalla Favera, R. & Califano, A. ARACNE: an algorithm for the reconstruction of gene regulatory networks in a mammalian cellular context. BMC Bioinformatics 7 Suppl 1, S7 (2006).
41. Lachmann, A., Giorgi, F. M., Lopez, G. & Califano, A. ARACNe-AP: gene network reverse engineering through adaptive partitioning inference of mutual information. Bioinformatics 32, 2233-2235 (2016).
42. Gulati, G. S., Sikandar, S. S., Wesche, D. J., Manjunath, A., Bharadwaj, A., Berger, M. J., Ilagan, F., Kuo, A. H., Hsieh, R. W., Cai, S., Zabala, M., Scheeren, F. A., Lobo, N. A., Qian, D., Yu, F. B., Dirbas, F. M., Clarke, M. F. & Newman, A. M. Single-cell transcriptional diversity is a hallmark of developmental potential. Science 367, 405-411 (2020).
43. Han, H., Wang, Y., Curto, J., Gurrapu, S., Laudato, S., Rumandla, A., Chakraborty, G., Wang, X., Chen, H., Jiang, Y., Kumar, D., Caggiano, E. G., Capogiri, M., Zhang, B., Ji, Y., Maity, S. N., Hu, M., Bai, S., Aparicio, A. M., Efstathiou, E., Logothetis, C. J., Navin, N., Navone, N. M., Chen, Y. & Giancotti, F. G. Mesenchymal and stem-like prostate cancer linked to therapy-induced lineage plasticity and metastasis. Cell Rep 39, 110595 (2022).
44. Kaya-Okur, H. S., Wu, S. J., Codomo, C. A., Pledger, E. S., Bryson, T. D., Henikoff, J. G., Ahmad, K. & Henikoff, S. CUT & Tag for efficient epigenomic profiling of small samples and single cells. Nat Commun 10, 1930 (2019).
45. Sun, Z., Lin, Y., Islam, M. T., Koche, R., Hedehus, L., Liu, D., Huang, C., Vierbuchen, T., Sawyers, C. L. & Helin, K. Chromatin regulation of transcriptional enhancers and cell fate by the Sotos syndrome gene NSD1. Mol Cell, doi: 10.1016/j.molcel.2023.1006.1007 (2023).
46. Aytes, A., Giacobbe, A., Mitrofanova, A., Ruggero, K., Cyrta, J., Arriaga, J., Palomero, L., Farran-Matas, S., Rubin, M. A., Shen, M. M., Califano, A. & Abate-Shen, C. NSD2 is a conserved driver of metastatic prostate cancer progression. Nat Commun 9, 5201 (2018).
47. Yang, P., Guo, L., Duan, Z. J., Tepper, C. G., Xue, L., Chen, X., Kung, H. J., Gao, A. C., Zou, J. X. & Chen, H. W. Histone methyltransferase NSD2/MMSET mediates constitutive NF-kappaB signaling for cancer cell proliferation, survival, and tumor growth via a feed-forward loop. Mol Cell Biol 32, 3121-3131 (2012).
48. Filon, M., Gawdzik, J., Truong, A., Allen, G., Huang, W., Khemees, T., Machhi, R., Lewis, P., Yang, B., Denu, J. & Jarrard, D. Tandem histone methyltransferase upregulation defines a unique aggressive prostate cancer phenotype. Br J Cancer 125, 247-254 (2021).
49. Lu, C., Jain, S. U., Hoelper, D., Bechet, D., Molden, R. C., Ran, L., Murphy, D., Venneti, S., Hameed, M., Pawel, B. R., Wunder, J. S., Dickson, B. C., Lundgren, S. M., Jani, K. S., De Jay, N., Papillon-Cavanagh, S., Andrulis, I. L., Sawyer, S. L., Grynspan, D., Turcotte, R. E., Nadaf, J., Fahiminiyah, S., Muir, T. W., Majewski, J., Thompson, C. B., Chi, P., Garcia, B. A., Allis, C. D., Jabado, N. & Lewis, P. W. Histone H3K36 mutations promote sarcomagenesis through altered histone methylation landscape. Science 352, 844-849 (2016).
50. Fang, D., Gan, H., Lee, J. H., Han, J., Wang, Z., Riester, S. M., Jin, L., Chen, J., Zhou, H., Wang, J., Zhang, H., Yang, N., Bradley, E. W., Ho, T. H., Rubin, B. P., Bridge, J. A., Thibodeau, S. N., Ordog, T., Chen, Y., van Wijnen, A. J., Oliveira, A. M., Xu, R. M., Westendorf, J. J. & Zhang, Z. The histone H3.3K36M mutation reprograms the epigenome of chondroblastomas. Science 352, 1344-1348 (2016).
51. Rajagopalan, K. N., Chen, X., Weinberg, D. N., Chen, H., Majewski, J., Allis, C. D. & Lu, C. Depletion of H3K36me2 recapitulates epigenomic and phenotypic changes induced by the H3.3K36M oncohistone mutation. Proc Natl Acad Sci USA 118, e2021795118 (2021).
52. Tang, F., Xu, D., Wang, S., Wong, C. K., Martinez-Fundichely, A., Lee, C. J., Cohen, S., Park, J., Hill, C. E., Eng, K., Bareja, R., Han, T., Liu, E. M., Palladino, A., Di, W., Gao, D., Abida, W., Beg, S., Puca, L., Meneses, M., de Stanchina, E., Berger, M. F., Gopalan, A., Dow, L. E., Mosquera, J. M., Beltran, H., Sternberg, C. N., Chi, P., Scher, H. I., Sboner, A., Chen, Y. & Khurana, E. Chromatin profiles classify castration-resistant prostate cancers suggesting therapeutic targets. Science 376, eabe1505 (2022).
53. Pomerantz, M. M., Li, F., Takeda, D. Y., Lenci, R., Chonkar, A., Chabot, M., Cejas, P., Vazquez, F., Cook, J., Shivdasani, R. A., Bowden, M., Lis, R., Hahn, W. C., Kantoff, P. W., Brown, M., Loda, M., Long, H. W. & Freedman, M. L. The androgen receptor cistrome is extensively reprogrammed in human prostate tumorigenesis. Nat Genet 47, 1346-1351 (2015).
54. Pomerantz, M. M., Qiu, X., Zhu, Y., Takeda, D. Y., Pan, W., Baca, S. C., Gusev, A., Korthauer, K. D., Severson, T. M., Ha, G., Viswanathan, S. R., Seo, J. H., Nguyen, H. M., Zhang, B., Pasaniuc, B., Giambartolomei, C., Alaiwi, S. A., Bell, C. A., O'Connor, E. P., Chabot, M. S., Stillman, D. R., Lis, R., Font-Tello, A., Li, L., Cejas, P., Bergman, A. M., Sanders, J., van der Poel, H. G., Gayther, S. A., Lawrenson, K., Fonseca, M. A. S., Reddy, J., Corona, R. I., Martovetsky, G., Egan, B., Choueiri, T., Ellis, L., Garraway, I. P., Lee, G. M., Corey, E., Long, H. W., Zwart, W. & Freedman, M. L. Prostate cancer reactivates developmental epigenomic programs during metastatic progression. Nat Genet 52, 790-799 (2020).
55. Severson, T., Qiu, X., Alshalalfa, M., Sjostrom, M., Quigley, D., Bergman, A., Long, H., Feng, F., Freedman, M. L., Zwart, W. & Pomerantz, M. M. Androgen receptor reprogramming demarcates prognostic, context-dependent gene sets in primary and metastatic prostate cancer. Clin Epigenetics 14, 60 (2022).
56. Xiao, L., Parolia, A., Qiao, Y., Bawa, P., Eyunni, S., Mannan, R., Carson, S. E., Chang, Y., Wang, X., Zhang, Y., Vo, J. N., Kregel, S., Simko, S. A., Delekta, A. D., Jaber, M., Zheng, H., Apel, I. J., McMurry, L., Su, F., Wang, R., Zelenka-Wang, S., Sasmal, S., Khare, L., Mukherjee, S., Abbineni, C., Aithal, K., Bhakta, M. S., Ghurye, J., Cao, X., Navone, N. M., Nesvizhskii, A. I., Mehra, R., Vaishampayan, U., Blanchette, M., Wang, Y., Samajdar, S., Ramachandra, M. & Chinnaiyan, A. M. Targeting SWI/SNF ATPases in enhancer-addicted prostate cancer. Nature 601, 434-439 (2022).
57. Wang, Q., Li, W., Zhang, Y., Yuan, X., Xu, K., Yu, J., Chen, Z., Beroukhim, R., Wang, H., Lupien, M., Wu, T., Regan, M. M., Meyer, C. A., Carroll, J. S., Manrai, A. K., Janne, O. A., Balk, S. P., Mehra, R., Han, B., Chinnaiyan, A. M., Rubin, M. A., True, L., Fiorentino, M., Fiore, C., Loda, M., Kantoff, P. W., Liu, X. S. & Brown, M. Androgen receptor regulates a distinct transcription program in androgen-independent prostate cancer. Cell 138, 245-256 (2009).
58. Han, M., Li, F., Zhang, Y., Dai, P., He, J., Li, Y., Zhu, Y., Zheng, J., Huang, H., Bai, F. & Gao, D. FOXA2 drives lineage plasticity and KIT pathway activation in neuroendocrine prostate cancer. Cancer Cell 40, 1306-1323 (2022).
59. Rotinen, M., You, S., Yang, J., Coetzee, S. G., Reis-Sobreiro, M., Huang, W. C., Huang, F., Pan, X., Yanez, A., Hazelett, D. J., Chu, C. Y., Steadman, K., Morrissey, C. M., Nelson, P. S., Corey, E., Chung, L. W. K., Freedland, S. J., Di Vizio, D., Garraway, I. P., Murali, R., Knudsen, B. S. & Freeman, M. R. ONECUT2 is a targetable master regulator of lethal prostate cancer that suppresses the androgen axis. Nat Med 24, 1887-1898 (2018).
60. Kang, H. B., Choi, Y., Lee, J. M., Choi, K. C., Kim, H. C., Yoo, J. Y., Lee, Y. H. & Yoon, H. G. The histone methyltransferase, NSD2, enhances androgen receptor-mediated transcription. FEBS Lett 583, 1880-1886 (2009).
61. Li, N., Xue, W., Yuan, H., Dong, B., Ding, Y., Liu, Y., Jiang, M., Kan, S., Sun, T., Ren, J., Pan, Q., Li, X., Zhang, P., Hu, G., Wang, Y., Wang, X., Li, Q. & Qin, J. AKT-mediated stabilization of histone methyltransferase WHSC1 promotes prostate cancer metastasis. J Clin Invest 127, 1284-1302 (2017).
62. Ezponda, T., Popovic, R., Shah, M. Y., Martinez-Garcia, E., Zheng, Y., Min, D. J., Will, C., Neri, A., Kelleher, N. L., Yu, J. & Licht, J. D. The histone methyltransferase MMSET/WHSC1 activates TWIST1 to promote an epithelial-mesenchymal transition and invasive properties of prostate cancer. Oncogene 32, 2882-2890 (2013).
63. Yuan, S., Natesan, R., Sanchez-Rivera, F. J., Li, J., Bhanu, N. V., Yamazoe, T., Lin, J. H., Merrell, A. J., Sela, Y., Thomas, S. K., Jiang, Y., Plesset, J. B., Miller, E. M., Shi, J., Garcia, B. A., Lowe, S. W., Asangani, I. A. & Stanger, B. Z. Global regulation of the histone mark H3K36me2 underlies epithelial plasticity and metastatic progression. Cancer Discov 10, 854-871 (2020).
64. Chesi, M., Nardini, E., Lim, R. S., Smith, K. D., Kuehl, W. M. & Bergsagel, P. L. The t(4; 14) translocation in myeloma dysregulates both FGFR3 and a novel gene, MMSET, resulting in IgH/MMSET hybrid transcripts. Blood 92, 3025-3034 (1998).
65. Jaffe, J. D., Wang, Y., Chan, H. M., Zhang, J., Huether, R., Kryukov, G. V., Bhang, H. E., Taylor, J. E., Hu, M., Englund, N. P., Yan, F., Wang, Z., Robert McDonald, E., 3rd, Wei, L., Ma, J., Easton, J., Yu, Z., deBeaumount, R., Gibaja, V., Venkatesan, K., Schlegel, R., Sellers, W. R., Keen, N., Liu, J., Caponigro, G., Barretina, J., Cooke, V. G., Mullighan, C., Carr, S. A., Downing, J. R., Garraway, L. A. & Stegmeier, F. Global chromatin profiling reveals NSD2 mutations in pediatric acute lymphoblastic leukemia. Nat Genet 45, 1386-1391 (2013).
66. Papillon-Cavanagh, S., Lu, C., Gayden, T., Mikael, L. G., Bechet, D., Karamboulas, C., Ailles, L., Karamchandani, J., Marchione, D. M., Garcia, B. A., Weinreb, I., Goldstein, D., Lewis, P. W., Dancu, O. M., Dhaliwal, S., Stecho, W., Howlett, C. J., Mymryk, J. S., Barrett, J. W., Nichols, A. C., Allis, C. D., Majewski, J. & Jabado, N. Impaired H3K36 methylation defines a subset of head and neck squamous cell carcinomas. Nat Genet 49, 180-185 (2017).
67. Yuan, G., Flores, N. M., Hausmann, S., Lofgren, S. M., Kharchenko, V., Angulo-Ibanez, M., Sengupta, D., Lu, X., Czaban, I., Azhibek, D., Vicent, S., Fischle, W., Jaremko, M., Fang, B., Wistuba, II, Chua, K. F., Roth, J. A., Minna, J. D., Shao, N. Y., Jaremko, L., Mazur, P. K. & Gozani, O. Elevated NSD3 histone methylation activity drives squamous cell lung cancer. Nature 590, 504-508 (2021).
68. Sengupta, D., Zeng, L., Li, Y., Hausmann, S., Ghosh, D., Yuan, G., Nguyen, T. N., Lyu, R., Caporicci, M., Morales Benitez, A., Coles, G. L., Kharchenko, V., Czaban, I., Azhibek, D., Fischle, W., Jaremko, M., Wistuba, II, Sage, J., Jaremko, L., Li, W., Mazur, P. K. & Gozani, O. NSD2 dimethylation at H3K36 promotes lung adenocarcinoma pathogenesis. Mol Cell 81, 4481-4492 e4489 (2021).
69. Wang, Z. A., Mitrofanova, A., Bergren, S. K., Abate-Shen, C., Cardiff, R. D., Califano, A. & Shen, M. M. Lineage analysis of basal epithelial cells reveals their unexpected plasticity and supports a cell-of-origin model for prostate cancer heterogeneity. Nat Cell Biol 15, 274-283 (2013).
70. Chua, C. W., Shibata, M., Lei, M., Toivanen, R., Barlow, L. J., Bergren, S. K., Badani, K. K., McKiernan, J. M., Benson, M. C., Hibshoosh, H. & Shen, M. M. Single luminal epithelial progenitors can generate prostate organoids in culture. Nat Cell Biol 16, 951-961 (2014).
71. Shechter, D., Dormann, H. L., Allis, C. D. & Hake, S. B. Extraction, purification and analysis of histones. Nat Protoc 2, 1445-1457 (2007).
72. Wolf, F. A., Angerer, P. & Theis, F. J. SCANPY: large-scale single-cell gene expression data analysis. Genome Biol 19, 15 (2018).
73. Haghverdi, L., Buettner, F. & Theis, F. J. Diffusion maps for high-dimensional single-cell analysis of differentiation data. Bioinformatics 31, 2989-2998 (2015).
74. Stuart, T., Srivastava, A., Madad, S., Lareau, C. A. & Satija, R. Single-cell chromatin state analysis with Signac. Nat Methods 18, 1333-1341 (2021).

### Example 2—Derivation and Maintenance of the Mouse Neuroendocrine Organoids of Example

Tumor tissues from NPp53 mice were cut into two pieces. Half of the tissue was reserved in 10% formalin for fixation prior to paraffin embedding and immunostaining. The other half was minced in 0.2% collagenase IV (Thermo Fisher Scientific, 17104019) with scissors. Diced tumor tissues were incubated in 0.2% Collagenase IV at 37° C. for 30 min and then neutralized with 1:10 Hank's buffer (STEMCELL Technologies, 37150) supplemented with 5% charcoal-stripped Fetal Bovine Serum (CS-FBS) (Gemini, 100-119) and 10 μM Rock inhibitor (STEMCELL Technologies, Y-27632). After spinning down at 1000 rpm for 10 min, the pellets were incubated with prewarmed TrypLE (Thermo Fisher Scientific, 12605010) at 37° C. for 10 min. The cell suspension was then neutralized 1:10 with PBS, passed through a 100 μM sterile cell strainer (Corning, 352360) and spun down at 1000 rpm for 10 min. Prior to plating, cell number was counted in a TC20 automated cell counter (Bio-Rad, 1450102). Cells were resuspended in primary NE organoid culture media supplemented with 10 μM Rock inhibitor, 10 μM A83-01 (Tocris, 2939) and 5% Matrigel (Corning, 354234) and seeded at ~50,000 cells per well in 96-well low attachment plates (Corning, 3474). The primary NE organoid culture medium was composed of hepatocyte culture medium (Corning, 355056), 5% CS-FBS, 1× GlutaMAX™ Supplement (Thermo Fisher Scientific, 35050061), 5 ng/ml EGF, 100 μg/ml Primocin (Invivogen, ant-pm-1) and 100 nM dihydrotestosterone (DHT).

NE organoids were maintained in two ways. For heterogenous NE organoids, such as NPPO-1 and NPPO-5, primary NE organoid culture medium was applied and replenished every 4 days. For homogeneous NE organoids, such as NPPO-1NE, NPPO-2, NPPO-4 and NPPO-6, we used NE organoid culture medium that was identical to the primary medium except that EGF was completely removed.

### Example 3—Disruption of NSD2 Signaling

A Small Molecule that Targets Nsd2

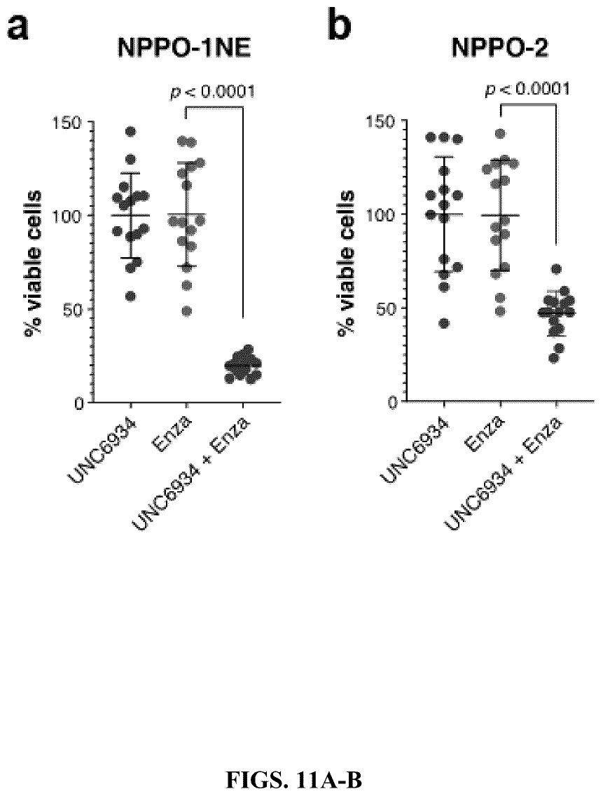

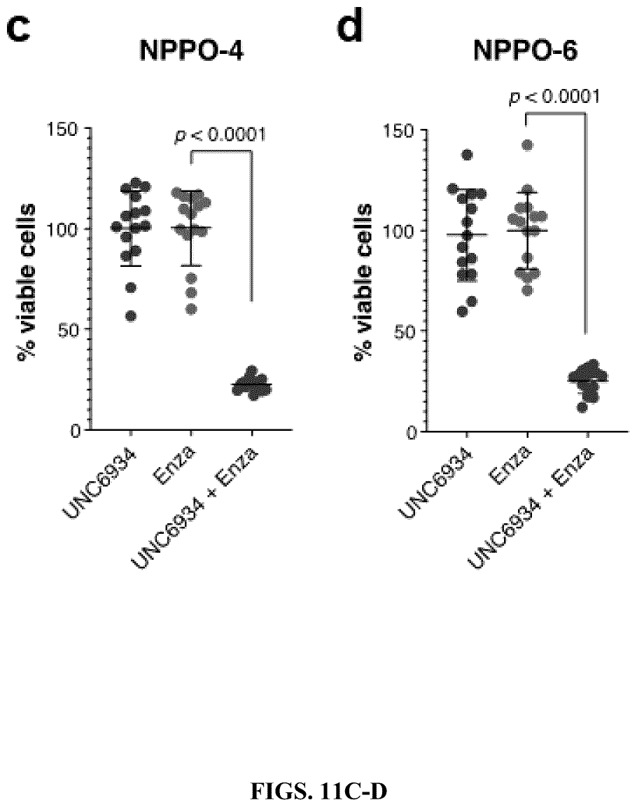

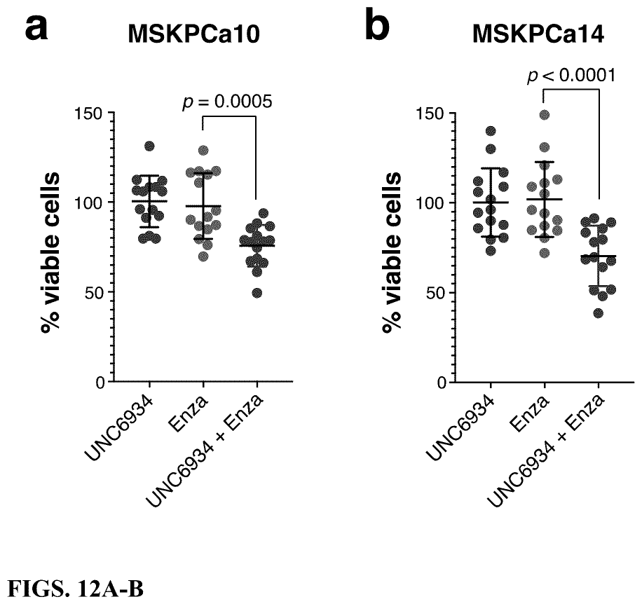

Recent work has identified a small molecule known as UNC6934 that can effectively bind to the PWWP1 domain of NSD2, but lacks the ability to inhibit catalytic function of NSD2, and thus does not affect H3K36 dimethylation [3]. We obtained this molecule from a commercial vendor (MedChemExpress) and tested whether it could inhibit neuroendocrine organoid growth in combination with enzalutamide. We found that UNC6934 by itself had no effect on the phenotype or growth of NPPO organoids, but in combination with enzalutamide had a strong growth inhibitory effect (FIGS. 11A-D). We obtained similar results with two independent human NEPC organoid lines, MSKPCa10 and MSKPCa14 (FIG. 12A-B); however, we did not observe any response in a third NEPC line, WCM154, suggesting heterogeneity of response in human NEPC. These findings indicate that UNC6934 can reverse the castration-resistance of mouse NPPO organoids and at least some human NEPC organoids, thereby restoring response to androgen receptor inhibitors.

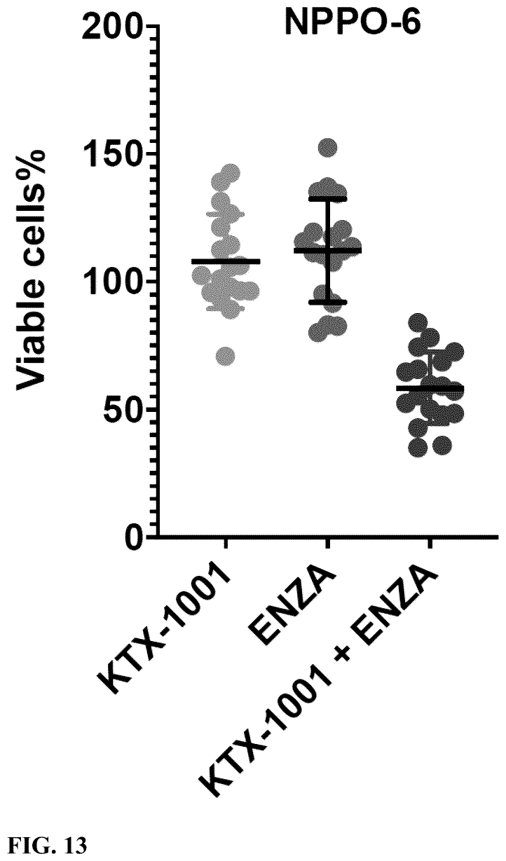

Similar to UNC6934, KTX-1001 by itself had no effect on the growth of NPPO-6 organoids. However, KTX-1001 in combination with enzalutamide had a strong growth inhibitory effect (FIG. 13). These results suggest that KTX-1001 has a synergistic effect with enzalutamide in inhibiting growth of the mouse neuroendocrine prostate cancer. KTX-1001 was obtained from a commercial vendor (Excenen, catalog number EX-A5782).

### Example 4—Pharmacological Inhibition of NSD2 and AR Suppresses Growth and Promotes Apoptosis of Human CRPC Organoids and Xenografts

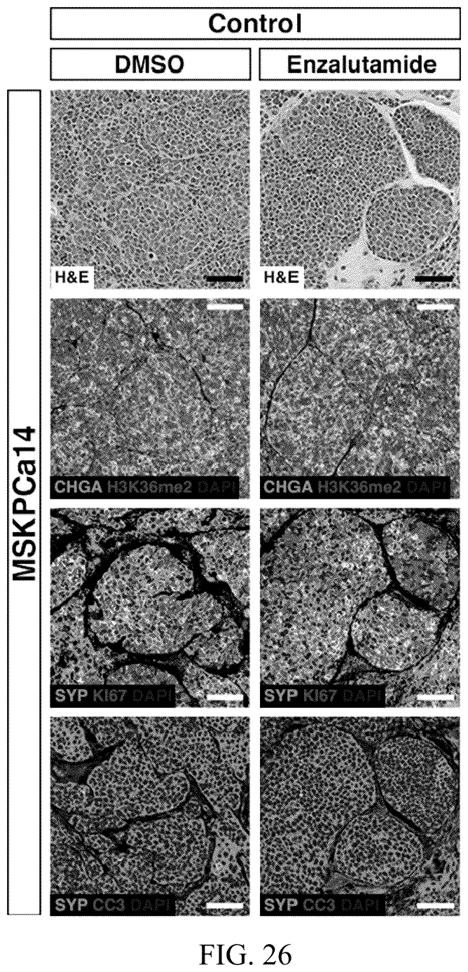

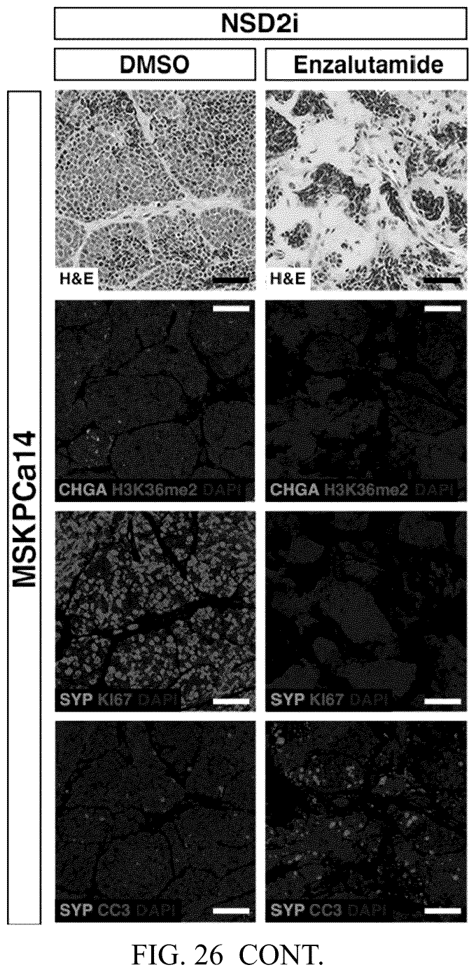

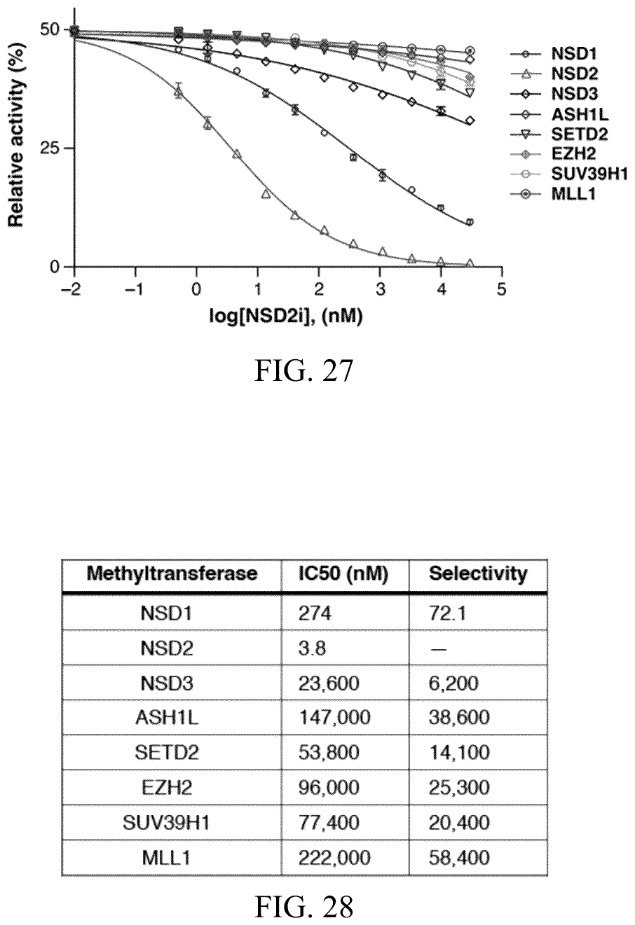

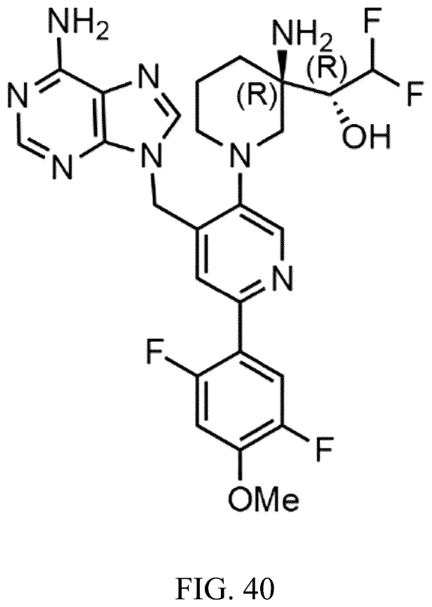

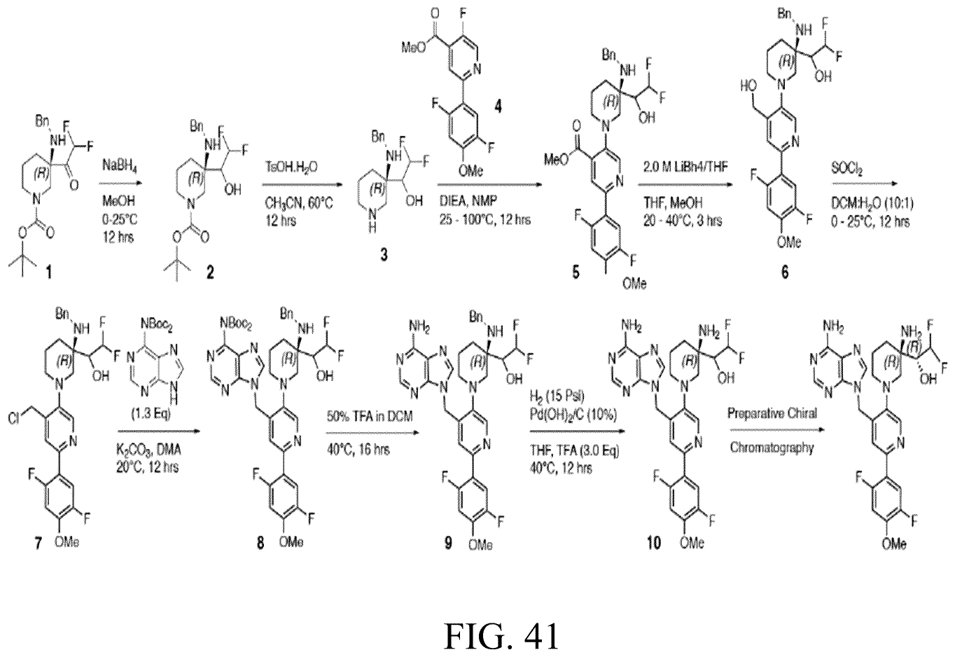

We investigated whether pharmacological inhibition of NSD2 could reverse lineage plasticity and restore enzalutamide sensitivity in organoids from CRPC-NE as well as other CRPC subtypes. We synthesized a catalytic NSD2 inhibitor, “NSD2iA” (FIGS. 40 and 41), that is similar to the active component of KTX-1001, a catalytic NSD2 inhibitor that is currently being tested in an early-phase clinical trial for t(4; 14) translocation-positive multiple myeloma (NCT05651932). We found that this compound (hereafter, “NSD2iA”) was highly specific for inhibiting NSD2 in vitro methylation activity on nucleosomes with a IC₅₀ of 3.8 nM, and displayed greater than 10,000-fold selectivity against other methyltransferases tested, except for NSD1 which had an IC₅₀ of 274 nM (FIGS. 27 and 28).

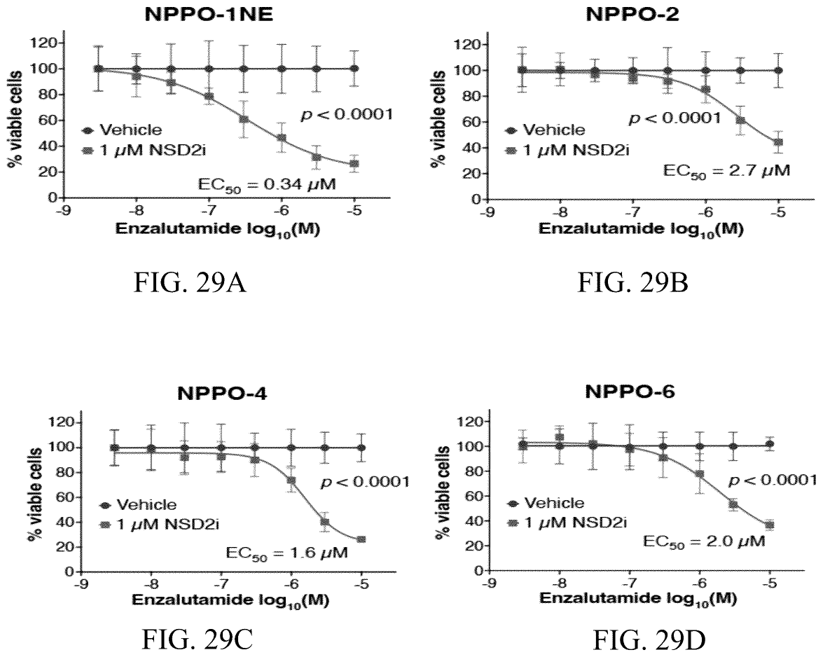

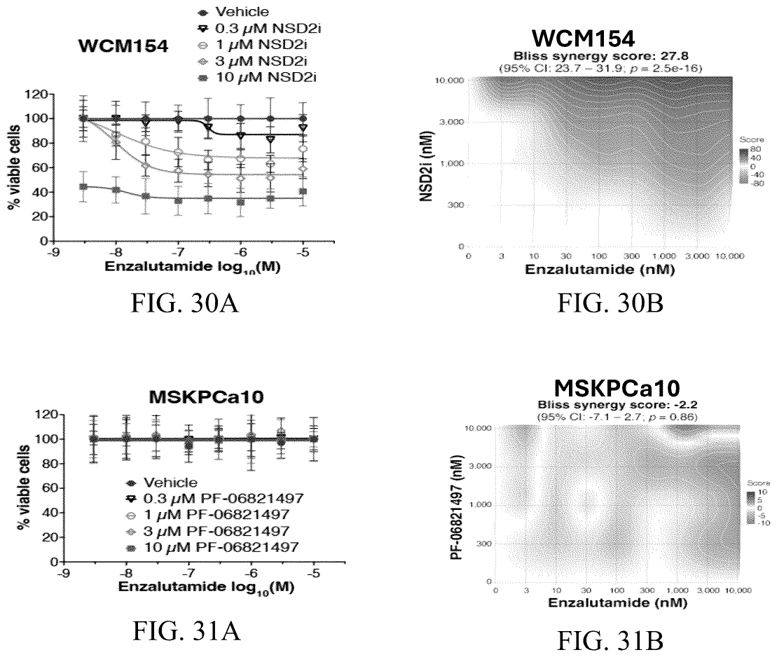

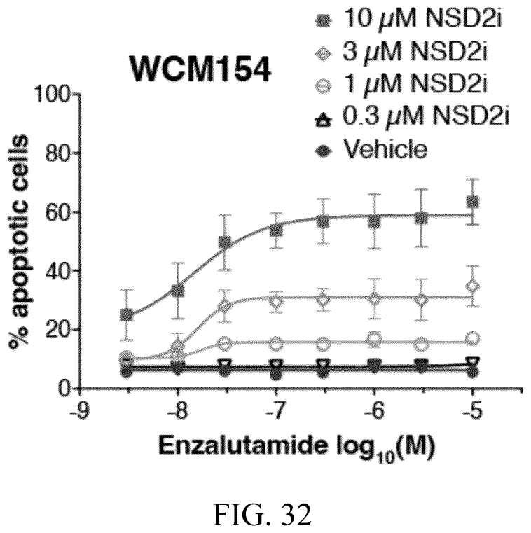

We treated mouse organoids with NSD2iA for 12 days prior to assaying for enzalutamide response. We found that NSD2iA treatment of the NPPO-1NE, NPPO-2, NPPO-4, and NPPO-6 organoid lines increased sensitivity to enzalutamide, achieving IC₅₀ values similar to those observed following NSD2 KO or H3.3K36M expression (FIGS. 29A-D). This shows that NSD2iA has the ability to confer enzalutamide sensitivity to mouse CRPC-NE organoids.

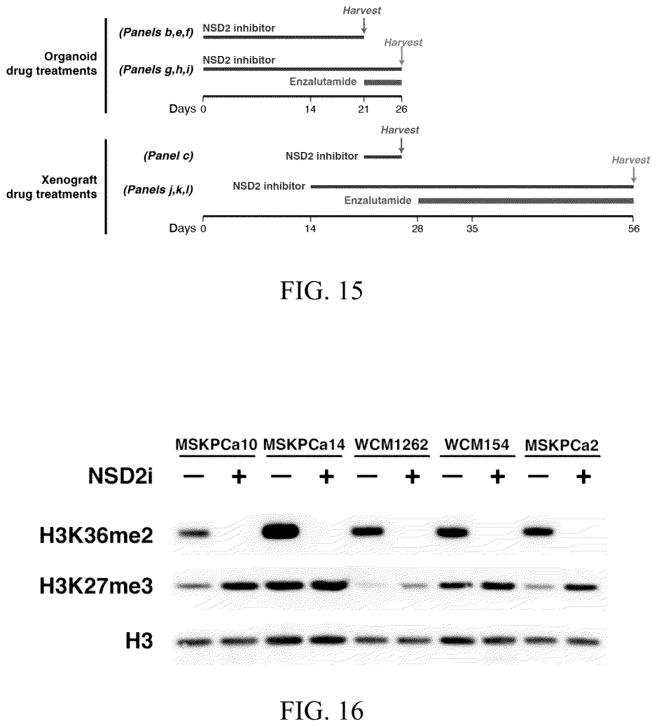

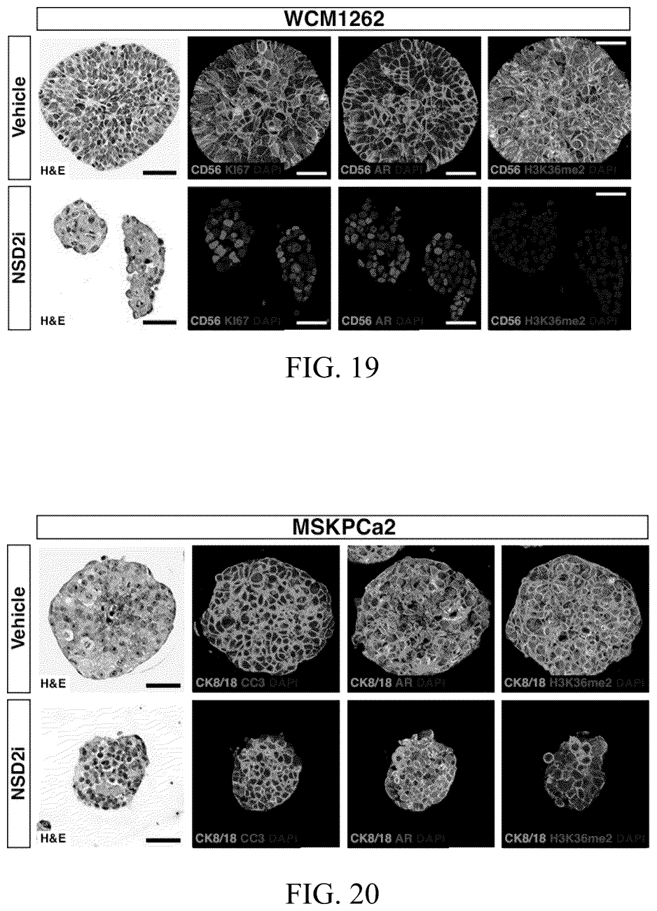

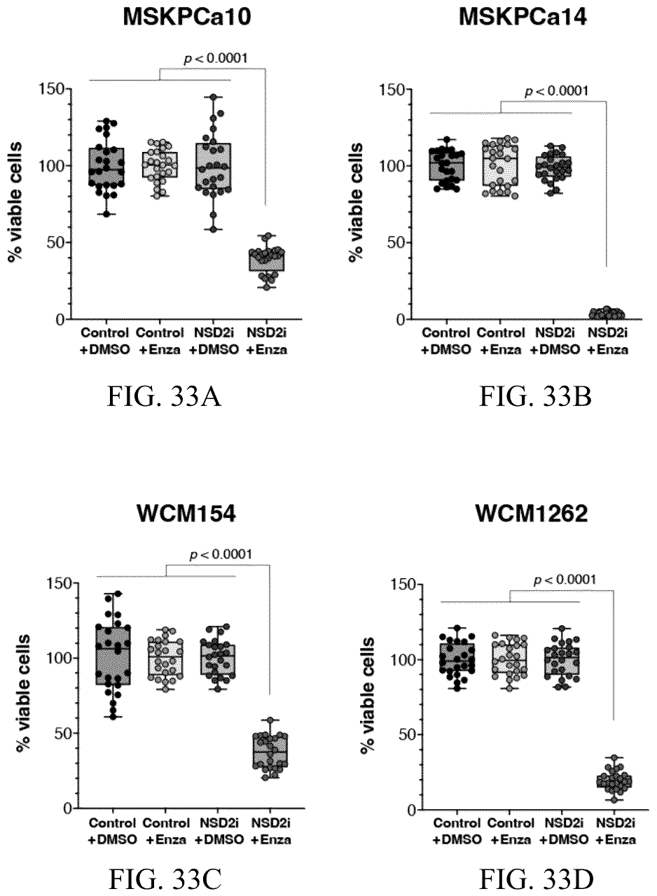

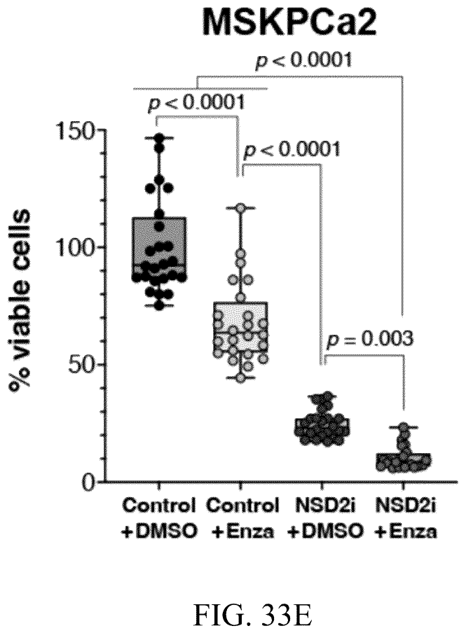

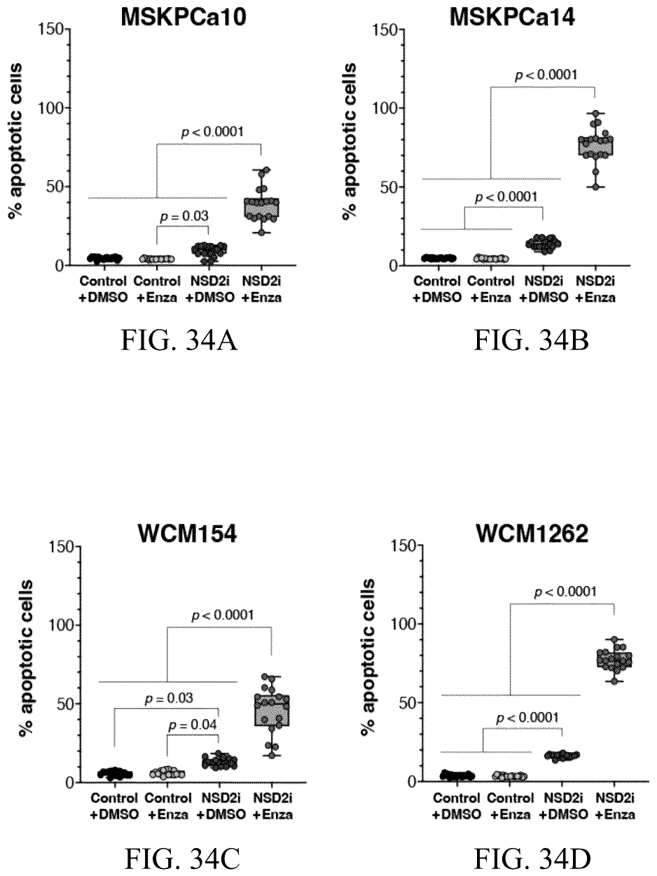

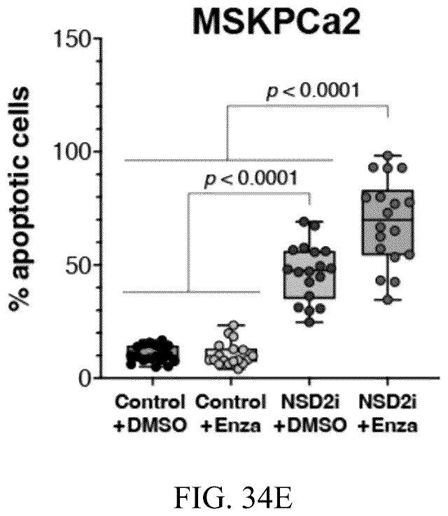

We tested human CRPC organoids lines for their response to NSD2iA, using five independent organoid lines that differ in their mutational status and subtype classifications (FIG. 18). See also Tang et al. (2022). The MSKPCa10, MSKPCa14, and WCM154 lines have been subtyped as CRPC-NE. The WCM1262 line is the CRPC-WNT subtype, and MSKPCa2 represents the CRPC-AR subtype. After treatment with NSD2iA for 3 weeks (FIG. 15), all five organoid lines displayed dramatic loss of H3K36me2 marks as well as modest up-regulation of H3K27me3 marks as detected by Western blotting (FIG. 16); however, treatment with NSD2iA had no effects on cell viability, except for MSKPCa2 (FIGS. 33A-E). Furthermore, immunostaining of NSD2iA-treated organoids revealed loss of H3K36me2 in both WCM1262 and MSKPCa2 as well as loss of the lineage marker CD56 and up-regulation of AR expression in WCM1262 (FIGS. 19 and 20). Interestingly, treatment of MSKPCa2 organoids with NSD2iA resulted in reduction of organoid size and substantial immunostaining for cleaved caspase 3 (CC3), indicating extensive apoptosis (FIG. 20).

### Synergism of NSD2iA and Enzalutamide

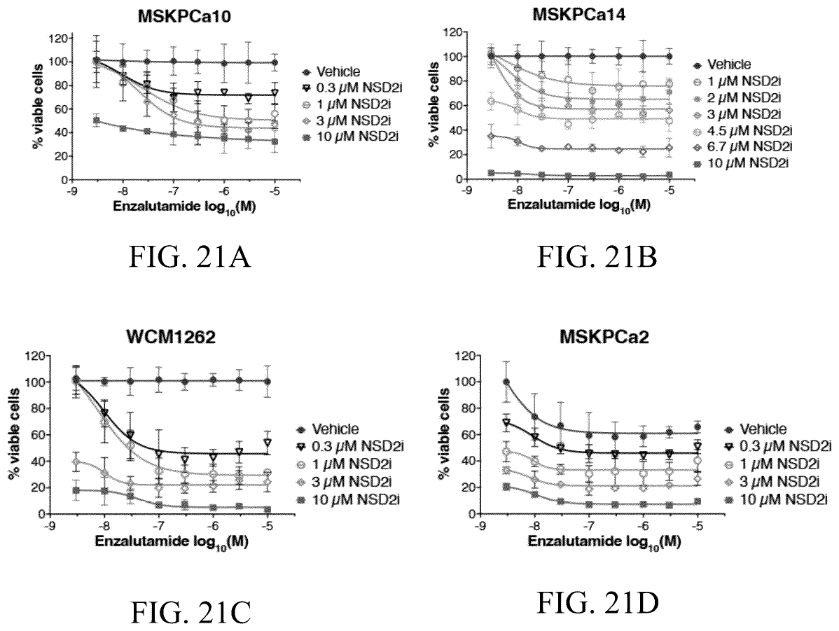

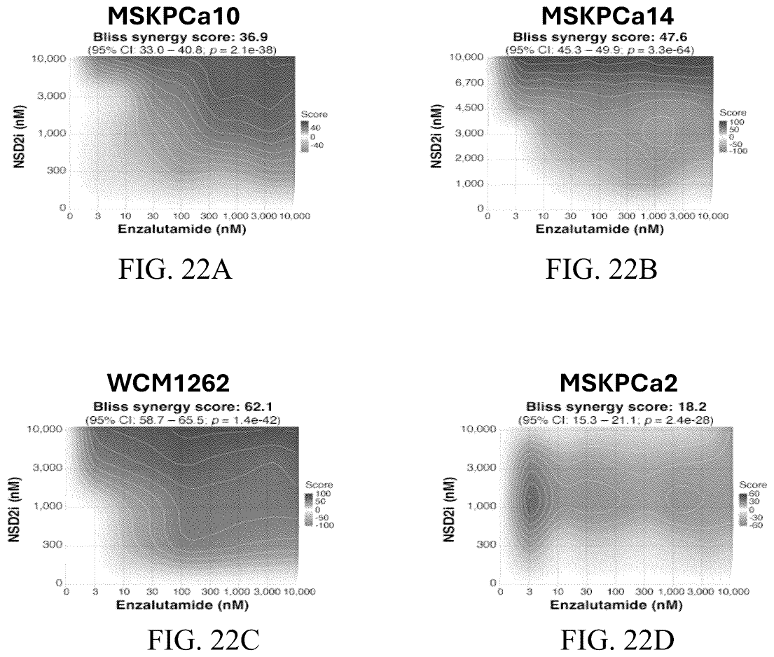

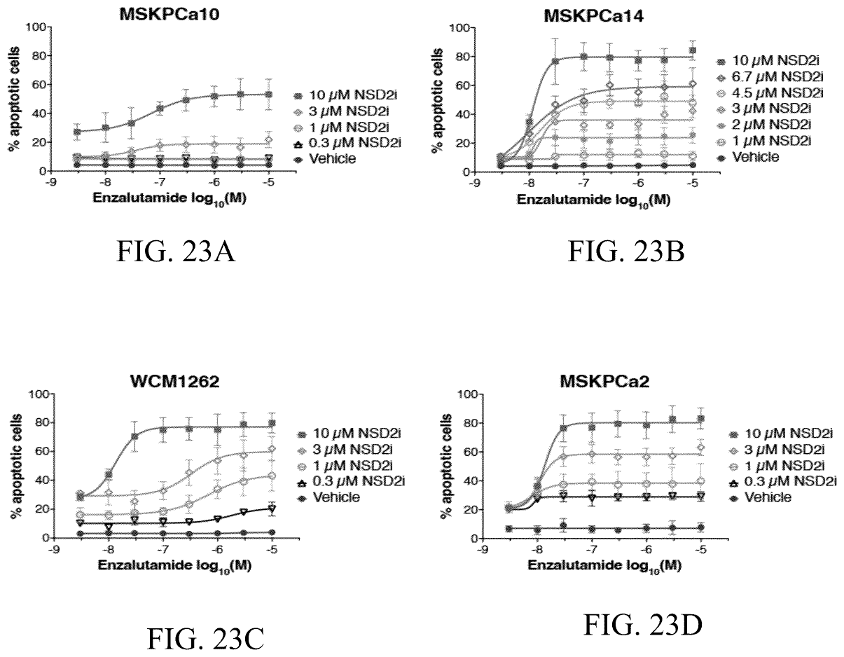

We then investigated the effects of combined treatment with NSD2iA and enzalutamide, using pre-treatment with NSD2iA for 3 weeks at concentrations ranging from 0.3 μM to 10 μM (FIG. 15). Increasing NSD2iA concentrations led to progressively decreased cell viability following enzalutamide treatment in each of the 5 human CRPC organoid lines (FIGS. 21A-D, 31A-B, 33A-E). We assessed the synergy between NSD2iA and enzalutamide by SynergyFinder analysis, which generated a high Bliss Synergy Score for each organoid line, supporting a synergistic effect (FIGS. 22A-D, 30A-B). In contrast, a similar treatment regimen using the EZH2 inhibitor PF-0681497 and enzalutamide on MSKPCa10 organoids did not suppress growth at any concentrations tested, generating a negative Bliss Synergy Score indicating lack of synergy or even antagonism (FIGS. 31A-B). Notably, combined treatment with NSD2iA and enzalutamide led to concentration-dependent induction of apoptosis, as detected by assays for caspase 3/7 activity (FIGS. 23A-D, 32, 33A-E).

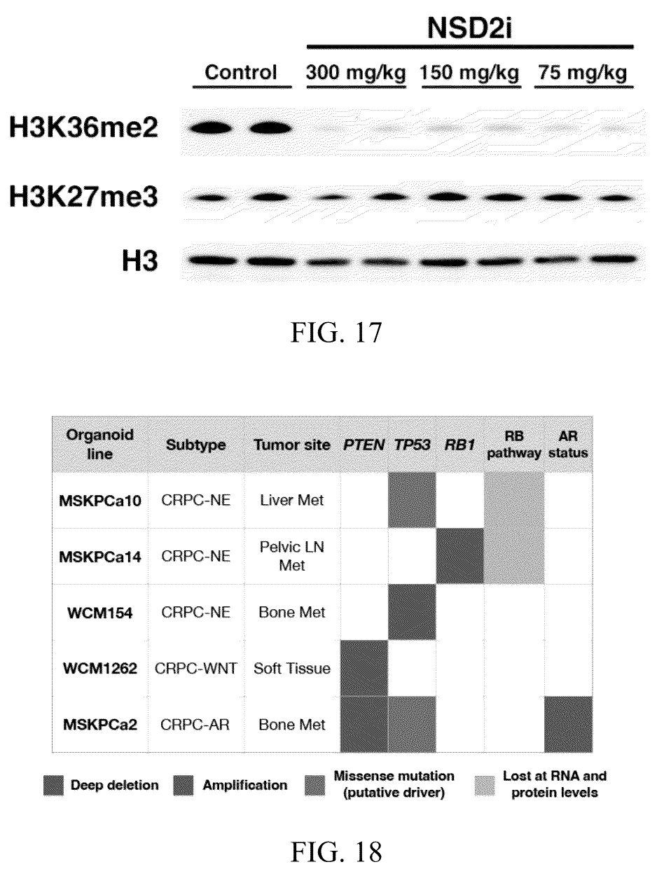

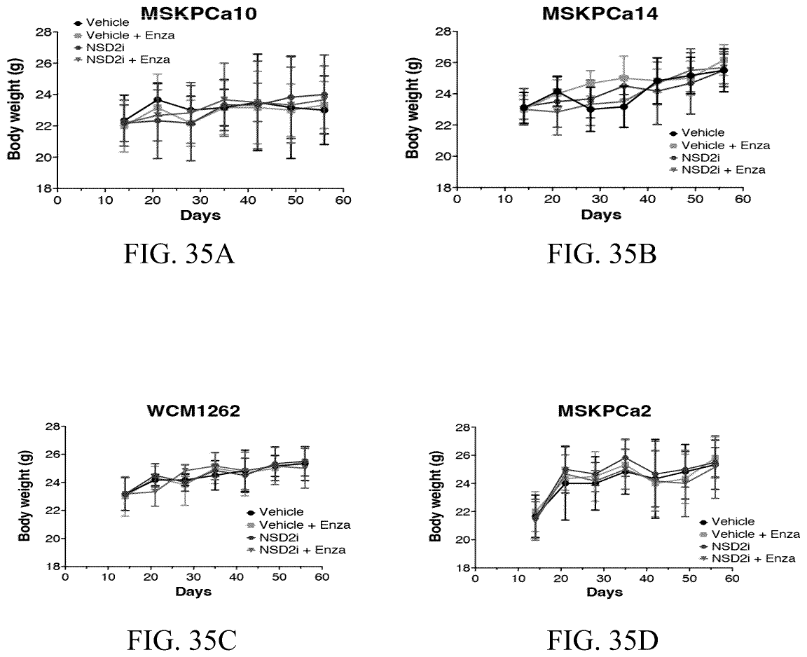

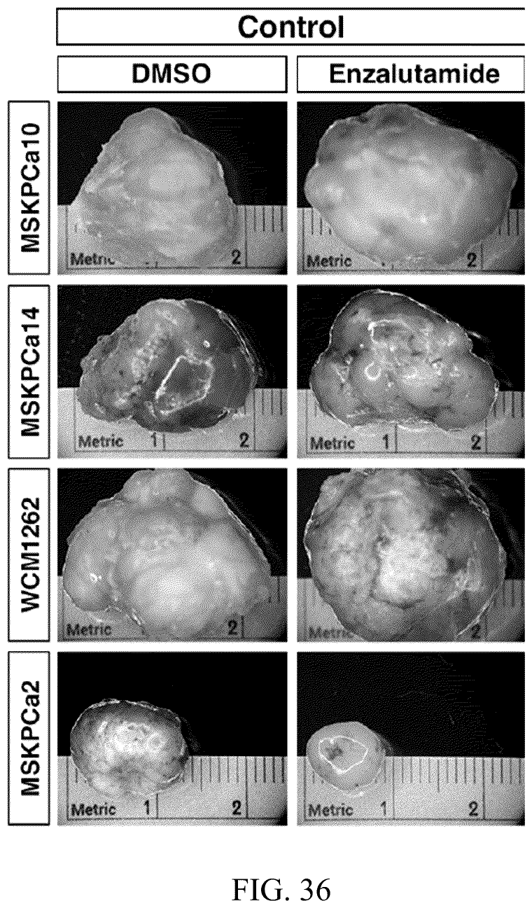

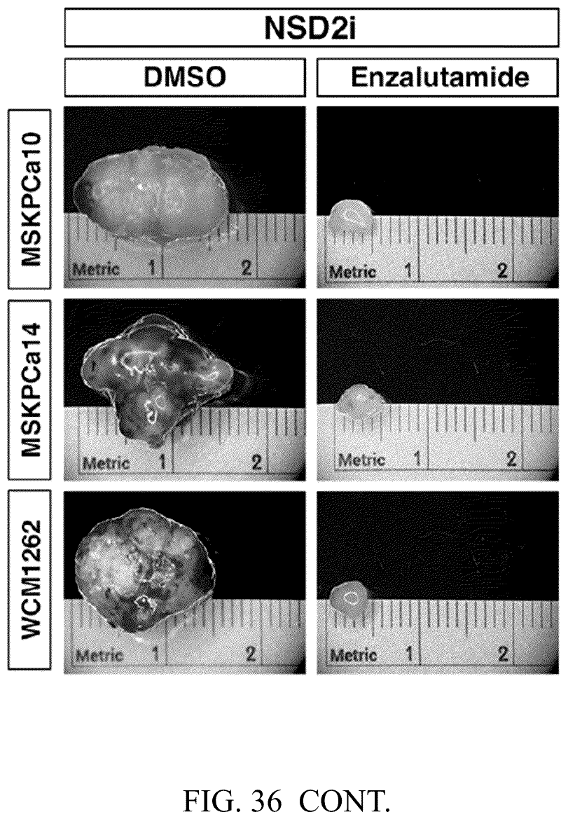

Finally, we assessed the effects of NSD2iA treatment in vivo, using subcutaneous grafts of human CRPC organoids in immunodeficient NOD/SCID mice. After treatment of WCM1262 grafts with NSD2iA administered daily for 5 days at 3 different concentrations, H3K36me2 levels were greatly reduced at each concentration tested (FIGS. 15, 17), without any adverse effects on mouse body weights (FIGS. 35A-D).

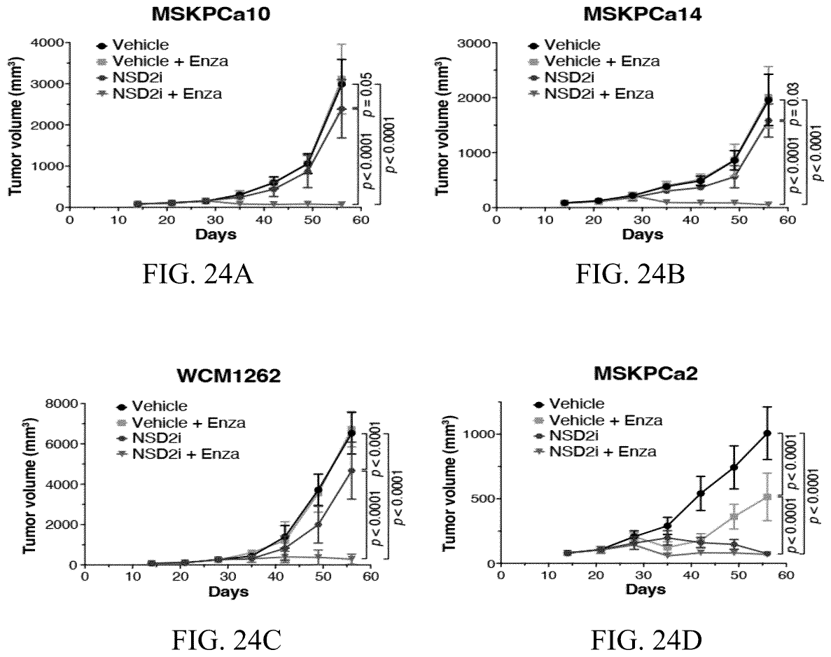

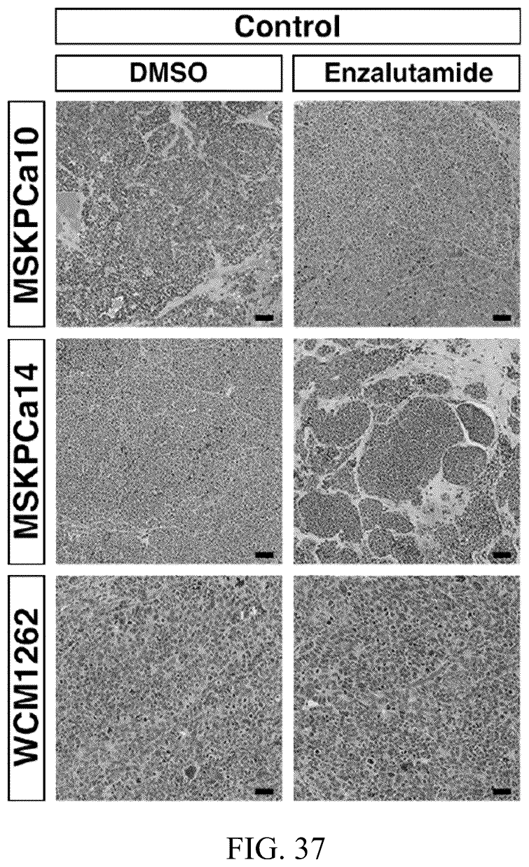

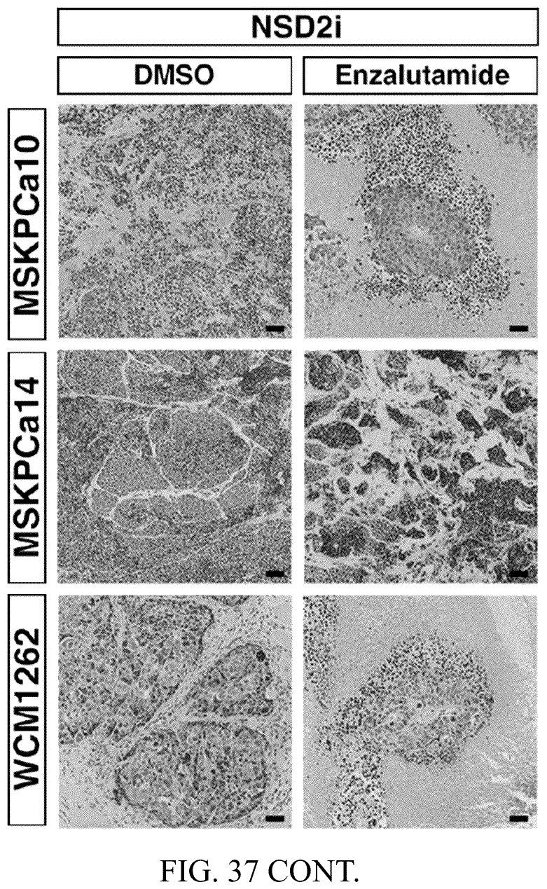

We then examined potential synergy with enzalutamide in vivo by daily pre-treatment of xenografts for 2 weeks with NSD2iA or DMSO followed by daily treatment with DMSO, enzalutamide alone, NSD2iA alone, or combined NSD2iA and enzalutamide for a further four weeks (FIGS. 15, 24A-D). Treatment with NSD2iA alone had modest effects on tumor growth for MSKPCa10, MSKPCa14, and WCM1262 xenografts, whereas combined treatment with enzalutamide resulted in strong tumor growth suppression in each case (FIGS. 24A-D, 36). Notably, the MSKPCa2 xenografts displayed a modest response to enzalutamide alone, but a strong response to NSD2iA alone or combined NSD2iA and enzalutamide (FIGS. 24A-D). Analyses of the histology of MSKPCa10, MSKPCa14, and WCM1262 xenografts treated with both NSD2iA and enzalutamide showed a loss of neuroendocrine phenotypes accompanied by overt necrosis and fibrosis as well as increased adenocarcinoma features in MSKPCa10 and WCM1262 (FIG. 37).

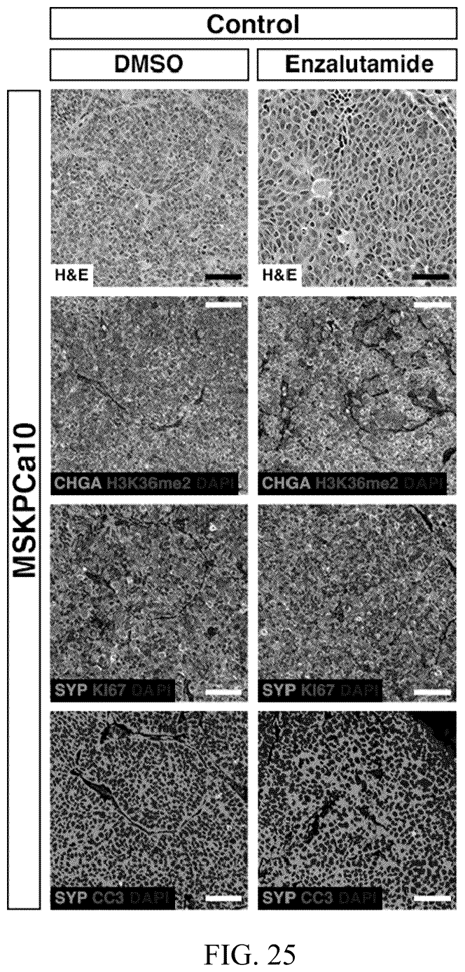

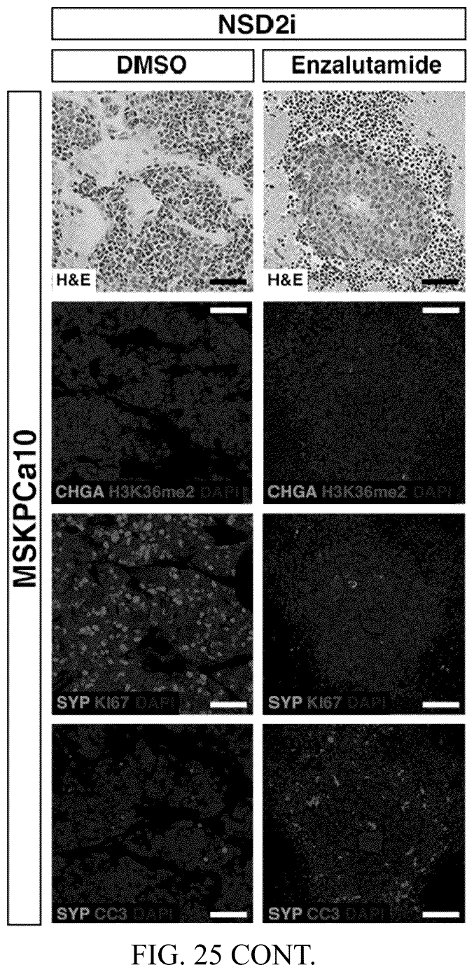

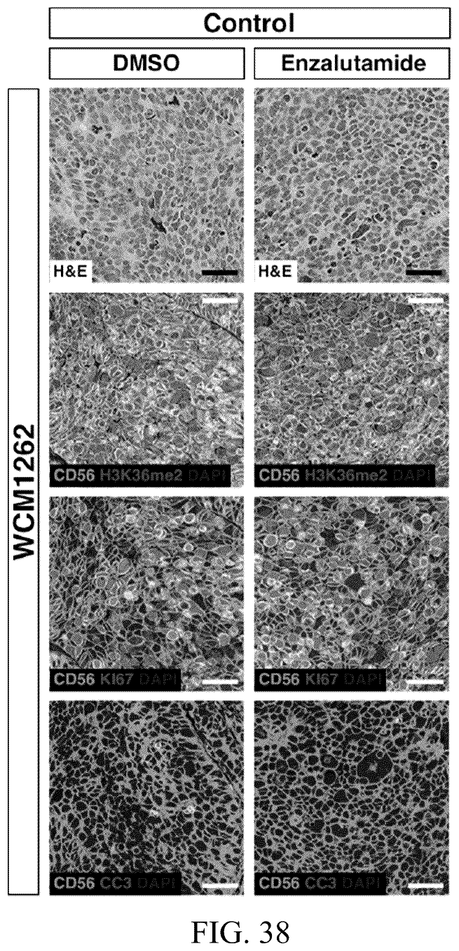

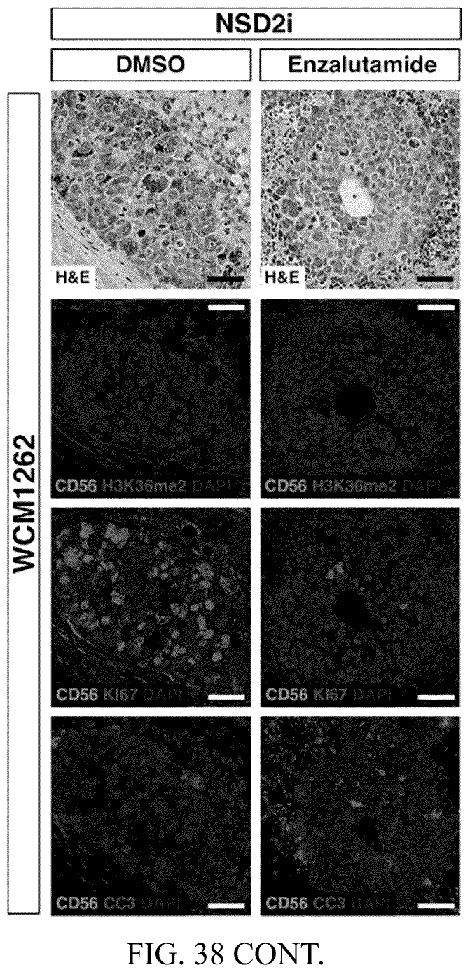

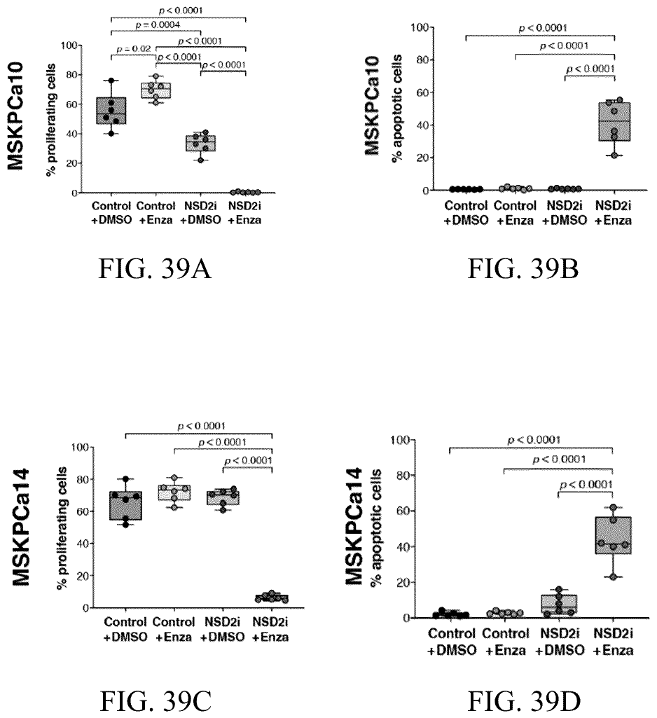

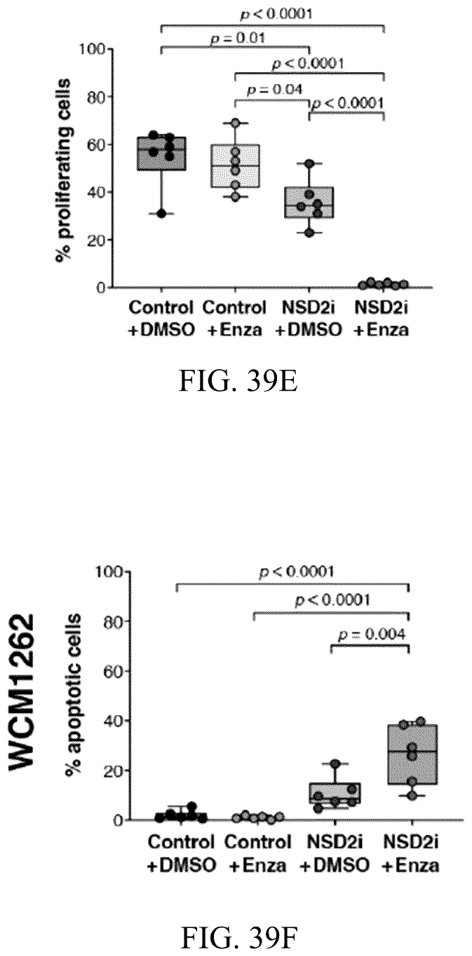

Consistent with these findings, the co-treated xenografts showed loss of H3K36me2 together with loss of the neuroendocrine markers CHGA and SYP in MSKPCa10 and MSKPCa14 as well as loss of CD56 in WCM1262 (FIGS. 25, 26, 37, 38). Interestingly, all of the co-treated xenografts displayed strong down-regulation of Ki67, indicating decreased cell proliferation, as well as up-regulation of cleaved caspase 3 (CC3), indicating apoptosis (FIGS. 25, 26, 38, 39A-F). Taken together, these findings indicate that combined inhibition of NSD2 and AR displays strong anti-tumor activity in vivo by reversing lineage plasticity, suppressing growth, and promoting tumor cell apoptosis in xenografts of human prostate organoids representing a range of CRPC subtypes.

### Methods

### Synthesis and Analysis of NSD2 Inhibitor

To assess the consequences of pharmacological targeting of NSD2, we used NSD2iA (FIG. 40), a small molecule that is similar to the active component of KTX-1001. We synthesized this parent compound using the approach described in Deng et al. (2023), with minor modifications (FIG. 41). The purified NSD2iA compound was then tested for its ability to inhibit the activity of a range of histone methyltransferases in vitro.

### Determination of IC50 for NSD2iA

The MTase-Glo Methyltransferase Assay kit (Promega) was used to measure the IC50 for NSD2iA with different KMTs. A 10 μl reaction system was assembled with 25 nM KMT enzyme (or enzyme complex), 0.5 μM SAM, 100 nM mononucleosome (EpiCypher), MTase-Glo Reagent (1×) and a series of diluted NSD2iA in reaction buffer (containing 50 mM Tris pH 8.0, 20 mM KCl, 5 mM MgCl2 and 10% glycerol) in a white 384-well microplate (Corning). NSD2iA was serially diluted from 30 μM to 0 in three-fold concentrations. Each reaction was performed in triplicate and incubated for 3 h at 30° C. Subsequently, 10 μl of MTase-Glo Detection solution was added and incubated for 1 h at room temperature. The reactions were detected by luminescence, relative activity of enzyme calculated with the initial data, and IC50 values analyzed using Prism v8.0.

### NSD2 Inhibitor Treatment of Organoids

We pre-treated mouse and human organoids with NSD2iA for 12 or 21 days, respectively, prior to 5 days co-treatment with NSD2iA and enzalutamide. Organoids were dissociated into single cells using TrypLE, and seeded at a density of 1,000 cells/well (mouse) in NE organoid culture medium without DHT in a 96-well low-attachment dish with 5% Matrigel (mouse) or as a total of 25,000 cells (human) in a 50 μl 80% Matrigel button in human NE culture medium without DHT. DMSO or NSD2iA at concentrations ranging from 0.3 μM to 10 μM were added on the day of plating and replenished every other day. Following pre-treatment, organoids were dissociated into single cells and seeded at a density of 5,000 cells/well (mouse) or 10,000 cells/well (human) in a 96-well low-attachment dish in the same culture medium. The next day, DMSO or the same concentration of NSD2iA were dispensed together with enzalutamide at 3-fold dilutions ranging from 3 nM to 10,000 nM in 0.1% DMSO. Cell viability was assayed after five days using CellTiter-Glo 3D (Promega G9683), with luminescence measured by a GloMax® Explorer multimode plate reader (Promega).

To detect apoptotic cell death, organoids were dissociated into single cells and seeded at a density of 2.5×10⁴ cells/well (human) in a 96-well low-attachment dish in the same culture medium. DMSO (negative control), 10 μM Bortezomib (Cayman Chemical 10008822) as a positive control, or NSD2iA were dispensed together with a 3-fold dilution series of enzalutamide from 3 nM to 10,000 nM in 0.1% DMSO. Apoptotic cell death was assayed after 24-48 h treatment using Caspase-Glo® 3/7 3D Assay (Promega G8981), with luminescence measured by a GloMax® Explorer multimode plate reader (Promega). Luminescence values were first normalized to the DMSO treated controls and then compared to 10 μM Bortezomib treatment, which induces 100% apoptotic cell death after 24 or 48 h. The resulting signals normalized to a percentage of maximal response were plotted using GraphPad Prism 9 software.

### Multi-Drug Synergy Analysis

To analyze synergy between NSD2iA and enzalutamide, the multi-dose combination response data generated from CellTiter-Glo 3D assays were input into SynergyFinder (https://synergyfinder.fimm.fi), a web application for interactive analysis and visualization of multi-drug combination response data. NSD2iA and enzalutamide drug combination responses were calculated based on the Bliss reference model using SynergyFinder (Ianevski et al., 2022). Deviations between observed and expected responses with positive and negative values denote synergy and antagonism respectively. For estimation of outlier measurements, the cNMF algorithm (Ianevski et al., 2019) implemented in SynergyFinder was utilized.

### Drug Treatment Assays in Vivo

For drug treatment, NSD2iA, enzalutamide, or DMSO were suspended in 1% carboxymethylcellulose and 0.1% Tween 80 in distilled water. To generate human prostate organoid grafts, 3×10⁶ dissociated cells in 100 μl 60% Matrigel and 40% human hepatocyte culture medium were injected subcutaneously into the flank of castrated NOD/SCID male mice. To access NSD2iA potency and specificity in vivo, 12 mice grafted with WCM1262 organoid cells with tumor volume of ~100 mm³ at three weeks after grafting received 75 mg/kg, 150 mg/kg, or 300 mg/kg NSD2iA or 0.5% DMSO by daily gavage via 20G needle for 5 days. Tumors were then harvested and dissociated into single cells for Western blot analysis of histone marks.

To investigate response to NSD2iA and enzalutamide, we used cohorts of 24 mice for each organoid line examined. In each cohort, mice with tumor volume of ~80-86 mm³ at two weeks after grafting received either 150 mg/kg NSD2iA or 0.5% DMSO by daily gavage via 20G needle for 14 days. The 12 mice in the NSD2iA treatment group were then subdivided into two groups for treatment with 150 mg/kg NSD2iA alone or 150 mg/kg NSD2iA with 10 mg/kg enzalutamide for another 4 weeks. The 12 mice from the DMSO control group were also subdivided for further treatment with DMSO or with 10 mg/kg enzalutamide for another 4 weeks. The tumors were harvested and imaged under a stereo microscope (Olympus SZX16 with DP71 digital camera) using Olympus DP Controller 3.3.1.292 (Olympus). Tumor tissues were fixed in 10% formalin for 24-48 h and processed in the Columbia Molecular Pathology Core Facility. Tumor volumes and growth curves were calculated as described above. Two-way ANOVA and Tukey's multiple comparison post-hoc testing were used to compare means among groups.

### Detection of Proliferation and Apoptosis in Tumor Sections

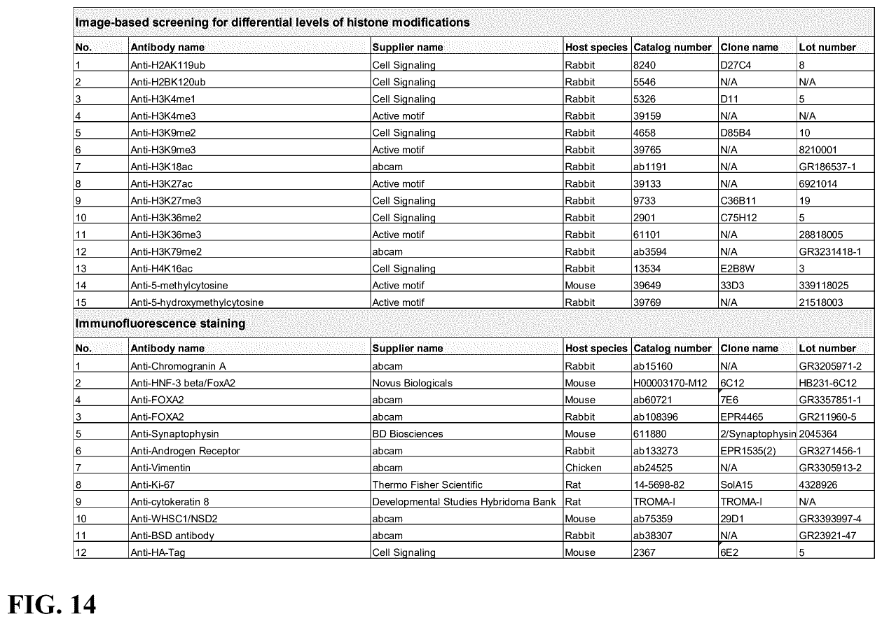

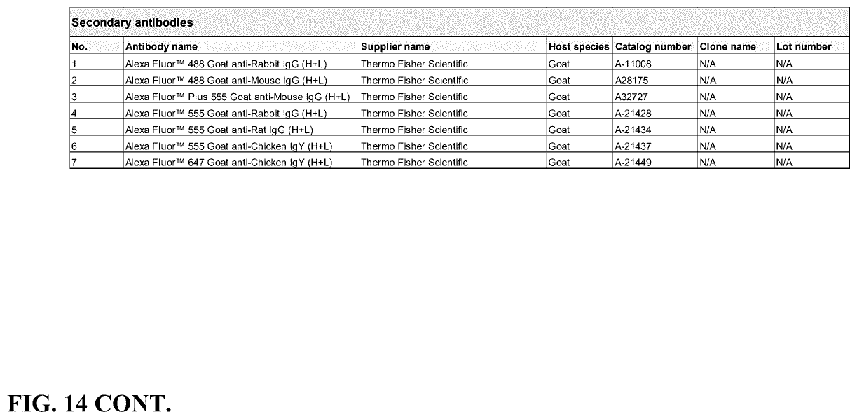

Tumor sections were stained for Ki67 or Cleaved Caspase-3 together with at least one lineage marker: chromogranin A, synaptophysin, or CD56. Antibodies used are listed in FIG. 14. Tiled scans of whole tumor tissue sections were imaged using a Leica STELLARIS 5 Confocal Microscope (Leica Microsystems). For quantitation of proliferation and apoptosis, six regions of interest were chosen randomly on the whole slide image using QuPath (Version 0.5.1) (Bankhead et al., 2017). A machine learning classifier was trained for identification of Ki67-positive or Cleaved Caspase-3-positive cells using the built-in functions in QuPath, and the percentage of Ki67- or Cleaved Caspase-3-positive cells were measured in each region of interest. An average percentage out of 6 regions was determined per xenograft specimen and plotted using Prism 9.

### REFERENCES FOR EXAMPLE 3

1. Gao, D., Vela, I., Sboner, A., Iaquinta, P. J., Karthaus, W. R., Gopalan, A., Dowling, C., Wanjala, J. N., Undvall, E. A., Arora, V. K., Wongvipat, J., Kossai, M., Ramazanoglu, S., Barboza, L. P., Di, W., Cao, Z., Zhang, Q. F., Sirota, I., Ran, L., MacDonald, T. Y., Beltran, H., Mosquera, J. M., Touijer, K. A., Scardino, P. T., Laudone, V. P., Curtis, K. R., Rathkopf, D. E., Morris, M. J., Danila, D. C., Slovin, S. F., Solomon, S. B., Eastham, J. A., Chi, P., Carver, B., Rubin, M. A., Scher, H. I., Clevers, H., Sawyers, C. L. and Chen, Y. (2014). Organoid cultures derived from patients with advanced prostate cancer. Cell 159:176-187. PMID: 25201530. PMCID: 4237931.
2. Tang, F., Xu, D., Wang, S., Wong, C. K., Martinez-Fundichely, A., Lee, C. J., Cohen, S., Park, J., Hill, C. E., Eng, K., Bareja, R., Han, T., Liu, E. M., Palladino, A., Di, W., Gao, D., Abida, W., Beg, S., Puca, L., Meneses, M., de Stanchina, E., Berger, M. F., Gopalan, A., Dow, L. E., Mosquera, J. M., Beltran, H., Sternberg, C. N., Chi, P., Scher, H. I., Sboner, A., Chen, Y. and Khurana, E. (2022). Chromatin profiles classify castration-resistant prostate cancers suggesting therapeutic targets. Science 376: eabe1505. PMID: 35617398. PMCID: PMC9299269.
3. Dilworth, D., Hanley, R. P., Ferreira de Freitas, R., Allali-Hassani, A., Zhou, M., Mehta, N., Marunde, M. R., Ackloo, S., Carvalho Machado, R. A., Khalili Yazdi, A., Owens, D. D. G., Vu, V., Nie, D. Y., Alqazzaz, M., Marcon, E., Li, F., Chau, I., Bolotokova, A., Qin, S., Lei, M., Liu, Y., Szewczyk, M. M., Dong, A., Kazemzadeh, S., Abramyan, T., Popova, I. K., Hall, N. W., Meiners, M. J., Cheek, M. A., Gibson, E., Kireev, D., Greenblatt, J. F., Keogh, M. C., Min, J., Brown, P. J., Vedadi, M., Arrowsmith, C. H., Barsyte-Lovejoy, D., James, L. I. and Schapira, M. (2022). A chemical probe targeting the PWWP domain alters NSD2 nucleolar localization. Nat Chem Biol 18:56-63. PMID: 34782742. PMCID: PMC9189931.
4. Puca, L., Bareja, R., Prandi, D., Shaw, R., Benelli, M., Karthaus, W. R., Hess, J., Sigouros, M., Donoghue, A., Kossai, M., Gao, D., Cyrta, J., Sailer, V., Vosoughi, A., Pauli, C., Churakova, Y., Cheung, C., Deonarine, L. D., McNary, T. J., Rosati, R., Tagawa, S. T., Nanus, D. M., Mosquera, J. M., Sawyers, C. L., Chen, Y., Inghirami, G., Rao, R. A., Grandori, C., Elemento, O., Sboner, A., Demichelis, F., Rubin, M. A. & Beltran, H. Patient derived organoids to model rare prostate cancer phenotypes. Nat Commun 9, 2404 (2018).
5. Ianevski, A., Giri, A. K. & Aittokallio, T. SynergyFinder 3.0: an interactive analysis and consensus interpretation of multi-drug synergies across multiple samples. Nucleic Acids Res 50, W739-W743 (2022).
6. Ianevski, A., Giri, A. K., Gautam, P., Kononov, A., Potdar, S., Saarela, J., Wennerberg, K. & Aittokallio, T. Prediction of drug combination effects with a minimal set of experiments. Nat Mach Intell 1, 568-577 (2019).
7. Bankhead, P., Loughrey, M. B., Fernandez, J. A., Dombrowski, Y., McArt, D. G., Dunne, P. D., McQuaid, S., Gray, R. T., Murray, L. J., Coleman, H. G., James, J. A., Salto-Tellez, M. & Hamilton, P. W. QuPath: Open source software for digital pathology image analysis. Sci Rep 7, 16878 (2017).
8. Deng, H., Liu, J., Oyang, C., Xiao, Q., Xun, G. & Zeng, H. Piperidyl-methyl-purine amines as NSD2 inhibitors and anti-cancer agents. US patent 2023/0002388 A1 (2023).

1. A method of treating or preventing prostate cancer in a subject in need thereof, the method comprising administering to the subject a pharmaceutical composition comprising a therapeutically effective amount of a Nuclear Receptor Binding Set Domain Protein 2 (NSD2) inhibitor.

2. The method of claim 1, wherein the prostate cancer is an advanced stage or an end-stage cancer.

3. The method of claim 1, wherein the prostate cancer is castration-resistant prostate cancer (CRPC), neuroendocrine prostate cancer (NEPC), or neuroendocrine castration resistant prostate cancer (CRPC-NE).

4. The method of claim 1, wherein the prostate cancer is CRPC with androgen receptor expression (CRPC-AR) or CRPC with an activation of the Wnt signaling pathway (CRPC-WNT).

5. The method of claim 3, wherein the CRPC has decreased or no androgen receptor expression or sensitivity to one or more androgen receptor inhibitors.

6. The method of claim 1, wherein the NSD2 inhibitor comprises 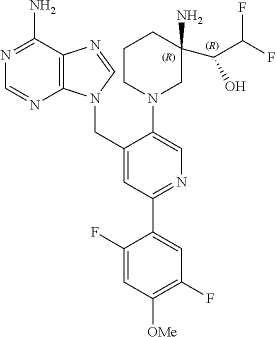
or a pharmaceutically acceptable salt thereof.

7. The method of claim 1, wherein the NSD2 inhibitor comprises UNC6934, 3-hydrazinoquinoxaline-2-thiol (MCTP-39), or MS159.

8. The method of claim 1, wherein the NSD2 inhibitor is administered in combination with another NSD2 inhibitor or one or more NSD2 activity modulator.

9. The method of claim 1, wherein the pharmaceutical composition comprises a small interfering RNA specific for a messenger RNA sequence encoding the NSD2 protein.

10. The method of claim 1, wherein the NSD2 inhibitor comprises a CRISPR cassette specific for the nucleotide gene sequence encoding a NSD2 gene or controlling transcription of a NSD2 gene.

11. The method of claim 1, wherein the pharmaceutical composition is administered before, after, or in combination with an anti-androgen treatment.

12. The method of claim 11, wherein the anti-androgen treatment comprises administration of enzalutamide, abiraterone, apalutamide, bicalutamide, darolutamide, flutamide, or nilutamide.

13. The method of claim 12, wherein the anti-androgen treatment comprises administration of enzalutamide.

14. The method of claim 11, wherein the anti-androgen treatment comprises administration of one or more luteinizing hormone-releasing hormone (LHRH) agonists.

15. The method of claim 11, wherein the method decreases tumor size, decreases tumor number, or prevents metastasis.

16. The method of claim 1, wherein administration of the NSD2 inhibitor to the subject in combination with an anti-androgen treatment prevents cancer metastasis.

17. The method of claim 1, wherein the composition is administered before, after, or in combination with a radiation therapy.

18. The method of claim 1, wherein the subject is a human.

19. A method of inducing prostate cancer sensitivity to one or more androgen receptor (AR) inhibitors in a subject in need thereof, the method comprising administering to the subject a pharmaceutical composition comprising a therapeutically effective amount of a Nuclear Receptor Binding Set Domain Protein 2 (NSD2) inhibitor.

20. The method of claim 19, wherein the prostate cancer is an advanced stage or an end-stage cancer.

21. The method of claim 19, wherein the prostate cancer is castration-resistant prostate cancer (CRPC), neuroendocrine prostate cancer (NEPC), or neuroendocrine castration-resistant prostate cancer (CRPC-NE).

22. The method of claim 19, wherein the prostate cancer is CRPC with androgen receptor expression (CRPC-AR) or CRPC with an activation of the Wnt signaling pathway (CRPC-WNT).

23. The method of claim 21, wherein the CRPC has decreased or no androgen receptor expression or sensitivity to one or more androgen receptor inhibitors.

24. The method of claim 19, wherein the NSD2 inhibitor comprises 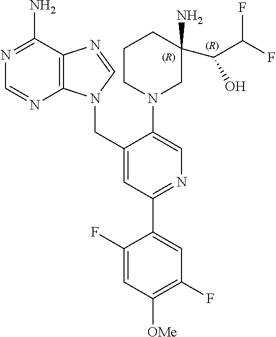
or a pharmaceutically acceptable salt thereof.

25. The method of claim 19, wherein the NSD2 inhibitor comprises UNC6934, 3-hydrazinoquinoxaline-2-thiol (MCTP-39), or MS159.

26. The method of claim 19, wherein the NSD2 inhibitor is administered in combination with another NSD2 inhibitor or one or more NSD2 activity modulator.

27. The method of claim 19, wherein the AR inhibitor comprises enzalutamide, abiraterone, apalutamide, bicalutamide, darolutamide, flutamide, or nilutamide.

28. The method of claim 19, wherein the composition is administered before, after, or in combination with a radiation therapy.

29. The method of claim 19, wherein the subject is a human.
